(19) United States
(12) Patent Application Publication
(10) Pub. No.: US 2024/0307462 A1
(43) Pub. Date: Sep. 19, 2024

(54) COMPOSITION FOR INDUCING PROLIFERATION OR ACCUMULATION OF REGULATORY T CELLS

(71) Applicant: The University of Tokyo, Tokyo (JP)

(72) Inventors: Kenya Honda, Tokyo (JP); Koji Atarashi, Tokyo (JP); Kikuji Itoh, Tokyo (JP); Takeshi Tanoue, Tokyo (JP)

(73) Assignee: The University of Tokyo, Tokyo (JP)

(21) Appl. No.: 18/676,470

(22) Filed: May 28, 2024

Related U.S. Application Data
(63) Continuation of application No. 17/371,692, filed on Jul. 9, 2021.
Application No. 17/371,692 is a continuation of application No. 16/780,116, filed on Feb. 3, 2020, now Pat. No. 11,090,343.
Application No. 16/780,116 is a continuation of application No. 16/425,030, filed on May 29, 2019, now Pat. No. 10,555,978.
Application No. 16/425,030 is a continuation of application No. 16/389,380, filed on Apr. 19, 2019, now Pat. No. 10,588,925.
Application No. 16/389,380 is a continuation of application No. 16/171,558, filed on Oct. 26, 2018, now Pat. No. 10,328,108.
Application No. 16/171,558 is a continuation of application No. 16/117,054, filed on Aug. 30, 2018, now Pat. No. 10,322,150.
Application No. 16/117,054 is a continuation of application No. 15/730,203, filed on Oct. 11, 2017, now Pat. No. 10,092,603.
Application No. 15/730,203 is a continuation of application No. 15/216,015, filed on Jul. 21, 2016, now Pat. No. 9,801,933.
Application No. 15/216,015 is a continuation of application No. 14/492,850, filed on Sep. 22, 2014, now Pat. No. 9,433,652.
Application No. 14/492,850 is a continuation of application No. 13/701,467, filed on Feb. 11, 2013, now abandoned.

(30) Foreign Application Priority Data
Jun. 4, 2010 (JP) 2010-129134
Dec. 3, 2010 (WO) PCT/JP2010/071746

(51) Int. Cl. A61K 35/742; A01K 67/0275; A61K 9/00; A61K 9/48; A61K 35/00; A61K 35/74; A61K 39/00; A61K 39/08; A61K 39/39; A61K 45/00; A61K 45/06; C12Q 1/689; G01N 33/50

(52) U.S. Cl. CPC A61K 35/742; A01K 67/0275; A61K 9/0053; A61K 9/48; A61K 35/74; A61K 39/0008; A61K 39/08; A61K 39/39; A61K 45/00; A61K 45/06; C12Q 1/689; G01N 33/505; A01K 2267/0325; A61K 35/00; A61K 2039/52; A61K 2039/542; A61K 2039/55594; A61K 2039/57; C12Q 2600/158; G01N 2333/33; G01N 2500/10

(57) ABSTRACT

It was found that bacteria belonging to the genus Clostridium induce accumulation of regulatory T cells (Treg cells) in the colon. Moreover, the present inventors found that regulatory T cells (Treg cells) induced by from these bacteria suppressed proliferation of effector T-cells. From these findings, the present inventors found that the use of bacteria belonging to the genus Clostridium or a physiologically active substance derived therefrom made it possible to induce proliferation or accumulation of regulatory T cells (Treg cells), and further to suppress immune functions.

### RELATED APPLICATIONS

This application is a continuation of U.S. application Ser. No. 17/371,692, filed Jul. 9, 2021, which is a continuation of U.S. Application Ser. No. 16/780,116, filed Feb. 3, 2020, now issued as U.S. Pat. No. 11,090,343, which is a continuation of U.S. application Ser. No. 16/425,030, filed May 29, 2019, now issued as U.S. Pat. No. 10,555,978, which is a continuation of U.S. application Ser. No. 16/389,380, filed Apr. 19, 2019, now issued as U.S. Pat. No. 10,588,925, which is a continuation of U.S. application Ser. No. 16/171,558, filed Oct. 26, 2018, now issued as U.S. Pat. No. 10,328,108, which is a continuation of U.S. application Ser. No. 16/117,054, filed Aug. 30, 2018, now issued as U.S. Pat. No. 10,322,150, which is a continuation of U.S. application Ser. No. 15/730,203, filed Oct. 11, 2017, now issued as U.S. Pat. No. 10,092,603, which is a continuation of U.S. application Ser. No. 15/216,015, filed Jul. 21, 2016, now issued as U.S. Pat. No. 9,801,933, which is a continuation of U.S. application Ser. No. 14/492,850, filed Sep. 22, 2014, now issued as U.S. Pat. No. 9,433,652, which is a continuation of U.S. application Ser. No. 13/701,467, filed Feb. 11, 2013, which is a national stage filing under 35 U.S.C. 371 of International Application PCT/JP2011/063302, filed Jun. 3, 2011, which claims the benefit of and priority to JP 2010-129134, filed Jun. 4, 2010 and PCT/JP2010/071746, filed Dec. 3, 2010. The entire teachings of the referenced applications are incorporated by reference herein.

### REFERENCE TO AN ELECTRONIC SEQUENCE LISTING

The contents of the electronic sequence listing (P074570002US18-SEQ-NTJ.xml; Size: 150,557 bytes; and Date of Creation: May 28, 2024) are herein incorporated by reference in their entirety.

### TECHNICAL FIELD

The present invention relates to a composition which has an effect of inducing proliferation or accumulation of regulatory T cells, and which comprises, as an active ingredient, bacteria belonging to the genus Clostridium, a physiologically active substance derived from the bacteria, bacterial spores, or the like. The present invention also relates to a method for inducing proliferation or accumulation of regulatory T cells, as well as a method for inhibiting such proliferation or accumulation. Moreover, the present invention relates to a vaccine composition containing at least one strain of bacteria belonging to the genus Clostridium or a spore of bacteria, as well as a method for treating or preventing at least one disease or condition selected from infectious diseases and autoimmune diseases by administering the vaccine composition to an individual in need thereof. The present invention also relates to a method for screening for a compound that promotes proliferation or accumulation of regulatory T cells, as well as a non-human mammal which is used in this method, and in which a reporter gene is expressed under control of IL-10 gene expression.

### BACKGROUND ART

Hundreds of species of commensal microorganisms are harbored in gastrointestinal tracts of mammals, and intimately interact with the host immune systems. Results of researches using germ-free (GF) animals have shown that the commensal microorganisms exert great influences on the development of mucosal immune systems such as histogenesis of Peyer's patches (PPs) and isolated lymphoid follicles (ILFs), secretion of antimicrobial peptides from epithelium, and accumulation of unique lymphocytes in mucosal tissues, the unique lymphocytes including immunoglobulin A-producing plasma cells, intraepithelial lymphocytes, IL-17-producing CD4-positive T cells (Th 17), and IL-22-producing NK-like cells (Non-Patent Documents 1 to 7). Consequently, the presence of intestinal bacteria enhances protective functions of the mucous membranes, providing the hosts with robust immune responses against pathogenic microbes invading the bodies. On the other hand, the mucosal immune systems maintain unresponsiveness to dietary antigens and harmless microbes (Non-Patent Document 3). For this reason, abnormality in the regulation of cross-talk between commensal bacteria and an immune system (intestinal dysbiosis) may lead to overly robust immune response to environmental antigens, so that inflammatory bowel disease (IBD) is caused (Non-Patent Documents 8 to 10).

Results of recent studies have shown that individual commensal bacteria control differentiation of their specific immune cells in the mucosal immune system. For example, Bacteroides fragilis, which is a commensal bacterium in humans, specifically induces a systemic Th1 cell response and a mucosal IL-10-producing T cell response in mice, and plays a role in protecting the host from colitis, which would otherwise be caused by a pathogen (Non-Patent Document 3). Segmented filamentous bacteria, which are intestinal commensal bacteria in mice, are shown to induce mucosal Th17 cell response and thereby to enhance resistance against infection of gastrointestinal tracts of the host with a pathogen (Non-Patent Documents 11 to 13). In addition, short-chain fatty acids derived from several commensal bacteria are known to suppress intestinal inflammation (Non-Patent Document 14). Moreover, it is presumed that the presence of some species of intestinal microbiota exerts a great influence on the differentiation of regulatory T cells (hereafter referred to as “Treg cells”) which maintain homeostasis of the immune system.

Meanwhile, regulatory T cells which have been identified as a subset suppressing immunity are CD4⁺ T cells in which a transcription factor Foxp3 is expressed, and are known to play an important role in maintaining immunological homeostasis (Non-Patent Documents 8, 9, 15, and 16). Moreover, it has been known that the Foxp3-expressing cells are present in a large number especially in the colon, and only Treg cells present locally in the colon constantly expresses IL-10, which is an immunosuppressive cytokine, at a high level (Non-Patent Document 17). It is also known that animals having CD4⁺ Foxp3⁺ cells from which IL-10 is specifically removed develop inflammatory bowel disease (Non-Patent Document 18).

Accordingly, if the mechanism of the induction of Treg cells which produce IL-10 in the colon at a high level is elucidated, immunosuppression can be enhanced, which in turn can be applied to treatment of autoimmune diseases such as inflammatory bowel disease, as well as to organ transplantation.

However, mechanisms of how a large number of Treg cells come to be present in the colon and how the Treg cells produce IL-10 in the colon at a high level are still unclear. Moreover, it is also still unclear what species of bacteria constituting the intestinal commensal bacterial flora exerts the influence on the induction of regulatory T cells.

### CITATION LIST

### Non Patent Literature

[NPL 1]J. J. Cebra, “Am J Clin Nutr”, May 1999, 69, 1046S
[NPL 2]A. J. Macpherson, N. L. Harris, “Nat Rev Immunol”, June 2004, 4, 478
[NPL 3]J. L. Round, S. K. Mazmanian, “Nat Rev Immunol”, May 2009, 9, 313
[NPL 4]D. Bouskra et al., “Nature”, Nov. 27, 2008, 456, 507
[NPL 5]K. Atarashi et al., “Nature”, Oct. 9, 2008, 455, 808
[NPL 6]Ivanov, II et al., “Cell Host Microbe”, Oct. 16, 2008, 4, 337
[NPL 7]S. L. Sanos et al., “Nat Immunol”, January 2009, 10, 83
[NPL 8]M. A. Curotto de Lafaille, J. J. Lafaille, “Immunity”, May 2009, 30, 626
[NPL 9]M. J. Barnes, F. Powrie, “Immunity”, Sep. 18, 2009, 31, 401
[NPL 10]W. S. Garrett et al., “Cell”, Oct. 5, 2007, 131, 33
[NPL 11]Ivanov, II et al., “Cell”, Oct. 30, 2009, 139, 485.
[NPL 12]V. Gaboriau-Routhiau et al., “Immunity”, Oct. 16, 2009, 31, 677
[NPL 13]N. H. Salzman et al., “Nat Immunol”, 11, 76.
[NPL 14]K. M. Maslowski et al., “Nature”, Oct. 29, 2009, 461, 1282
[NPL 15]L. F. Lu, A. Rudensky, “Genes Dev”, Jun. 1, 2009, 23, 1270
[NPL 16]S. Sakaguchi, T. Yamaguchi, T. Nomura, M. Ono, “Cell”, May 30, 2008, 133, 775
[NPL 17]C. L. Maynard et al., “Nat Immunol”, September 2007, 8, 931
[NPL 18]Y. P. Rubtsov et al., “Immunity”, April 2008, 28, 546

### SUMMARY OF INVENTION

### Technical Problem

The present invention has been made in view of the above-described problems of the conventional techniques. Accordingly, an object of the present invention is to identify intestinal commensal bacteria which induce the proliferation or accumulation of regulatory T cells. Another object of the present invention is to provide compositions or the like which comprise the identified intestinal commensal bacteria or a physiologically active substance derived therefrom, and which thus have induce the proliferation or accumulation of regulatory T cells (Treg cells).

### Solution to Problem

The present inventors have made earnest studies to solve the above-described problems. As a result, the present inventors have found that a chloroform-treated fraction and a spore-forming fraction of a fecal sample obtained from a mammal induces accumulation of regulatory T cells (Treg cells) in the colon. Moreover, the present inventors have found that bacteria belonging to the genus Clostridium induce proliferation or accumulation of regulatory T cells in the colon. The present inventors have also found that the regulatory T cells induced by these bacteria suppress proliferation of effector T cells. Furthermore, the present inventors have also found that colonization of bacteria belonging to the genus Clostridium and resultant proliferation or accumulation of Treg cells regulate local and systemic immune responses.

From these findings, the present inventors have found that the use of bacteria belonging to the genus Clostridium, spores thereof, or a physiologically active substance derived therefrom makes it possible to induce the proliferation or accumulation of regulatory T cells (Treg cells), and further to suppress immune functions.

More specifically, the present invention has the following aspects:
(1) A composition that induces proliferation or accumulation of regulatory T cells, the composition comprising, as an active ingredient, at least one substance selected from the group consisting of the following (a) to (c):
   (a) bacteria belonging to the genus Clostridium or a physiologically active substance derived from the bacteria;
   (b) a spore-forming fraction of a fecal sample obtained from a mammal or a culture supernatant of the fraction; and
   (c) a chloroform-treated fraction of a fecal sample obtained from a mammal or a culture supernatant of the fraction.
(2) Any of the compositions described herein wherein
   the regulatory T cells are transcription factor Foxp3-positive regulatory T cells or IL-10-producing regulatory T cells.
(3) The composition according to any one of (1) and (2), wherein
   the composition has an immunosuppressive effect.
(4) The composition according to any one of (1) to (3), wherein
   the composition is a pharmaceutical composition.
(5) A method for inducing proliferation or accumulation of regulatory T cells in an individual (e.g., an individual in need thereof, such as an individual in need of induction of proliferation or accumulation of regulatory T cells), the method comprising a step of administering, to the individual, at least one substance selected from the group consisting of the following (a) to (c):
   (a) bacteria belonging to the genus Clostridium or a physiologically active substance derived from the bacteria;
   (b) a spore-forming fraction of a fecal sample obtained from a mammal or a culture supernatant of the fraction; and
   (c) a chloroform-treated fraction of a fecal sample obtained from a mammal or a culture supernatant of the fraction.
(6) A method for inducing proliferation or accumulation of regulatory T cells in an individual (e.g., an individual in need thereof, such as an individual in need of induction of proliferation or accumulation of regulatory T cells), the method comprising a step of administering an antibiotic against Gram-negative bacteria to the individual. And the antibiotic can be administered alone or in combination with at least one substance selected from the group consisting of the following (a) to (c):
   (a) bacteria belonging to the genus Clostridium or a physiologically active substance derived from the bacteria;
   (b) a spore-forming fraction of a fecal sample obtained from a mammal or a culture supernatant of the fraction; and
   (c) a chloroform-treated fraction of a fecal sample obtained from a mammal or a culture supernatant of the fraction.
(7) A method for inducing proliferation or accumulation of regulatory T cells in an individual, the method comprising a step of administering, to the individual, at least one substance selected from the group consisting of almond skin, inulin, oligofructose, raffinose, lactulose, pectin, hemicellulose, amylopectin, acetyl-Co A, biotin, beet molasses, yeast extracts, and resistant starch.
(8) The method according to any one of (5) to (7), wherein
   a therapeutic composition is further administered to the individual.
   Note that, the “therapeutic composition” here is meant to be something other than (a)-(c) described in (5) and (6), the antibiotic against Gram-negative bacteria described in (6), or the substances described in (7).
(9) The method according to (8), wherein
   the therapeutic composition is at least one composition selected from the group consisting of corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, anti-cholinergic decongestants, mast-cell stabilizers, monoclonal anti-IgE antibodies, vaccines, and combinations thereof.
(10) The method according to any one of (5) to (9), wherein
   one measurement selected from the group consisting of promotion of IL-10 expression, promotion of CTLA4 expression, promotion of IDO expression, and suppression of IL-4 expression is used as an index of the induction of proliferation or accumulation of regulatory T cells in the individual.
(11) A method for inhibiting proliferation or accumulation of regulatory T cells in an individual (e.g., an individual thereof), the method comprising a step of administering an antibiotic against Gram-positive bacteria to the individual.
(12) The composition according to any one of (5) to (11), wherein
   the regulatory T cells are transcription factor Foxp3-positive regulatory T cells or IL-10-producing regulatory T cells.
(13) A vaccine composition comprising at least one substance selected from the group consisting of the following (a) to (c):
   (a) bacteria belonging to the genus Clostridium;
   (b) a spore of bacteria in a spore-forming fraction of a fecal sample obtained from a mammal; and
   (c) bacteria in a chloroform-treated fraction of a fecal sample obtained from a mammal.
(14) A method for treating aiding in treating, reducing the severity of, or preventing at least one disease selected from infectious diseases and autoimmune diseases in an individual (e.g., an individual in need thereof, such as an individual in need of treatment, reduction in the severity of or prevention of at least one such disease), the method comprising administering the vaccine composition according to (13) to the individual.
(15) A method for screening for a compound having an activity to promote proliferation or accumulation of regulatory T cells, the method comprising:
   (I) preparing a test substance from at least one substance selected from the group consisting of the following (a) to (c):
      (a) bacteria belonging to the genus Clostridium or a physiologically active substance derived from the bacteria;
      (b) a spore-forming fraction of a fecal sample obtained from a mammal or a culture supernatant of the fraction; and
      (c) a chloroform-treated fraction of a fecal sample obtained from a mammal or a culture supernatant of the fraction.
   (II) preparing non-human mammals in which a reporter gene is to be expressed under control of IL-10 gene expression;
   (III) bringing the test substance into contact with the non-human mammal;
   (IV) after the contact with the test substance, detecting cells expressing the reporter gene in a CD4⁺ Foxp3⁺ cell group of the non-human mammal, and determining the number of cells in the CD4⁺ Foxp3⁺ cell group expressing the reporter gene or a ratio of cells in the CD4⁺ Foxp3⁺ cell group expressing the reporter gene to cells in the CD4⁺ Foxp3⁺ cell group not expressing the reporter gene;
   (V) detecting cells expressing the reporter gene in a CD4⁺ Foxp3⁺ cell group of the non-human mammal which has not been in contact with the test substance, and determining the number of cells in the CD4⁺ Foxp3⁺ cell group expressing the reporter gene or a ratio of cells in the CD4⁺ Foxp3⁺ cell group expressing the reporter gene to cells in the CD4⁺ Foxp3⁺ cell group not expressing the reporter gene; and
   (VI) comparing the number or the ratio determined in (IV) with the number or the ratio determined in (V), and determining, when the number or the ratio determined in (IV) is greater than that determined in (V), that the test substance is a compound that promotes proliferation or accumulation of Treg cells.
(16) A non-human mammal which is used for the method according to (15), and in which the reporter gene is expressed under the control of the IL-10 gene expression.
(17) A method for isolating, from a sample of bacteria belonging to the genus Clostridium, a compound having an activity to promote proliferation or accumulation of regulatory T cells, the method comprising (I) to (III):
   (I) preparing a genomic DNA from the sample of bacteria belonging to the genus Clostridium;
   (II) inserting the genomic DNA into a cloning system, and preparing a gene library derived from the sample of bacteria belonging to the genus Clostridium; and
   (III) isolating a compound having an activity to promote proliferation or accumulation of regulatory T cells, by use of the gene library obtained in step(II).
(18) A method of treatment comprising (I) to (III):
   (I) measuring the percentage and/or absolute amounts of Clostridium Clusters IV and XIV in the microbiota of a subject,
   (II) comparing them to the same measurements in a healthy individual; and
   (III) administering a substance to the subject, if a statistically significant decrease in the number/amounts of Clostridium cluster IV, XIV in the subject compared to the healthy individual is detected, wherein the substance is at least one substance selected from the group consisting of the following (a) to (c) :
   (a) any of the substances described herein;
   (b) an antibiotic against Gram-negative bacteria; and
   (c) the substance selected from the group consisting of almond skin, inulin, oligofructose, raffinose, lactulose, pectin, hemicellulose, amylopectin, acetyl-CoA, biotin, beet molasses, yeast extracts, and resistant starch.
(19) A method of monitoring, comprising (I) to (II):
   (I) measuring level of Clostridium cluster IV, XIV in a subject after administering at least one substance selected from the group consisting of the following (a) to (c) :
   (a) any of the substances described herein;
   (b) an antibiotic against Gram-negative bacteria; and
   (c) the substance selected from the group consisting of almond skin, inulin, oligofructose, raffinose, lactulose, pectin, hemicellulose, amylopectin, acetyl-Co A, biotin, beet molasses, yeast extracts, and resistant starch; and
   (II) if the level increases, it is judged to be a sign that the subject is responding favorably.

### ADVANTAGEOUS EFFECTS OF INVENTION

The compositions of the present invention containing as an active ingredient bacteria belonging to the genus Clostridium or a physiologically active substance derived from the bacteria serves as an excellent composition for inducing the proliferation or accumulation of regulatory T cells (Treg cells). Immunity in a living organism can be suppressed through administration of the composition of the present invention as a pharmaceutical product or ingestion of the composition as a food or beverage. Accordingly, the composition of the present invention can be used, for example, to prevent or treat autoimmune diseases or allergic diseases, as well as to suppress immunological rejection in organ transplantation or the like. In addition, if a food or beverage such as a health food comprises the composition of the present invention, healthy individuals can ingest the composition easily and routinely. As a result, it is possible to induce the proliferation or accumulation of regulatory T cells and thereby to improve immune functions.

### BRIEF DESCRIPTION OF DRAWINGS

FIG. 1 is a schematic diagram showing a method of producing Il10^{venus} mouse.

FIG. 2 is a diagram showing results of Southern blotting performed for analysis as to whether or not the Il10^{venus} mice have an Il10^{venus} allele.

FIG. 3 is a FACS dot-plot diagram showing results obtained when Venus-positive cells and Venus-negative cells from the Il10^{venus} mice were sorted.

FIG. 4 is a graph showing the results obtained when the amounts of IL-10 mRNA expressed in Venus positive-cells and Venus-negative cells of the Il10^{venus} mice were analyzed by real-time RT-PCR.

FIG. 5 is a graph showing change in the ratio of Foxp3⁺ cells in CD4⁺ lymphocytes of SPF mice.

FIG. 6 shows FACS dot-plot diagrams showing analysis results of the ratios of Foxp3⁺ cells in CD4⁺ lymphocytes isolated from the small intestine, the colon, and the peripheral lymph nodes of GF mice and SPF mice.

FIG. 7 is a graph showing analysis results of the ratios of Foxp3⁺ cells in CD4⁺ lymphocytes isolated from the small intestine, the colon, and the peripheral lymph nodes of GF mice and SPF mice.

FIG. 8 shows graphs showing analysis results of the numbers of CD4⁺ Foxp3⁺ cells isolated from the small intestine, the colon, and the peripheral lymph nodes of GF mice and SPF mice.

FIG. 9 is a plot diagram showing analysis results of the ratios of Venus⁺ cells in CD4⁺ cells in various tissues of SPF mice treated with antibiotics.

FIG. 10 shows FACS dot-plot diagrams showing analysis results of the ratio of Foxp3⁺ cell in CD4⁺ lymphocytes isolated from the colonic lamina propria of GF mice to which a fecal suspension of SPF mice was administered.

FIG. 11 is a graph showing analysis results of the ratios of Foxp3⁺ cells in CD4⁺ lymphocytes isolated from the lamina propria of the colon and the lamina propria of the small intestine of GF mice to which a fecal suspension of SPF mice was administered.

FIG. 12 is a graph showing analysis results of the ratio of Foxp3⁺ cells in CD4⁺ lymphocytes isolated from the lamina propria of mice deficient in ILFs, PPs, and colonic-patches.

FIG. 13 shows FACS dot-plot diagrams showing analysis results of the ratios of Foxp3⁺ cells in CD4⁺ lymphocytes isolated from the colonic lamina propria of GF mice to which specific commensal bacteria were administered.

FIG. 14 shows graphs showing analysis results of the ratios of Foxp3⁺ cells in CD4⁺ lymphocytes isolated from the colonic lamina propria of GF mice to which specific commensal bacteria were administered.

FIG. 15 is a graph showing analysis results of the ratios of IFN-γ⁺ cells in CD4⁺ lymphocytes isolated from the colonic lamina propria of mice in which specific commensal bacteria were colonized.

FIG. 16 is a graph showing analysis results of the ratios of IL-17⁺ cells in CD4⁺ lymphocytes isolated from the colonic lamina propria of mice in which specific commensal bacteria were colonized.

FIG. 17 is a graph showing analysis results of the ratios of Foxp3⁺ cells in CD4⁺ lymphocytes isolated from the colon of kinds of SPF mice each being deficient in a pathogen-associated molecular pattern recognition receptor-associated factor.

FIG. 18 is a graph showing analysis results of the ratios of Foxp3⁺ cells in CD4⁺ lymphocytes isolated from the colonic lamina propria of Myd88^{-/-} mice in which the Clostridium was colonized.

FIG. 19 shows FACS dot-plot diagrams showing analysis results of the ratios of Venus⁺ cells in lymphocytes isolated from various tissues of Il10^{venus} mice.

FIG. 20 is a FACS dot-plot diagram showing analysis results of the expression of a T cell receptor β chain on cell surfaces of lymphocytes isolated from the colonic lamina propria of Il10^{venus} mice.

FIG. 21 shows FACS dot-plot diagrams showing analysis results of the expression of IL-17, IL-4, and IFN-γ in lymphocytes isolated from the colonic lamina propria of Il10^{venus} mice.

FIG. 22 shows graphs showing analysis results of the amounts of mRNAs of IL-10, CTLA4, Foxp3, and GITR expressed in spleen Foxp3⁻CD4⁺ cells, spleen Foxp3⁺ CD4⁺ cells, colonic lamina propria Venus⁺ cells, and small intestinal lamina propria Venus⁺ cells.

FIG. 23 shows FACS dot-plot diagrams showing analysis results of the expression of CD4, Foxp3, and Venus in the lamina propria of the small intestine and the lamina propria of the colon of GF Il10^{venus} mice and SPF Il10^{venus} mice.

FIG. 24 shows FACS dot-plot diagrams showing analysis results of the expression of Venus and Foxp3 of CD4 cells in various tissues of SPF Il10^{venus} mice.

FIG. 25 shows FACS dot-plot diagrams showing analysis results of the expression of Foxp3 and Venus in Il10^{venus} mice in which specific commensal bacteria were colonized.

FIG. 26 is a graph showing analysis results of the expression of Foxp3 and/or Venus of CD4⁺ cells in the small intestine of Il10^{venus} mice in which specific commensal bacteria were colonized.

FIG. 27 is a graph showing analysis results of the expression of Foxp3 and/or Venus of CD4⁺ cells in the colon of Il10^{venus} mice in which specific commensal bacteria were colonized.

FIG. 28 is a plot diagram showing analysis results of the ratios of Venus⁺ cells in CD4⁺ cells isolated from various tissues of Il10^{venus} mice treated with antibiotics.

FIG. 29 is a graph showing analysis results of immunoregulatory functions of CD4⁺ Venus⁺ cells from the colonic lamina propria of GF Il10^{venus} mice in which the genus Clostridium was colonized, CD4⁺ Venus⁺ cells from the colonic lamina propria of SPF Il10^{venus} mice, and CD4⁺ GFP⁺ cells from the spleen of Foxp3^{eGFP} reporter mice.

FIG. 30 is a graph showing the results obtained when SPF B6 mice were treated with polymyxin B or vancomycin for 4 weeks, and then analyzed for the ratio of Foxp3⁺ cells in the CD4⁺ cell group.

FIG. 31 is a graph showing the results obtained when SPF mice-derived chloroform-treated feces were orally administered to GF mice, and then the ratio of Foxp3⁺ cells in the CD4⁺ cell group was analyzed.

FIG. 32 is a graph showing the general results of flow cytometry analysis on Helios expression in LP lymphocytes in the thymuses or the colons of SPF mice, GF mice, Lactobacillus-colonized mice, or Clostridium-colonized mice.

FIG. 33 shows plot diagrams showing representative results of flow cytometry analysis on CD4 expression, Foxp3 expression, and Helios expression in the LP lymphocytes in the thymuses or the colons of the SPF mice, the GF mice, the Lactobacillus-colonized mice, or the Clostridium-colonized mice.

FIG. 34 is a graph showing the results obtained when the whole colons derived from GF mice, Lactobacillus-colonized mice, or Clostridium-colonized mice were cultured, and the culture supernatants thereof were analyzed for the TGF-β1 concentration by ELISA.

FIG. 35 is a graph showing the results obtained when intestinal epithelial cells (IECs) derived from GF mice or Clostridium-colonized mice were cultured, and the culture supernatants thereof were analyzed for the TGF-β1 concentration by ELISA.

FIG. 36 is a graph showing the results obtained when splenic CD4⁺ T cells were cultured together with an anti-CD3 antibody and with a culture supernatant of IECs isolated from GF mice or mice colonized with 46 bacterial strains of the genus Clostridium (Clost.) in the presence or absence of an anti-TGF-β antibody, and the T cells were collected on day 5 of the culture and analyzed for Foxp3 expression by real-time RT-PCR.

FIG. 37 is a graph showing the results obtained when C57BL/6 GF mice were orally inoculated with 46 bacterial strains of the genus Clostridium (Clost.) or three bacterial strains of the genus Lactobacillus (Lacto.), and IECs were collected three weeks after the inoculation and analyzed for the relative mRNA expression level of the MMP2 gene by real-time RT-PCR.

FIG. 38 is a graph showing the results obtained when C57BL/6 GF mice were orally inoculated with 46 bacterial strains of the genus Clostridium (Clost.) or three bacterial strains of the genus Lactobacillus (Lacto.), and IECs were collected three weeks after the inoculation and analyzed for the relative mRNA expression level of the MMP9 gene by real-time RT-PCR.

FIG. 39 is a graph showing the results obtained when C57BL/6 GF mice were orally inoculated with 46 bacterial strains of the genus Clostridium (Clost.) or three bacterial strains of the genus Lactobacillus (Lacto.), and IECs were collected three weeks after the inoculation and analyzed for the relative mRNA expression level of the MMP13 gene by real-time RT-PCR.

FIG. 40 is a graph showing the results obtained when C57BL/6 GF mice were orally inoculated with 46 bacterial strains of the genus Clostridium (Clost.) or three bacterial strains of the genus Lactobacillus (Lacto.), and IECs were collected three weeks after the inoculation and analyzed for the relative mRNA expression level of the IDO gene by real-time RT-PCR.

FIG. 41 is a graph showing the results obtained when control mice (SPF) and Clostridium-administered mice (SPF+Clost.) were treated with 2% DSS, observed and measured for the body weight loss, the hardness of stool, and bleeding for six days, and then evaluated numerically.

FIG. 42 is a photograph showing the state of the colons collected on day 6 after the control mice (SPF) and the Clostridium-administered mice (SPF+Clost.) were treated with 2% DSS.

FIG. 43 shows photomicrographs showing the results obtained when the control mice (SPF) and the Clostridium-administered mice (SPF+Clost.) were treated with 2% DSS, and the colons thereof were collected on day 6 and analyzed histologically by HE staining.

FIG. 44 is a graph showing the results obtained when control mice (SPF) and Clostridium-administered mice (SPF+Clost.) were sensitized with oxazolone, and subsequently the inside of each rectum was treated with a 1% oxazolone/50% ethanol solution, and the body weight loss was measured.

FIG. 45 shows photomicrographs showing the results obtained when the control mice (SPF) and the Clostridium-administered mice (SPF+Clost.) were sensitized with oxazolone, and subsequently the inside of each rectum was treated with a 1% oxazolone/50% ethanol solution, and the colons obtained by the treatment were analyzed histologically by HE staining.

FIG. 46 is a graph showing the results obtained when control mice (SPF) and Clostridium-administered mice (SPF+Clost.) were immunized by administering alum-absorbed ovalbumin (OVA) twice at a 2-week interval, and the sera were collected therefrom and analyzed for the concentration of OVA-specific IgE in these sera by ELISA.

FIG. 47 is a graph showing the results obtained when the control mice (SPF) and the Clostridium-administered mice (SPF+Clost.) were immunized by administering the alum-absorbed OVA twice at a 2-week interval, and splenic cells were collected and analyzed for IL-4 production of these splenic cells by in-vitro OVA restimulation.

FIG. 48 is a graph showing the results obtained when the control mice (SPF) and the Clostridium-administered mice (SPF+Clost.) were immunized by administering the alum-absorbed OVA twice at a 2-week interval, and the splenic cells were collected and analyzed for IL-10 production of these splenic cells by the in-vitro OVA restimulation.

FIG. 49 is Phylogenetic tree constructed by the neighbor-joining method with the resulting sequences of the 41 strains of Clostridium and those of known bacteria obtained from Genbank database using Mega software.

FIG. 50 is histograms showing Foxp3 expression gated CD4 cells from GF mice (Germ-free mouse #1 and #2) or GF mice colonized with three strains of Clostridium belonging to cluster IV (3 strains of Clost. mouse #1 and #2).

FIG. 51 is histograms showing Foxp3 expression by CD4 positive lymphocytes from GF mice (GF) or GF mice gavaged with chloroform-treated human stool (GF+Chloro.).

FIG. 52 is a graph showing Foxp3 expression by CD4 positive lymphocytes from GF mice (GF) or GF mice gavaged with chloroform-treated human stool (GF+Chloro.).

FIG. 53 is a graph showing amounts of Clostridium and Bacteroides in feces of mice gavaged with chloroform-treated human stool

### DESCRIPTION OF EMBODIMENTS

Composition Having Effect of Inducing Proliferation or Accumulation of Regulatory T cells

The present invention provides a composition that induces proliferation or accumulation of regulatory T cells, the composition comprising, as an active ingredient, at least one substance selected from the group consisting of the following (a) to (c):
(a) bacteria belonging to the genus Clostridium or a physiologically active substance derived from the bacteria;
(b) a spore-forming fraction of a fecal sample obtained from a mammal or a culture supernatant of the fraction; and
(c) a chloroform-treated fraction of a fecal sample obtained from a mammal or a culture supernatant of the fraction.

In the present invention “regulatory T cells” mean T cells which have a function of suppressing an abnormal or excessive immune response, and which play a role in immune tolerance. The regulatory T cells are typically transcription factor Foxp3-positive CD4-positive T cells. However, the regulatory T cells of the present invention also include transcription factor Foxp3-negative regulatory T cells, as long as the regulatory T cells are IL-10-producing CD4-positive T cells.

The meaning of the “induces proliferation or accumulation of regulatory T cells” in the present invention includes an effect of inducing the differentiation of immature T cells into regulatory T cells, which differentiation leads to the proliferation or the accumulation of regulatory T cells. In addition, the meaning of the “induces proliferation or accumulation of regulatory T cells” in the present invention includes in-vivo effects, in vitro effects, and ex vivo effects. Accordingly, all of the following effects are included: an effect of inducing in vivo proliferation or accumulation of regulatory T cells through administration or ingestion of the bacteria belonging to the genus Clostridium or the physiologically active substance or the like derived from the bacteria; an effect of inducing proliferation or accumulation of cultured regulatory T cells by causing the bacteria belonging to the genus Clostridium or the physiologically active substance or the like derived from the bacteria to act on the cultured regulatory T cells; and an effect of inducing proliferation or accumulation of regulatory T cells which are collected from a living organism and which are intended to be subsequently introduced into a living organism, such as the organism from which they were obtained or another organism, by causing the bacteria belonging to the genus Clostridium or the physiologically active substance or the like derived from the bacteria to act on the regulatory T cells. The effect of inducing proliferation or accumulation of regulatory T cells can be evaluated, for example, as follows. Specifically, the bacteria belonging to the genus Clostridium or the physiologically active substance or the like derived from the bacteria is orally administered to an experimental animal such as a germ-free mouse, then CD4-positive cells in the colon are isolated, and the ratio of regulatory T cells contained in the CD4-positive cells is measured by flow cytometry (refer to Example 7).

The regulatory T cells of which proliferation or accumulation is induced by the composition of the present invention are preferably transcription factor Foxp3-positive regulatory T cells or IL-10-producing regulatory T cells.

The “bacteria belonging to the genus Clostridium,” which are the active ingredient in the composition of the present invention, is not particularly limited as long as the bacteria have the effect of inducing proliferation or accumulation of regulatory T cells. The bacteria preferably belong to the cluster XIVa or the cluster IV. One strain of the bacteria alone can be used for the composition of the present invention, but two or more strains of the bacteria can be used together for the composition of the present invention. The use of multiple strains of bacteria belonging to the cluster XIVa or the cluster IV in combination can bring about an excellent effect on regulatory T cells. In addition to the bacteria belonging to these clusters, bacteria belonging to other clusters (for example, bacteria belonging to the cluster III) can also be used in combination. If more than one strain of bacteria is used (e.g., one or more strain belonging to cluster XIVa, one or more strain belonging to cluster IV, one or more strain belonging to a cluster other than cluster XIVa or cluster IV, such as one or more strain belonging to cluster III), the type and number of strains used can vary widely. The type and number to be used can be determined based on a variety of factors (e.g., the desired effect, such as induction or inhibition of proliferation or accumulation of regulatory T cells; the disease or condition to be treated, prevented or reduced in severity; the age or gender of the recipient) The strains can be present in a single composition, in which case they will be consumed or ingested together, or can be present in more than one composition (e.g., each can be in a separate composition), in which case they can be consumed individually or the compositions can be combined and the resulting combination (combined compositions) consumed or ingested. Any number or combination of strains that proves effective (e.g., any number from one to 200, such as 1 to 100, 1 to 50, 1 to 40, 1 to 30, 1 to 20, 1 to 10, 1 to 5 and any number there between) can be administered. In certain embodiments of the present invention, a combination of some or all of the 46 strains described in Document (Itoh, K., and Mitsuoka, T. Characterization of clostridia isolated from faeces of limited flora mice and their effect on caecal size when associated with germ-free mice. Lab. Animals 19: 111-118 (1985)) is used. For example, at least one, two or more, three, three or more, four, four or more, five, five or more, six, six or more or any other number of the 46 described strains, including 46 strains, can be used. They can be used in combination with one another and in combination with strains not described in the cited reference (e.g., in combination with one or more strains belonging to cluster III). Note that, the cluster of “bacteria belonging to the genus Clostridium” can be identified, for example, as follows. Specifically, the bacteria belonging to the genus Clostridium are classified by PCR using a primer set consisting of SEQ ID NOs 64 and 65 (for Clostridium spp. belonging to the cluster XIVa) or a primer set consisting of SEQ ID NOs 66 and 67 (for Clostridium spp. belonging to the cluster IV) (refer to Example 18). In addition, the bacteria belonging to the genus Clostridium are classified by sequencing of 16S rRNA gene amplified using a primer set consisting of SEQ ID NOs 19 and 20 (refer to Example 7).

Viable cells of the bacteria belonging to the genus Clostridium can be used for the composition of the present invention, and killed cells thereof may also be used for the composition. In addition, from the viewpoint of stability to heat, resistance to antibiotics and the like, and long storage period, the bacteria belonging to the genus Clostridium are preferably in the form of spore.

The meaning of the “physiologically active substance derived from bacteria belonging to the genus Clostridium” of the present invention includes substances contained in the bacteria, secretion products of the bacteria, and metabolites of the bacteria. Such a physiologically active substance can be identified by purifying an active component from the bacteria, a culture supernatant thereof, or intestinal tract contents in the intestinal tract of a mouse in which only bacteria belonging to the genus Clostridium are colonized by an already known purification method.

The active ingredient “spore-forming fraction of a fecal sample obtained from a mammal” in the composition of the present invention is not particularly limited, as long as the fraction includes spore-forming bacteria present in feces of a mammal, and has the effect of inducing proliferation or accumulation of regulatory T cells.

The active ingredient “chloroform-treated fraction of a fecal sample obtained from a mammal” in the composition of the present invention is not particularly limited, as long as the fraction is obtained by treating feces of a mammal with chloroform (for example, 3% chloroform), and has the effect of inducing proliferation or accumulation of regulatory T cells.

Note that the “mammal” in the present invention is not particularly limited, and examples thereof include humans, mice, rats, cattle, horses, pigs, sheep, monkeys, dogs, and cats.

Meanwhile, when the “spore-forming fraction of a fecal sample obtained from a mammal” or the “chloroform-treated fraction of a fecal sample obtained from a mammal” is cultured in a medium, substances contained in the bacteria, secretion products of the bacteria, metabolites of the bacteria are released from the bacteria and the like contained in the fraction. The meaning of the active ingredient “culture supernatant of the fraction” in the composition of the present invention includes such substances, secretion products, and metabolites. The culture supernatant is not particularly limited, as long as the culture supernatant has the effect of inducing proliferation or accumulation of regulatory T cells. Examples of the culture supernatant include a protein fraction of the culture supernatant, a polysaccharide fraction of the culture supernatant, a lipid fraction of the culture supernatant, and a low-molecular weight metabolite fraction of the culture supernatant.

The composition of the present invention may be in the form of a pharmaceutical composition, a food or beverage (which may also be an animal feed), or a reagent used for an animal model experiment, the pharmaceutical composition, the food or beverage, and the reagent having the effect of inducing proliferation or accumulation of regulatory T cells. An example of the present invention revealed that regulatory T cells (Treg cells) induced by bacteria or the like belonging to the genus Clostridium suppressed the proliferation of effector T-cells. Accordingly, the composition of the present invention can be used suitably as a composition having an immunosuppressive effect. The immunosuppressive effect can be evaluated, for example, as follows. Specifically, regulatory T cells isolated from an experimental animal, such as a mouse, to which the composition of the present invention is orally administered are caused to act on effector T-cells (CD4⁺ CD25⁻ cells) isolated from the spleen, and then proliferation ability thereof is measured by using the intake amount of [³H]-thymidine as an index (refer to Example 14).

The composition of the present invention can be used, for example, as a pharmaceutical composition for preventing or treating an autoimmune disease such as chronic inflammatory bowel disease, systemic lupus erythematosus, rheumatoid arthritis, multiple sclerosis, or Hashimoto's disease, or an allergic disease such as pollenosis or asthma; a pharmaceutical composition for suppressing rejection in organ transplantation or the like; a food or beverage for improving immune functions; or a reagent for suppressing the proliferation or function of effector T-cells.

More specific examples of target diseases of the composition of the present invention include autoimmune diseases, allergic diseases, and rejection in organ transplantations and the like, such as inflammatory bowel disease (IBD), ulcerative colitis, Crohn's disease, sprue, autoimmune arthritis, rheumatoid arthritis, Type I diabetes, multiple sclerosis, graft vs. host disease following bone marrow transplantation, osteoarthritis, juvenile chronic arthritis, Lyme arthritis, psoriatic arthritis, reactive arthritis, spondy loarthropathy, systemic lupus erythematosus, insulin dependent diabetes mellitus, thyroiditis,. asthma, psoriasis, dermatitis scleroderma, atopic dermatitis, graft versus host disease, acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlejn purpurea, microscopic vasculitis of the kidneys, chronic active hepatitis, uveitis, septic shock, toxic shock syndrome, sepsis syndrome, cachexia, acquired immunodeficiency syndrome, acute transverse myelitis, Huntington's chorea, Parkinson's disease, Alzheimer's disease, stroke, primary biliary cirrhosis, hemolytic anemia, polyglandular deficiency type I syndrome and polyglandular deficiency type II syndrome, Schmidt's syndrorme, adult (acute) respiratory distress syndrome, alopecia, alopecia areata, seronegative arthopathy, arthropathy, Reiter's disease, psoriatic arthropathy, chlamydia, yersinia and salmonella associated arthropathy, spondyloarhopathy, atheromatous disease/arteriosclerosis, atopic allergy, food allergies, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis C, common varied immunodeficiency (common variable hypogammaglobulinaemia), dilated cardiomyopathy, fibrotic lung disease, cryptogenic fibrosing alveolitis, postinflammatory interstitial lung disease, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polymyositis associated lung disease, Sjogren's disease associated lung disease, ankylosing spondy litis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-1 autoimmune hepatitis (classical autoimmune or lupoid hepatitis), type-2 autoimmune hepatitis (anti-LKM antibody hepatitis), autoimmune mediated hypoglycemia, type B insulin resistance with acanthosis nigricans, hypoparathyroidism, acute immune disease associated with organ transplantation, chronic immune disease associated with organ transplantation, osteoarthrosis, primary sclerosing cholangitis, idiopathic leucopenia, autoimmune neutropenia, renal disease NOS, glomerulonephritides, microscopic vasulitis of the kidneys, discoid lupus, erythematosus, male infertility idiopathic or NOS, sperm autoimmunity, multiple sclerosis (all subtypes), insulindependent diabetes mellitus, sympathetic ophthalmia, pulmonary hypertension secondary to connective tissue disease, Goodpasture's syndrome, pulmonary manifestation of polyarteritis nodosa, acute rheumatoid fever, rheumatoid spondylitis, Still's disease, systemic sclerosis, Takayasu's disease/arteritis, autoimmune thrombocytopenia, idiopathic thrombocytopenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo, allergic rhinitis (pollen allergies), anaphylaxis, pet allergies, latex allergies, drug allergies, allergic rhinoconjuctivitis, eosinophilic esophagitis, hypereosinophilic syndrome, eosinophilic gastroenteritis cutaneous lupus erythematosus, eosinophilic esophagitis, hypereosinophilic syndrome, and eosinophilic gastroenteritis.

The composition of the present invention can also be used as a pharmaceutical composition for preventing or treating infectious diseases in an individual whose resistance to the infectious diseases is impaired because of damage due to excessive inflammation caused by the immunity.

Example of infectious pathogens which impair maintenance or recovery of homeostasis of a host, and which eventually bring about such immunopathological tissue damage include Salmonella, Shigella, Clostridium difficile, Mycobacterium (which cause the disease tuberculosis), protozoa (which cause the disease malaria), filarial nematodes (which cause the disease filariasis), Schistosoma (which cause the disease schistosomiasis), Toxoplasma (which cause the disease toxoplasmosis), Leishmania (which cause the disease leishmaniasis), HCV and HBV (which cause the disease hepatitis C and hepatitis B), and herpes simplex viruses (which cause the disease herpes).

Pharmaceutical preparations can be formulated from the composition of the present invention by already known drug formulation methods. For example, the composition of the present invention can be used orally or parenterally in the forms of capsules, tablets, pills, liquids, powders, granules, fine granules, film-coated preparations, pellets, troches, sublingual preparations, chewables, buccal preparations, pastes, syrups, suspensions, elixirs, emulsions, liniments, ointments, plasters, cataplasms, transdermal absorption systems, lotions, inhalations, aerosols, injections, suppositories, and the like.

For formulating these preparations, the composition of the present invention can be used in appropriate combination with carriers acceptable pharmacologically or acceptable for a food or beverage, specifically, with sterile water, physiological saline, vegetable oil, solvent, a base material, an emulsifier, a suspending agent, a surfactant, a stabilizer, a flavoring agent, an aromatic, an excipient, a vehicle, a preservative, a binder, a diluent, a tonicity adjusting agent, a soothing agent, a bulking agent, a disintegrating agent, a buffer agent, a coating agent, a lubricant, a colorant, a sweetener, a thickening agent, a flavor corrigent, a solubilizer, other additives, or the like.

Meanwhile, for formulating a pharmaceutical preparation thereof, and particularly for formulating a pharmaceutical preparation for oral administration, it is preferable to use in combination a composition which enables an efficient delivery of the composition of the present invention to the colon, from the viewpoint of more efficiently inducing the proliferation or accumulation of regulatory T cells in the colon.

Such a composition or method which enables the delivery to the colon is not particularly limited, and known compositions or methods can be employed as appropriate. Examples thereof include pH sensitive compositions, more specifically, enteric polymers which release their contents when the pH becomes alkaline after the enteric polymers pass through the stomach. When a pH sensitive composition is used for formulating the pharmaceutical preparation, the pH sensitive composition is preferably a polymer whose pH threshold of the decomposition of the composition is 6.8 to 7.5. Such a numeric value range is a range where the pH shifts toward the alkaline side at a distal portion of the stomach, and hence is a suitable range for use in the delivery to the colon.

Moreover, another example of the composition enabling the delivery to the colon is a composition which ensures the delivery to the colon by delaying the release of the contents by approximately 3 to 5 hours, which corresponds to the small intestinal transit time. In an example of formulating a pharmaceutical preparation using the composition for delaying the release, a hydrogel is used as a shell. The hydrogel is hydrated and swells upon contact with gastrointestinal fluid, so that the contents are effectively released. Furthermore the delayed release dosage units include drug-containing compositions having a material which coats or selectively coats a drug. Examples of such a selective coating material include in vivo degradable polymers, gradually hydrolyzable polymers, gradually water-soluble polymers, and/or enzyme degradable polymers. A preferred coating material for efficiently delaying the release is not particularly limited, and examples thereof include cellulose-based polymers such as hydroxypropyl cellulose, acrylic acid polymers and copolymers such as methacrylic acid polymers and copolymers, and vinyl polymers and copolymers such as polyvinylpyrrolidone.

Examples of the composition enabling the delivery to the colon further include bioadhesive compositions which specifically adhere to the colonic mucosal membrane (for example, a polymer described in the specification of U.S. Pat. No. 6,368,586), and compositions into which a protease inhibitor is incorporated for protecting particularly a biopharmaceutical preparation in the gastrointestinal tracts from decomposition due to an activity of a protease.

An example of a system enabling the delivery to the colon is a system of delivering a composition to the colon by pressure change in such a way that the contents are released by utilizing pressure change caused by generation of gas in bacterial fermentation at a distal portion of the stomach. Such a system is not particularly limited, and a more specific example thereof is a capsule which has contents dispersed in a suppository base and which is coated with a hydrophobic polymer (for example, ethyl cellulose).

Another example of the system enabling the delivery to the colon is a system of delivering a composition to the colon, the system being specifically decomposed by an enzyme (for example, a carbohydrate hydrolase or a carbohydrate reductase) present in the colon. Such a system is not particularly limited, and more specific examples thereof include systems which use food components such as non-starch polysaccharides, amylose, xanthan gum, and azopolymers.

When used as a pharmaceutical composition, the composition of the present invention may be used in combination with an already known pharmaceutical composition for use in immunosuppression. Such a known pharmaceutical composition is not particularly limited, and may be at least one therapeutic composition selected from the group consisting of corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, anti-cholinergic decongestants, mast-cell stabilizers, monoclonal anti-IgE antibodies, vaccines (preferably vaccines used for vaccination where the amount of an allergen is gradually increased), and combinations thereof. It is preferable to use these therapeutic compositions in combination with the composition of the present invention.

When the composition of the present invention is used as a food or beverage, the food or beverage can be, for example, a health food, a functional food, a food for specified health use, a dietary supplement, a food for patients, or an animal feed. The food or beverage of the present invention can be ingested in the forms of the compositions as described above, and also can be ingested in the forms of various foods and beverages. Specific examples of the foods and beverages include various beverages such as juices, refreshing beverages, tea beverages, drink preparations, jelly beverages, and functional beverages; alcoholic beverages such as beers; carbohydrate-containing foods such as rice food products, noodles, breads, and pastas; paste products such as fish hams, sausages, paste products of seafood; retort pouch products such as curries, food dressed with a thick starchy sauces, and Chinese soups; soups; dairy products such as milk, dairy beverages, ice creams, cheeses, and yogurts; fermented products such as fermented soybean pastes, yogurts, fermented beverages, and pickles; bean products; various confectionery products such as Western confectionery products including biscuits, cookies, and the like, Japanese confectionery products including steamed bean-jam buns, soft adzuki-bean jellies, and the like, candies, chewing gums, gummies, cold desserts including jellies, crème caramels, and frozen desserts; instant foods such as instant soups and instant soy-bean soups; microwavable foods; and the like. Further, the examples also include health foods and beverages prepared in the forms of powders, granules, tablets, capsules, liquids, pastes, and jellies. The composition of the present invention can be used for animals including humans. The animals, other than humans, are not particularly limited, and the composition can be used for various livestock, poultry, pets, experimental animals, and the like. Specific examples of the animals include pigs, cattle, horses, sheep, goats, chickens, wild ducks, ostriches, domestic ducks, dogs, cats, rabbits, hamsters, mice, rats, monkeys, and the like, but the animals are not limited thereto.

Without wishing to be bound by theory, in the present invention, individuals in which the relative abundance of bacteria belonging to the group Firmicutes (the group to which the Clostridium clusters IV and XIVa belong) is large gain more body weight than individuals in which the relative abundance of bacteria belonging to the group Bacteroidetes is large. Accordingly, the composition of the present invention is capable of conditioning absorption of nutrients and improving feed efficiency. From such a viewpoint, the composition of the present invention can be used for promoting body weight gain, or for an animal feed good in feed efficiency.

Moreover, the addition of the composition of the present invention to an antibiotic-free animal feed makes it possible to increase the body weight of a subject that ingests the animal feed to a level equal to or higher than those achieved by antibiotic-containing animal feeds, and also makes it possible to reduce pathogenic bacteria in the stomach to a level equal to those achieved by typical antibiotic-containing animal feeds. Accordingly, the composition of the present invention can be used for an animal feed which does not need the addition of antibiotics.

In addition, unlike conventional bacteria (Lactobacillus and Bifidobacteria) in commercial use which are not easy to incorporate into the livestock production, the composition of the present invention in the spore form can be pelletized, sprayed, or easily mixed with an animal feed, and also can be added to drinking water.

The feeding of such an animal feed using the composition of the present invention is not particularly limited, and the animal feed may be fed to a subject at regular intervals in a selective manner, or may be fed for a certain period (for example, at its birth, during weaning, or when the subject to be fed is relocated or shipped).

Moreover, from the above-described viewpoint, the composition of the present invention can be preferably used for malnourished humans. In other words, also when the subject who ingests the composition is a human, the composition of the present invention can preferably be used for promoting the body weight gain, and enhancing the energy absorption from foods.

The food or beverage of the present invention can be manufactured by a manufacturing technique which is well known in the technical field. To the food or beverage, one or more components (for example, a nutrient) which are effective for the improvement of an immune function by the immunosuppressive effect may be added. In addition, the food or beverage may be combined with another component or another functional food exhibiting a function other than the function of the improvement of an immune function to thereby serve as a multi-functional food or beverage.

Moreover, the composition of the present invention can be preferably incorporated into foods requiring a processing step which may destroy ordinary probiotic strains. Specifically, most commercially usable probiotic strains cannot be incorporated into foods which need to be processed by any one of a heat treatment, long term storage, a freezing treatment, a mechanical stress treatment, and a high-pressure treatment (for example, extrusion forming or roll forming). On the other hand, because of an advantageous nature of forming spores, the composition of the present invention can be easily incorporated into such processed foods.

For example, the composition of the present invention in the form of spore can survive even in a dried food, and can remain living even after being ingested. Likewise, the composition of the present invention can withstand low-temperature sterilization processes, typically processes at a temperature in a range from 70° C. to the boiling point, both inclusive. Thus, the composition of the present invention can be incorporated into all kinds of dairy products. Furthermore, the composition of the present invention can withstand long-term storage of many years; high-temperature processing such as baking and boiling; low-temperature processing such as freezing and cold storage; and high-pressure treatments such as extrusion forming and roll forming.

The foods which need to be processed under such harsh conditions are not particularly limited, and examples thereof include foods which need to be processed in a microwave oven to be edible (for example, oatmeal), foods which need to be baked to be edible (for example, muffin), foods which need to be subjected to a sterilization high-temperature treatment for a short period of time to be edible (for example, milk), and foods which need to be heated to be drinkable (for example, hot tea).

When the composition of the present invention is administered or ingested, the amount thereof for the administration or ingestion is selected as appropriate depending on the age, body weight, symptoms, health conditions, of a subject, the kind of the composition (a pharmaceutical product, a food or beverage, or the like), and the like. For example, the amount per administration or ingestion is generally 0.01 mg/kg body weight to 100 mg/kg body weight, and preferably 1 mg/kg body weight to 10 mg/kg body weight. The present invention also provides a method for suppressing the immunity of a subject, the method being characterized in that the bacteria belonging to the genus Clostridium or the physiologically active substance derived from the bacteria is administered into or ingested by the subject as described above.

A product of the composition of the present invention (a pharmaceutical product, a food or beverage, or a reagent) or a manual thereof may be provided with a note stating that the product can be used to suppress the immunity (including a note stating that the product has an immunosuppressive effect, and a note stating that the product has an effect of suppressing the proliferation or function of effector T-cells). Here, the “provision to the product or the manual thereof with the note” means that the note is provided to a main body, a container, a package, or the like of the product, or the note is provided to a manual, a package insert, a leaflet, or other printed matters, which disclose information on the product.

### Method for Inducing Proliferation or Accumulation of Regulatory T Cells

As described above, and as will be shown in Examples, the administration of the composition of the present invention to an individual makes it possible to induce proliferation or accumulation of regulatory T cells in the individual. Thus, the present invention can provides a method for inducing proliferation or accumulation of regulatory T cells in an individual, the method comprising a step of administering, to the individual, at least one substance selected from the group consisting of the following (a) to (c):
(a) bacteria belonging to the genus Clostridium or a physiologically active substance derived from the bacteria;
(b) a spore-forming fraction of a fecal sample obtained from a mammal or a culture supernatant of the fraction; and
(c) a chloroform-treated fraction of a fecal sample obtained from a mammal or a culture supernatant of the fraction.

Note that, the “individual” in the present invention is not particularly limited, and examples thereof include humans, various kinds of livestock, poultry, pets, experimental animals, and the like. The “individual” may be in a healthy state or a diseased state.

Moreover, as will be shown in Example 5 to be described later, Gram-positive commensal bacteria play principal roles in the proliferation or accumulation of regulatory T cells. Accordingly, the present invention can also provide a method for inducing proliferation or accumulation of regulatory T cells in an individual, the method comprising a step of administering an antibiotic against Gram-negative bacteria to the individual.

In the present invention, the “antibiotic against Gram-negative bacteria” is not particularly limited, and examples thereof include aminoglycoside antibiotics (amikacin, gentamicin, kanamycin, neomycin, netilmicin, tobramycin, and paromomycin), cephalosporin antibiotics (cefaclor, cefamandole, cefoxitin, cefprozil, cefuroxime, cefixime, cefdinir, cefditoren, cefoperazone, cefotaxime, ceftazidime, ceftibuten, ceftizoxime, ceftriaxone, and cefoxotin), sulfonamides, ampicillin, and streptomycin. Without wishing to be bound by theory, the “antibiotic against Gram-negative bacteria” according to the present invention is preferably one which reduces Gram-negative bacteria, and contributes to the colonization of Gram-positive bacteria.

Moreover, a prebiotic composition such as almond skin, inulin, oligofructose, raffinose, lactulose, pectin, hemicellulose (such as xyloglucan and alpha-glucans), amylopectin, and resistant starch which are not decomposed in the upper gastrointestinal tract and promote the growth of intestinal microbes in the intestinal tract, as well as growth factors such as acetyl-Co A, biotin, beet molasses, and yeast extracts, contribute to the proliferation of bacteria belonging to the genus Clostridium. Accordingly, the present invention can also provide a method for inducing proliferation or accumulation of regulatory T cells in an individual, the method comprising a step of administering, to the individual, at least one substance selected from the group consisting of these substances.

Meanwhile, in the “method for inducing proliferation or accumulation of regulatory T cells” of the present invention, the composition of the present invention, the above-described “antibiotic against Gram-negative bacteria,” and the above-described “prebiotic composition or growth factor” may be used in combination. Such combined use is not particularly limited, and examples of the combined use are as follows: the “antibiotic against Gram-negative bacteria” is administered to an individual in advance, and then the composition of the present invention is administered; the “antibiotic against Gram-negative bacteria” and the composition of the present invention are simultaneously administered to an individual; the “prebiotic composition or growth factor” is administered to an individual in advance, and then the composition of the present invention is administered; the “prebiotic composition or growth factor” and the composition of the present invention are simultaneously administered to an individual; the composition of the present invention, the “antibiotic against Gram-negative bacteria,” and the “prebiotic composition or growth factor” are administered to an individual simultaneously or individually at any appropriate time.

Moreover, a therapeutic composition may be administered to an individual together with at least one substance selected from the group consisting of the composition of the present invention, the “antibiotic against Gram-negative bacteria,” and the “prebiotic composition or growth factor.”

Such a therapeutic composition is not particularly limited, and may be at least one therapeutic composition selected from the group consisting of corticosteroids, mesalazine, mesalamine, sulfasalazine, sulfasalazine derivatives, immunosuppressive drugs, cyclosporin A, mercaptopurine, azathiopurine, prednisone, methotrexate, antihistamines, glucocorticoids, epinephrine, theophylline, cromolyn sodium, anti-leukotrienes, anti-cholinergic drugs for rhinitis, anti-cholinergic decongestants, mast-cell stabilizers, monoclonal anti-IgE antibodies, vaccines (preferably, vaccines used for vaccination where the amount of an allergen is gradually increased), and combinations thereof. It is preferable to use these therapeutic compositions in combination with the above-described substance.

Moreover, there is no particular limitation imposed on the combined use of the therapeutic composition with at least one substance selected from the group consisting of the composition of the present invention, the “antibiotic against Gram-negative bacteria,” and the “prebiotic composition or growth factor”. For example, the “one substance” and the therapeutic composition are administered orally or parenterally to an individual simultaneously or individually at any appropriate time.

Moreover, in the above-described “method for inducing proliferation or accumulation of regulatory T cells,” whether or not the administration of the composition of the present invention or the like actually induces the proliferation or accumulation of regulatory T cells can be determined by using, as an index, increase or reinforcement of at least one selected from the group consisting of the number of regulatory T cells, the ratio of regulatory T cells in the T cell group of the colon, a function of regulatory T cells, and expression of a marker of regulatory T cells. It is preferable to use one measurement selected from the group consisting of promotion of IL-10 expression, promotion of CTLA4 expression, promotion of IDO expression, and suppression of IL-4 expression, as the index of the induction of proliferation or accumulation of regulatory T cells.

Note that examples of a method for detecting such expression include the northern blotting, the RT-PCR, and the dot blotting for detection of gene expression at the transcription level; and the ELISA, the radioimmunoassay, the immunoblotting, the immunoprecipitation, and the flow cytometry for detection of gene expression at the translation level.

Meanwhile, a sample used for measuring such an index is not particularly limited, and examples thereof include blood sampled from an individual and tissue pieces obtained in a biopsy.

### Method for Predicting Response of Individual to Composition of Present Invention and/or Prognosis of Individual

The present invention can provide a method in which the absolute amount or the ratio of bacteria belonging to the genus Clostridium in a microbiota of an individual is determined, and, when the ratio or the absolute value of the bacteria belonging to the genus Clostridium is reduced in comparison with a base line value obtained by performing a similar determination on an individual in a typical health state, it is determined that the individual is possibly responsive to the composition of the present invention.

In one embodiment, a method to predict a subject's response to a substance and/or the subject's prognosis is provided. The method comprises measuring the percentage or absolute amounts of Clostridium clusters IV and XIV in the microbiota of the subject and comparing them to a baseline value of the same measurements in a prototypical healthy subject, wherein a decreased absolute amount or percentage level of Clostridium clusters IV and/or XIV indicates that the subject may respond favorably to the compositions of the invention.

In one embodiment, the method further comprises measuring the composition of the microbiota of the subject after administration of the substance, wherein an increase in the percentage or absolute number of Clostridium spp. belonging to clusters IV, XIV after administration of the compositions of the present invention relative to prior to the administering is a positive indicator of enhanced immunosuppression (or immunoregulation). The measurement of the composition of the subject's microbiota can be made with techniques known in the art, such as 16srRNA sequencing.

Note that, in these embodiments, the substance is at least one substance selected from the group consisting of the following (a) to (e) :
(a) bacteria belonging to the genus Clostridium or a physiologically active substance derived from the bacteria;
(b) a spore-forming fraction of a fecal sample obtained from a mammal or a culture supernatant of the fraction;
(c) a chloroform-treated fraction of a fecal sample obtained from a mammal or a culture supernatant of the fraction;
(d) an antibiotic against Gram-negative bacteria according to the present invention; and
(e) at least one substance selected from the group consisting of almond skin, inulin, oligofructose, raffinose, lactulose, pectin, hemicellulose (such as xyloglucan and alpha-glucans), amylopectin, acetyl-Co A, biotin, beet molasses, yeast extracts, and resistant starch.

### Method for Inhibiting Proliferation or Accumulation of Regulatory T Cells

As will be shown in Example 5 to be described later, Gram-positive commensal bacteria have principal roles in the proliferation or accumulation of regulatory T cells. Accordingly, the present invention can also provide a method for inhibiting proliferation or accumulation of regulatory T cells in an individual, the method comprising a step of administering an antibiotic against Gram-positive bacteria to the individual.

In the present invention, the “antibiotic against Gram-positive bacteria” is not particularly limited, and examples thereof include cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); and carbapenem antibiotics (ertapenem, doripenem, imipenem/cilastatin, and meropenem).

As described above, the “individual” in the present invention is not particularly limited, and examples thereof include humans, various kinds of livestock, poultry, pets, experimental animals, and the like. The “individual” may be in a healthy state or a diseased state. Such a diseased state is not particularly limited, and examples thereof include states of being subjected to cancer immunotherapy and of suffering from an infectious disease.

Moreover, as another mode of the “method for inhibiting proliferation or accumulation of regulatory T cells,” the present invention can provide a method for inhibiting proliferation or accumulation of regulatory T cells in an individual, the method comprising a step of administering, to the individual, any one of an antibody, an antibody fragment, and a peptide, which are against an antigen that is at least one substance selected from the group consisting of the following (a) to (c):
(a) bacteria belonging to the genus Clostridium or a physiologically active substance derived from the bacteria;
(b) a spore-forming fraction of a fecal sample obtained from a mammal or a culture supernatant of the fraction; and
(c) a chloroform-treated fraction of a fecal sample obtained from a mammal or a culture supernatant of the fraction.

### Vaccine Composition and Method for Treating or Preventing Infectious Disease or Autoimmune Disease by Using the Vaccine Composition

As described above, and as will be shown in Example 15 to be described later, the induction of Treg cells in the colon by the Clostridium has an important role in local and systemic immune responses. Accordingly, the present invention can also provide a “vaccine composition comprising at least one substance selected from the group consisting of the following (a) to (c): (a) bacteria belonging to the genus Clostridium; (b) a spore of bacteria in a spore-forming fraction of a fecal sample obtained from a mammal; and (c) bacteria in a chloroform-treated fraction of a fecal sample obtained from a mammal” and a “method for treating, aiding in treating, reducing the severity of, or preventing at least one disease selected from infectious diseases and autoimmune diseases in an individual, the method comprising administering the vaccine composition to the individual.”

Note that such “autoimmune diseases” are not particularly limited, and examples thereof include those described as the “specific examples of target diseases” in Composition Having Effect of Inducing Proliferation or Accumulation of Regulatory T cells. The “infectious diseases” are also not particularly limited, and examples thereof include infectious diseases associated with “infectious pathogens” described as the “example of infectious pathogens” in Composition Having Effect of Inducing Proliferation or Accumulation of Regulatory T cells.

### Method for Screening for Compound Having Activity to Promote Proliferation or Accumulation of Regulatory T Cells

The present invention can also provide a method for screening for a compound having an activity to promote proliferation or accumulation of regulatory T cells, the method comprising:
(1) preparing a test substance from at least one substance selected from the group consisting of the following (a) to (c):
   (a) bacteria belonging to the genus Clostridium or a physiologically active substance derived from the bacteria;
   (b) a spore-forming fraction of a fecal sample obtained from a mammal or a culture supernatant of the fraction; and
   (c) a chloroform-treated fraction of a fecal sample obtained from a mammal or a culture supernatant of the fraction.
(2) preparing non-human mammals in which a reporter gene is to be expressed under control of IL-10 gene expression;
(3) bringing the test substance into contact with the non-human mammal;
(4) after the contact with the test substance, detecting cells expressing the reporter gene in a CD4⁺ Foxp3⁺ cell group of the non-human mammal, and determining the number of cells in the CD4⁺ Foxp3⁺ cell group expressing the reporter gene or a ratio of cells in the CD4⁺ Foxp3⁺ cell group expressing the reporter gene to cells in the CD4⁺ Foxp3⁺ cell group not expressing the reporter gene;
(5) detecting cells expressing the reporter gene in a CD4⁺ Foxp3⁺ cell group of the non-human mammal which has not been in contact with the test substance, and determining the number of cells in the CD4⁺ Foxp3⁺ cell group expressing the reporter gene or a ratio of cells in the CD4⁺ Foxp3⁺ cell group expressing the reporter gene to cells in the CD4⁺ Foxp3⁺ cell group not expressing the reporter gene; and
(6) comparing the absolute numbers or the ratios determined in steps (4) with the number or the ratio determined in (5), and determining, when the number or the ratio determined in (4) is greater than that determined in (5), that the test substance is a compound that promotes proliferation or accumulation of Treg cells.

The “test substance” according to the present invention is not particularly limited, as long as the test substance is a substance prepared from at least one substance selected from the group consisting of the substances (a) to (c). Examples of the test substance include proteins, polysaccharides, lipids, and nucleic acids which are derived from at least one substance selected from the group consisting of the above described substances (a) to (c)

The “non-human mammal in which a reporter gene is to be expressed under control of IL-10 gene expression” according to the present invention is not particularly limited, as long as the non-human mammal is a non-human mammal having a reporter gene whose expression is controlled by an IL-10 gene expression control region (for example, a promoter, or an enhancer). Examples of such a reporter gene include genes encoding fluorescent proteins (for example, GFP), and genes encoding luciferase. As the “non-human mammal in which a reporter gene is to be expressed under control of IL-10 gene expression” according to the present invention, an Il10^{venus} mouse to be shown later in Examples can be preferably used.

The “contact” according to the present invention is not particularly limited, and examples thereof include administration of the test substance to the non-human mammal orally or parenterally (for example, intraperitoneal injection, or intravenous injection).

The present invention can also provide a non-human mammal which is used for the method, and in which the reporter gene is to be expressed under the control of the IL-10 gene expression.

Furthermore, the present invention can also provide a method for isolating, from a sample of bacteria belonging to the genus Clostridium, a compound having an activity to promote proliferation or accumulation of regulatory T cells, the method comprising the following steps (1) to (3):
(1) preparing a genomic DNA from the sample of bacteria belonging to the genus Clostridium;
(2) inserting the genomic DNA into a cloning system, and preparing a gene library derived from the sample of bacteria belonging to the genus Clostridium; and
(3) isolating a compound having an activity to promote proliferation or accumulation of regulatory T cells, by use of the gene library obtained in step (2).

In such steps, methods for the preparation and the isolation are not particularly limited, and known techniques for an in-vitro or in-vivo system can be used as appropriate. Moreover, the compound isolated by this method is not particularly limited, and examples thereof include nucleic acids (for example, a DNA, a mRNA, and a rRNA) derived from bacteria belonging to the genus Clostridium, as well as polypeptides and proteins derived from the bacteria belonging to the genus Clostridium.

### Other Embodiment Modes according to Present Invention

In addition to the above-described embodiment modes, the present invention can also provide the following embodiment modes.

Specifically, the present invention can also provide a method for determining the composition of a microbiota in an individual, wherein the increase in the ratio or the absolute number of bacteria belonging to the genus Clostridium after the administration of the composition of the present invention to the individual with respect to the ratio or the absolute number before the administration is used as an index of increased immunosuppression. In such a method, the method for determining the composition of the microbiota is not particularly limited, and known techniques (for example, 16S rRNA sequencing) can be used as appropriate.

The present invention can also provide a method for measuring differentiation of Treg cells, wherein the increase in differentiation of Treg cells in an individual after administration of the composition of the present invention to the individual with respect to that before the administration is used as an index of increased immunosuppression (or immunoregulation).

Moreover, the composition of the present invention can also be administered to an individual under an antibiotic treatment. The timing of the administration is not particularly limited, and the composition of the present invention can be administered before or simultaneously with the antibiotic treatment, for example. Meanwhile, the composition of the present invention is preferably administered in the spore form from the viewpoint of resistance to antibiotics.

Moreover, in a preferred mode of such administration, the composition of the present invention is administered after or simultaneously with administration of an antibiotic against Gram-positive bacteria, for example. Note that such an “antibiotic against Gram-positive bacteria” is not particularly limited, and examples thereof include cephalosporin antibiotics (cephalexin, cefuroxime, cefadroxil, cefazolin, cephalothin, cefaclor, cefamandole, cefoxitin, cefprozil, and ceftobiprole); fluoroquinolone antibiotics (cipro, Levaquin, floxin, tequin, avelox, and norflox); tetracycline antibiotics (tetracycline, minocycline, oxytetracycline, and doxycycline); penicillin antibiotics (amoxicillin, ampicillin, penicillin V, dicloxacillin, carbenicillin, vancomycin, and methicillin); and carbapenem antibiotics (ertapenem, doripenem, imipenem/cilastatin, and meropenem).

Meanwhile, in another preferred mode of such administration, the composition of the present invention is administered after (or simultaneously with) a treatment using vancomycin, metronidazole, linezolid, ramoplanin, or fidaxomicin, for example.

### Examples

Hereinafter, the present invention is described more specifically on the basis of Examples. However, the present invention is not limited to Examples below.

Note that mice used in Examples were prepared or produced as follows. In the following description, mice may be referred to with “SPF” or “GF” attached in front thereof. These “SPF” and “GF” indicate that the mice were maintained in the absence of specific pathogenic bacteria (specific pathogen-free, SPF), and that the mice were maintained under Germ-Free (GF) conditions, respectively.

### Mice

C57BL/6, Balb/c, and IQI mice maintained under SPF or GF conditions were purchased from Sankyo Labo Service Corporation, Inc. (Japan), JAPAN SLC, INC. (Japan), CLEA Japan, Inc. (Japan), or The Jackson Laboratory (USA). GF mice and gnotobiotic mice were bread and maintained within the gnotobiotic facility of The University of Tokyo, Yakult Central Institute for Microbiological Research, or Sankyo Labo Service Corporation, Inc. Myd88^{-/-}, Rip2^{-/-}, and Card9^{-/-} mice were produced as described in Non-Patent Documents 1 to 3, and backcrossed for 8 generations or more, so that a C57BL/6 genetic background was achieved. Foxp3^{eGFP} mice were purchased from the Jackson Laboratory.

### Il10^{venus} Mice

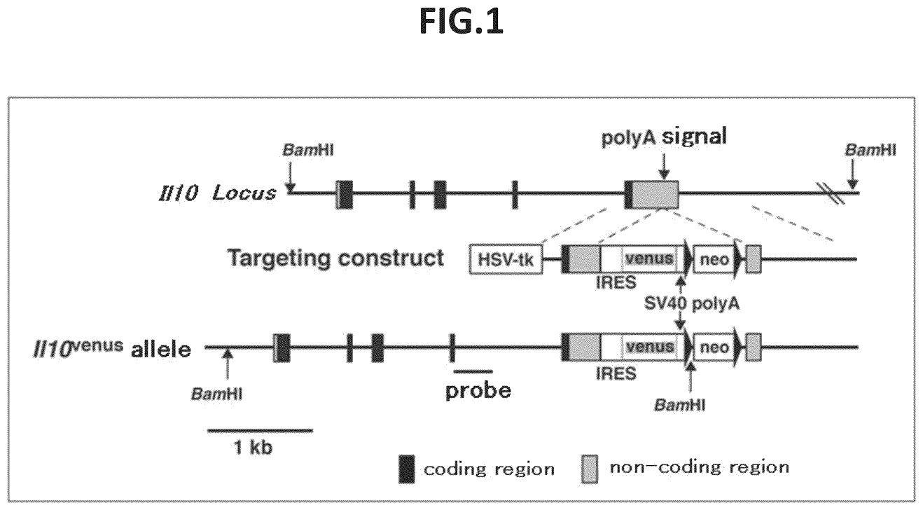

To form a bicistronic locus encoding both Il10 and Venus under control of an Il10 promoter, a targeting construct was first created. Specifically, a cassette (IRES-Venus-SV40 polyA signal cassette, refer to Non-Patent Document 4) which was made of an internal ribosome entry site (IRES), a yellow fluorescent protein (Venus), and a SV40 polyA signal (SV40 polyA) and which was arranged next to a neomycin-resistant gene (neo), was inserted between a stop codon and a polyA signal (Exon 5) of a Il10 gene. Next, the obtained targeting construct was used to cause homologous recombination with the Il10 gene region in the genome of mice. Thus, Il10^{venus} mice having an Il10^{venus} alleles were produced (refer to FIG. 1). Note that in FIG. 1 “tk” represents a gene coding thymidine kinase, “neo” represents the neomycin-resistant gene, and “BamH1” represents a cleavage site by the restriction enzyme BamH1.

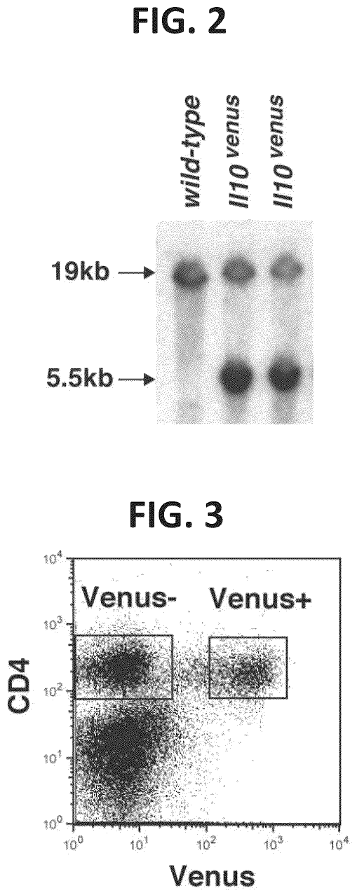

Genomic DNAs were extracted from the Il10^{venus} mice, treated with BamH1, and Southern blotted by use of a probe shown in FIG. 1. FIG. 2 shows the obtained results. Wild-type and Il10^{venus} alleles were detected as bands having sizes of 19 kb and 5.5 kb, respectively. Hence, as is apparent from the results shown in FIG. 2, it was found that the homologous recombination shown in FIG. 1 occurred in the genome of the Il10^{venus} mice.

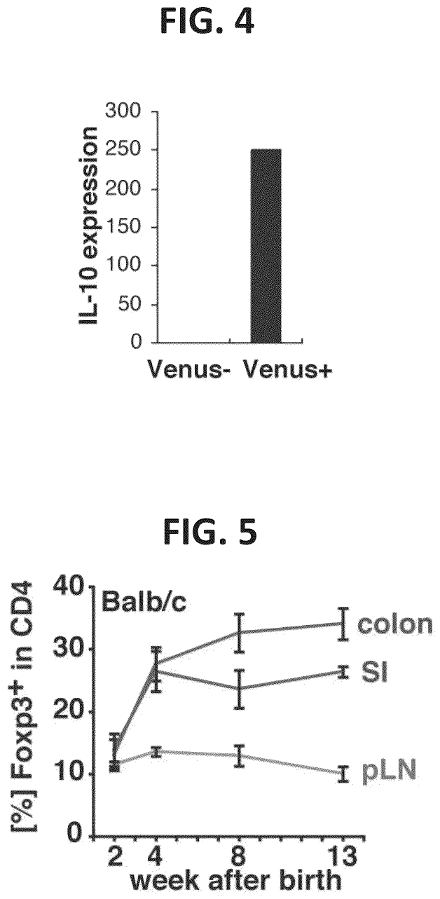

Further, CD4⁺ Venus⁻ cells or CD4⁺ Venus⁺ cells in the colonic lamina propria of the Il10^{venus} mice were sorted by use of a FACSAria. Then, real-time RT-PCR was carried out on an ABI 7300 system by a method to be described later, to determine the amount of IL-10 mRNA expressed. FIGS. 3 and 4 show the obtained results. As is apparent from the results shown in FIGS. 3 and 4, it was found that, since the development of the IL-10 mRNA was detected only in the CD4⁺ Venus⁺ cells, the expression of IL-10 mRNA in the Il10^{venus} mice was correctly reflected in the expression of Venus. Note that the germ-free states of such Il10^{venus} mice were established in Central Institute for Experimental Animals (Kawasaki, Japan). The Il10^{venus} mice in the germ-free states were maintained in vinyl isolators in Sankyo Labo Service Corporation, Inc. (Tokyo, Japan), and used in the following Examples.

Meanwhile, experiments and analyses in Examples were carried out as follows.

### Method for Colonization of Mice with Bacteria and Analysis Thereof

According to the description in Non-Patent Documents 5 and 6, mice in which SFB or Clostridium were colonized were produced. Cecal contents or feces of the obtained gnotobiotic mice were dissolved in sterile water or an anaerobic dilution solution. The dissolved cecal contents or feces as they were or after a chloroform treatment were orally administered to GF mice. Three strains of the Lactobacillus and 16 strains of the Bacteroides were cultured separately from each other in a BL or EG agar medium in an anaerobic manner. The cultured bacteria were harvested, suspended in an anaerobic TS broth, and orally administrated forcibly to GF mice. The state of the colonization of the bacteria in the mice was assessed by microscopic observation conducted on a smear preparation of fecal pellets.

### Cell Separation and Flow Cytometry

In order to isolate lymphocytes from the colonic lamina propria and the small intestinal lamina propria, the small intestine and the colon were collected, and cut open longitudinally. Then, fecal content and the like there inside were washed to remove. Subsequently, the small intestine and the colon were shaken in HBSS containing 5 mM of EDTA at 37° C. for 20 minutes. After removal of epithelium and fat tissue, the intestinal tissues were cut into small pieces. To the small pieces, RPMI 1640 (4% fetal bovine serum (FBS), 1 mg/ml of collagenase D, 0.5 mg/ml of dispase, and 40 μg/ml of DNaseI (all of which were manufactured by Roche Diagnostics K.K.)) were added, and the mixture was shaken in a water bath kept at 37° C. for 1 hour. The digested tissues were washed with HBSS containing 5 mM of EDTA, and resuspended in 5 ml of 40% PERCOLL® (GE Healthcare). The suspension was overlayered on 2.5 ml of 80% PERCOLL® in a 15-ml Falcon tube. Then, centrifugation was carried out at room temperature and at 2000 rpm for 20 minutes to conduct cell separation by PERCOLL® density gradient centrifugation. Cells at the interface were collected, and used as lamina propria lymphocytes. The collected cells were suspend in a staining buffer (PBS, 2% FBS, 2 mM EDTA, and 0.09% NaN₃), and stained by use of an anti-CD4 antibody (RM4-5, BD Biosciences) labeled with PE or PE-Cy7. After the staining of CD4, Foxp3 in the cells were stained by use of CYTOFIX/CYTOPERM® Kit Plus with GOLGISTOP® (BD Biosciences) or Foxp3 Staining Buffer Set (eBioscience), as well as an anti-Foxp3 antibody (FJK-16s, eBioscience) labeled with Alexa647. Flow cytometry was performed by use of a FACScant II, and the data were analyzed by FLOWJO® software (TreeStar Inc.). The sorting of the cells were performed by use of a FACSARIA™.

### Real-Time RT-PCR

From an RNA prepared by using RNeasy Mini Kit (Qiagen), a cDNA was synthesized by use of a MMV reverse transcriptase (Promega KK). The obtained cDNA was analyzed by real-time RT-PCR using Power SYBR Green PCR Master Mix (Applied Biosystems) and ABI 7300 real time PCR system (Applied Biosystems), or real-time RT-PCR using SYBR Premix Ex Taq (TAKARA) and Light Cycler 480. For each sample, a value obtained was normalized for the amount of GAPDH. A primer set was designed by using Primer Express Version 3.0 (Applied Biosystems), and those exhibiting a 90% or higher sequence identity at an initial evaluation were selected. The primer set used was as follows:

Foxp3
(SEQ ID NO: 1)
5′-GGCAATAGTTCCTTCCCAGAGTT-3′
(SEQ ID NO: 2)
5′-GGGTCGCATATTGTGGTACTTG-3′
CTLA4
(SEQ ID NO: 3)
5′-CCTTTTGTAGCCCTGCTCACTCT-3′
(SEQ ID NO: 4)
5′-GGGTCACCTGTATGGCTTCAG-3′
GITR
(SEQ ID NO: 5)
5′-TCAGTGCAAGATCTGCAAGCA-3′
(SEQ ID NO: 6)
5′-ACACCGGAAGCCAAACACA-3′
IL-10
(SEQ ID NO: 7)
5′-GATTTTAATAAGCTCCAAGACCAAGGT-3′
(SEQ ID NO: 8)
5′-CTTCTATGCAGTTGATGAAGATGTCAA-3′
GAPDH
(SEQ ID NO: 9)
5′-CCTCGTCCCGTAGACAAAATG-3′
(SEQ ID NO: 10)
5′-TCTCCACTTTGCCACTGCAA-3′
Mmp2
(SEQ ID NO: 11)
5′-GGACATTGTCTTTGATGGCA-3′
(SEQ ID NO: 12)
5′-CTTGTCACGTGGTGTCACTG-3′
Mmp9
(SEQ ID NO: 13)
5′-TCTCTGGACGTCAAATGTGG-3′
(SEQ ID NO: 14)
5′-GCTGAACAGCAGAGCCTTC-3′
Mmp13
(SEQ ID NO: 15)
5′-AGGTCTGGATCACTCCAAGG-3′
(SEQ ID NO: 16)
5′-TCGCCTGGACCATAAAGAA-3′
Ido1
(SEQ ID NO: 17)
5′-AGAGGATGCGTGACTTTGTG-3′
(SEQ ID NO: 18)
5′-ATACAGCAGACCTTCTGGCA-3′.

### Preparation and Culturing of Large Intestinal Epithelial Cells (IECs)

First, the colon was collected, cut open longitudinally, and rinsed with PBS. Subsequently, the colon was treated with 1 mM dithiothreitol (DTT) at 37° C. for 30 minutes on a shaker, and then vortexed for one minute to disrupt the epithelial integrity. The released IECs were collected, and suspended in 5 ml of 20% PERCOLL®. The suspension was overlayered on 2.5 ml of 80% PERCOLL® in a 15-ml Falcon tube. Then, the tube was centrifuged at 25° C. and 780 g for 20 minutes to conduct cell separation by PERCOLL® density gradient centrifugation. Cells at the interface were collected, and used as colonic IECs (purity: 90% or higher, viability: 95%). The obtained IECs thus collected were suspended in RPMI containing 10% FBS, and 1×10⁵ cells of the IECs were cultured in a 24-well plate for 24 hours. Thereafter, the culture supernatant was collected, and measured for active TGF-β1 level by ELISA (Promega).

Meanwhile, for culturing T cells in vitro, 1.5×10⁵ MACS-purified splenic CD4⁺ T cells were cultured in each well of a round-bottomed 96-well plate, together with a 50% conditioned medium in which IECs isolated from GF mice or Clostridium-colonized mice were cultured, and with 25 ng/ml of hIL-2 (Peprotech), in the presence or absence of 25 μg/ml of an anti-TGF-β antibody (R&D). Note that 10 μg/ml of an anti-CD3 antibody and an anti-CD28 antibody (BD Bioscience) were bound to the round-bottomed plate. After a 5-day culture, the CD4⁺ T cells were collected, and subjected to a real-time PCR.

### Colitis Experimental Model

A fecal suspension of Clostridium-colonized mice was orally administered to C57BL/6 mice (2-week old), and grown in a conventional environment for six weeks.

For preparing a DSS-induced colitis model, 2% (wt/vol) DSS (reagent grade, DSS salt, molecular weight=36 to 50 kD, manufactured by MP Biomedicals), together with drinking water, was given to the mice for six days.

Meanwhile, for preparing an oxazolone-induced colitis model, the mice were presensitized by transdermally applying, onto the mice, 150 μl of a 3% oxazolone (4-ethoxymethylene-2-phenyl-2-oxazolin-5-one, Sigma-Aldrich)/100% ethanol solution. Five days after that, 150 μl of a 1% oxazolone/50% ethanol solution was intrarectally administered again to the presensitized mice under a light anesthesia. Note that the intrarectal administration was conducted by using a 3.5F catheter.

Each mouse was analyzed daily for body weight, occult blood, bleeding visible with the naked eyes (gross blood), and the hardness of stool. Moreover, the body weight loss percentage, intestinal bleeding (no bleeding, occult blood (hemoccult+), or bleeding visible with the naked eyes), and the hardness of stool (normal stool, loose stool, or diarrhea) were evaluated numerically, and the disease activity index (DAI) was calculated in accordance with the description in “S. Wirtz, C. Neufert, B. Weigmann, M. F. Neurath, Nat Protoc 2, 541 (2007).”

### OVA Specific IgE Reaction

BALB/c SPF mice were inoculated with a fecal suspension of Clostridium-colonized mice (2-week old), and grown in a conventional environment. Then, 1 μg of OVA (grade V, Sigma) and 2 mg of alum (Thermo Scientific), 0.2 ml in total, were intraperitoneally injected to the mice (at their ages of 4 weeks and 6 weeks). Sera were collected every week from the mice at the root of their tail, and OVA-specific IgE was measured by ELISA (Chondrex). Then, at their ages of 8 weeks, splenic cells were collected, inoculated in a 96-well plate at 1×10⁶ cells per well, and stimulated with OVA (100 μg/ml) for three days. Thereafter, the culture supernatant was collected, and measured for IL-4 and IL-10 levels by ELISA (R&D).

### Statistical Analysis

The difference between control and experimental groups was evaluated by the Student's t-test.

### Example 1

First, it was investigated whether or not accumulation of regulatory T cells (Treg cells) in the colonic lamina propria was dependent on commensal bacteria. Specifically, lymphocytes were isolated from peripheral lymph nodes (pLN) of Balb/c mice bred in the absence of specific pathogenic bacteria (SPF) or from lamina propria of the colon or the small intestine (SI) of the mice. The CD4 and Foxp3 were stained by antibodies. Then, the ratio of Foxp3⁺ cells in CD4⁺ lymphocytes was analyzed by flow cytometry. FIG. 5 shows the obtained results. As is apparent from the results shown in FIG. 5, it was found that Foxp3⁺ Treg cells were present at a high frequency in the lamina propria of the gastrointestinal tracts, especially in the colonic lamina propria, of the mice kept under the environment free from specific pathogenic microorganisms (SPF). In addition, it was also found that the number of the Foxp3⁺ Treg cells in the colonic lamina propria gradually increased up to three months after their birth, whereas the number of the Foxp3⁺ Treg cells in the peripheral lymph nodes was basically constant from the time of two weeks after their birth.

### Example 2

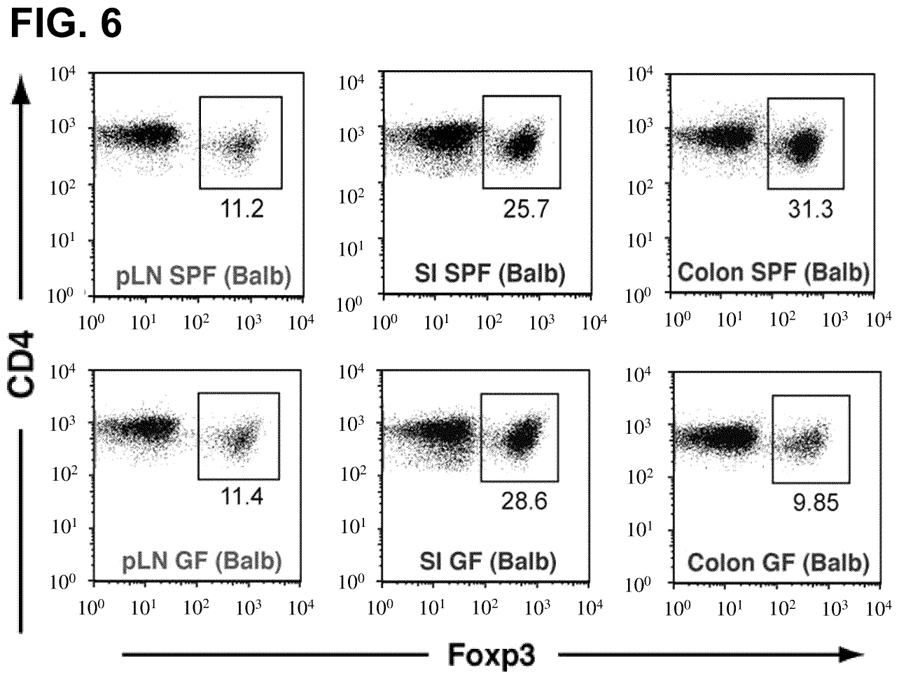

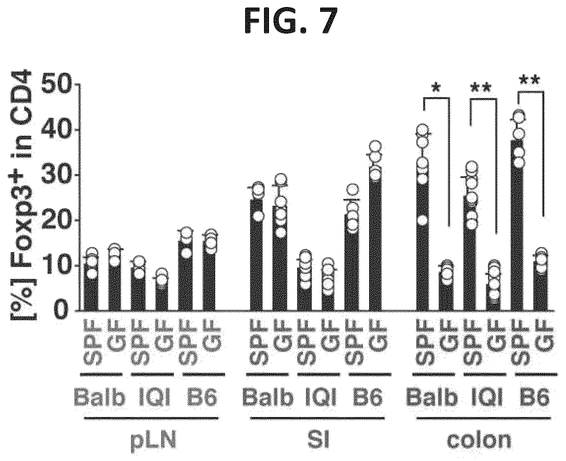

Next, it was investigated whether or not the temporal accumulation of the Treg cells in the colon as found in Example 1 had a relationship with the colonization of intestinal commensal microbiota. Specifically, the expression of CD4 and the expression of Foxp3 in lymphocytes isolated from the small intestine, the colon, and the peripheral lymph nodes of mice bred under a germ-free (GF) or SPF environment (8 weeks old: Balb/c mice, IQI mice, and C57BL/6 mice) were analyzed. Similar results were obtained in three or more independent experiments. FIGS. 6 and 7 show the obtained results. Note that, in FIG. 7, each white circle represents the absolute number of CD4⁺ Foxp3⁺ cells in an individual mouse, and the error bars represent standard deviations (SDs).

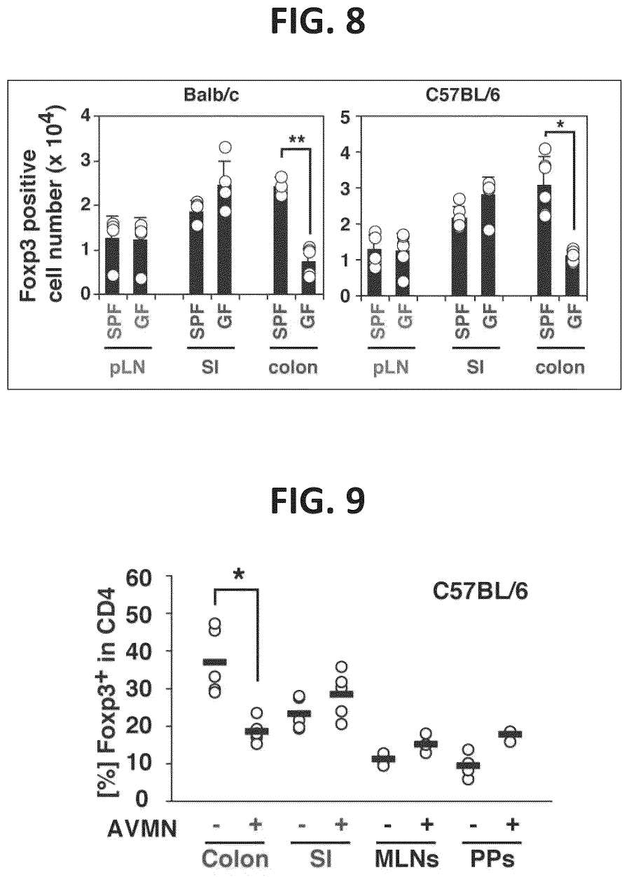

In addition, lamina propria lymphocytes were collected from SPF mice and GF mice (Balb/c mice or C57BL/6 mice). CD4 and Foxp3 were stained with antibodies. Then, the lamina propria lymphocytes were analyzed by FACS. FIG. 8 shows the obtained results. Note that in FIG. 8 each white circle represents the absolute number of CD4⁺ Foxp3⁺ cells in an individual mouse, ** indicates that “P<0.001”, and * indicates that “P<0.01.”

Further, lymphocytes were isolated from the lamina propria of the colon, the lamina propria of the small intestine (SI), Peyer's patches (PPs), and mesenteric lymph nodes (MLNs) of mice (SPF C57BL/6 mice) to which antibiotics were orally administered with water for eight weeks. CD4 and Foxp3 were stained with antibodies. Then, the lymphocytes were analyzed by FACS. Similar results were obtained in two or more independent experiments. FIG. 9 shows the obtained results (the ratio of the Foxp3⁺ cells in the CD4⁺ cells of an individual mouse). Note that the following antibiotics were used in combination in accordance with the description in the following document:
ampicillin (A; 500 mg/L, Sigma)
vancomycin (V; 500 mg/L, NACALAI TESQUE, INC.)
metronidazole (M; 1 g/L, NACALAI TESQUE, INC.)
neomycin (N; 1 g/L, NACALAI TESQUE, INC.)

Rakoff-Nahoum, J. Paglino, F. Eslami-Varzaneh, S. Edberg, R. Medzhitov, Cell 118, 229 (Jul. 23, 2004)

Fagarasan et al., Science 298, 1424 (Nov. 15, 2002)

In FIG. 9, each white circle represents the absolute number of the CD4⁺ Foxp3⁺ cells in an individual mouse, each horizontal bar represents the average value of the absolute numbers, * indicates that “P<0.01,” and “AVMN” represents the kinds of the administered antibiotics by using the first letters of the antibiotics.

As is apparent from the results shown in FIGS. 6 to 9, the frequencies and the absolute numbers of Foxp3⁺ CD4⁺ cells in the small intestine and the peripheral lymph nodes of the GF mice were equal to or greater than those of the SPF mice (refer to FIGS. 6 to 8). In addition, the numbers of the Treg cells in the small intestinal lamina propria, Peyer's patches, and mesenteric lymph nodes of the SPF mice to which the antibiotics were orally administered for eight weeks were equal to or greater than those of the SPF mice (refer to FIG. 9). Meanwhile, the number of the Foxp3⁺ CD4⁺ cells in the colonic lamina propria of the GF mice was decreased significantly in comparison with that of the SPF mice (refer to FIGS. 6 and 7). This decrease was commonly observed among mice of different genetic backgrounds (Balb/c, IQI, and C57BL/6), as well as among mice bred in different animal facilities (refer to FIG. 7 for the data regarding the different genetic backgrounds, the data regarding the mice bred in the different animal facilities are not shown in the drawings). In addition, it was also shown that the number of Treg cells in the colonic lamina propria of the SPF C57BL/6 mice to which the antibiotics were administered was decreased significantly (refer to FIG. 9).

### Example 3

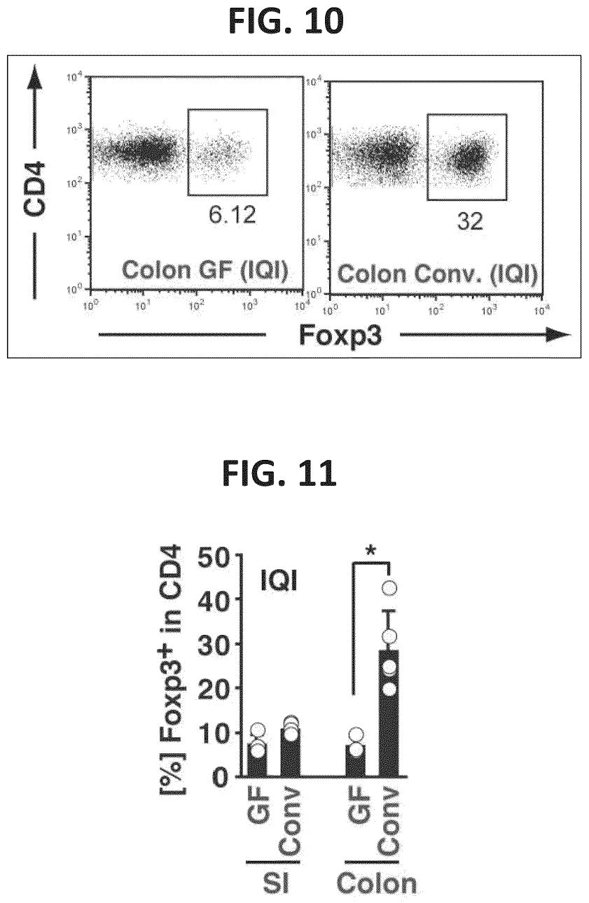

Next, it was directly checked whether or not the decrease in the number of the Treg cells in the colonic lamina propria of the GF mice shown in Example 2 was attributed to the absence of microbiota. Specifically, a fecal suspension of B6 SPF mice purchased from The Jackson Laboratory was orally administered to GF-IQI mice (conventionalization). Three weeks after the administration, lymphocytes were isolated from the colonic lamina propria, and the expression of Foxp3 in CD4⁺ lymphocytes was analyzed. FIGS. 10 and 11 show the obtained results. Note that each white circle in FIG. 11 represents the absolute number of CD4⁺ Foxp3⁺ cells in an individual mouse, the error bars represent standard deviations (SD), * indicates that “P<0.01” in Student's t-test, and ** indicates that “P<0.001.” As is apparent from the results shown in FIGS. 10 and 11, the number of Treg cells in the small intestinal lamina propria did not change. However, the number of the Treg cells in the colonic lamina propria increased significantly. Hence, it was shown that host-microbial interaction played an important role in the accumulation of Foxp3⁺ Treg cells in the colonic lamina propria, while the accumulation of the Treg cells in the small intestinal lamina propria had a different mechanism.

### Example 4

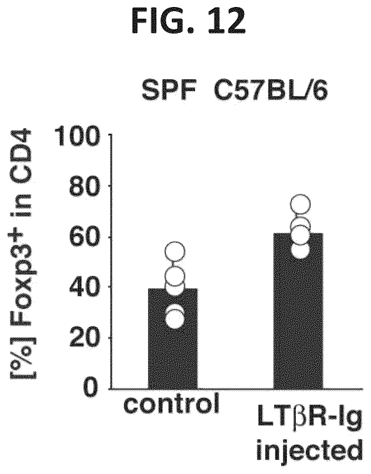

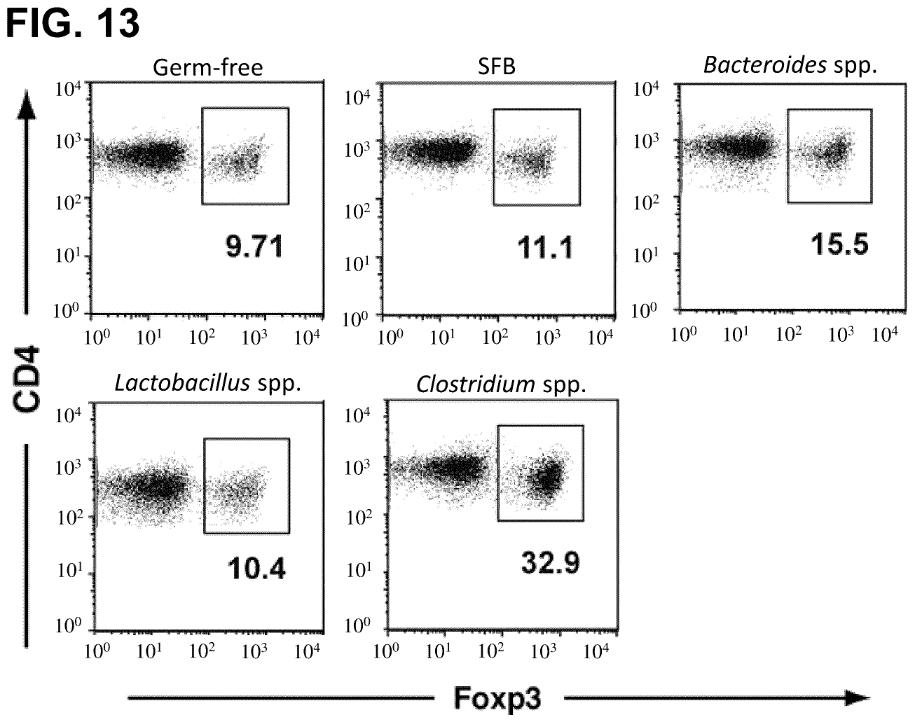

Next, the relationship between the gut-associated lymphoid tissues of mice and the number of Foxp3⁺ cells in the colonic lamina propria of the mice was investigated in accordance with the method described in M. N. Kweon et al., J Immunol 174, 4365 (Apr. 1, 2005). Specifically, 100 μg of an extracellular domain recombinant protein (a fusion protein (LTβR-Ig) between a lymphotoxin β receptor (LTBR) and a Fc region of human IgG1, refer to Honda et al., J Exp Med 193, 621 (Mar. 5, 2001)) was injected intraperitoneally into pregnant C57BL/6 mice 14 days after conception. The LTβR-Ig was again injected intraperitoneally into fetuses obtained from such mice, so that mice from which isolated lymphoid follicles (ILFs), Peyer's patches (PPs), and colonic-patches (CPs) were completely removed were produced. Then, the ratios of Foxp3⁺ cells in CD4⁺ cells in the colonic lamina propria of the mice treated with the LTβR-Ig, and mice treated with rat IgG (control) were analyzed by FACS. FIG. 12 shows the obtained results. Note that in FIG. 12 each white circle represents the ratio of Foxp3⁺ cells in an individual mouse, and the error bars represent standard deviations. As is apparent from the results shown in FIG. 12, it was found that the ratio of the Foxp3⁺ cells in the colonic lamina propria of the mice deficient in isolated lymphoid follicles, Peyer's patches, and the colonic-patches (the mice treated with the LTβR-Ig) rather increased. Accordingly, it was suggested that the decrease in the number of the Treg cells in the colonic lamina propria of the GF mice and the mice treated with the antibiotics was caused because the transmission of specific signals which promotes the accumulation of Treg cells in the colonic lamina propria and which is caused by the intestinal microbes did not occur, rather than simply because of a secondary effect of disorganized gut-associated lymphoid tissues.

### Example 5

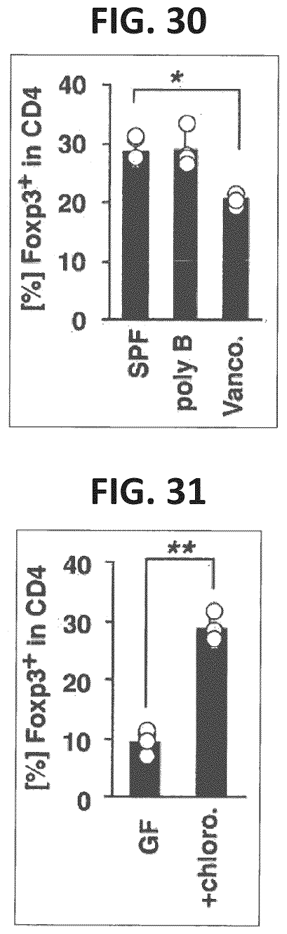

To investigate whether or not a specific intestinal flora induced the accumulation of colonic Treg cells, vancomycin as an antibiotic against Gram-positive bacteria or polymyxin B as an antibiotic against Gram-negative bacteria was administered to SPF mice (from 4 weeks of age) for four weeks, and analyzed for the ratio of Foxp3⁺ cells in the CD4⁺ cell group ([%] Foxp3⁺ in CD4). FIG. 30 shows the obtained results. Note that, in FIG. 30, “SPF” indicates the result of SPF mice (control), “poly B” indicates the result of the SPF mice to which polymyxin B was administered, and “Vanco.” indicates the result of the SPF mice to which vancomycin was administered. Meanwhile, * indicates that “P<0.01.”

As is apparent from the results shown in FIG. 30, the number of Treg cells in the colon of the mice to which vancomycin was administered was markedly decreased in comparison with that of the control. In contrast, no influence was observed on the number of Treg cells of the mice to which polymyxin B was administered. Those facts suggested that Gram-positive commensal bacteria played a major role in accumulation of Treg cells.

### Example 6

A recent report has suggested that spore-forming bacteria play an important role in intestinal T cells response (see V. Gaboriau-Routhiau et al., Immunity 31, 677 (Oct. 16, 2009)). In this respect, fecal microorganisms (spore-forming fraction) resistant to 3% chloroform were orally administered to GF mice, which were then analyzed for the ratio of Foxp3⁺ cells in the CD4⁺ cell group ([%] Foxp3⁺ in CD4). FIG. 31 shows the obtained results. Note that, in FIG. 31, “GF” indicates the result of GF mice, and “+chloro” indicates the result of the GF mice to which the chloroform-treated feces were administered. Meanwhile, ** indicates that “P<0.001.”

As is apparent from the results shown in FIG. 31, three weeks after the administration of the chloroform-treated feces, the number of Treg cells in the administered mice was markedly increased to the same level as those of the SPF mice and the GF mice to which the untreated feces was forcibly administered (see FIGS. 7 and 11).

Accordingly, considering the results shown in Example 5 in combination, it was revealed that the specific components of the indigenous microbiota were highly likely to belong to the Gram-positive group, and that the spore-forming fraction played an important role in the induction of Treg cells.

### Example 7

Next, the species of the intestinal microbiota which induced the accumulation of Treg cells in the colon as suggested in Examples 4 to 6 were identified. Specifically, segmented filamentous bacteria (SFB), 16 strains of the Bacteroides spp. (Bactero. (6 strains of B. vulgatus, 7 of the B. acidifaciens group 1, and 3 of the B. acidifaciens group 2)), 3 strains of the Lactobacillus (Lacto. (L. acidophilus, L. fermentum, and L. murinum)), and 46 strains of Clostridium spp. (Clost., refer to “Itoh, K., and Mitsuoka, T. Characterization of clostridia isolated from faeces of limited flora mice and their effect on caecal size when associated with germ-free mice. Lab. Animals 19: 111-118 (1985))”), or microbiota collected from mice (SPF) bred under a conventional environment was orally administered to GF-Balb/c mice or GF-IQI mice. The mice were maintained in vinyl isolators for three weeks. Then, CD4 cells were isolated from the colon and the small intestine of these mice. The numbers of Treg cells in the colon and the small intestine were analyzed by flow cytometry.

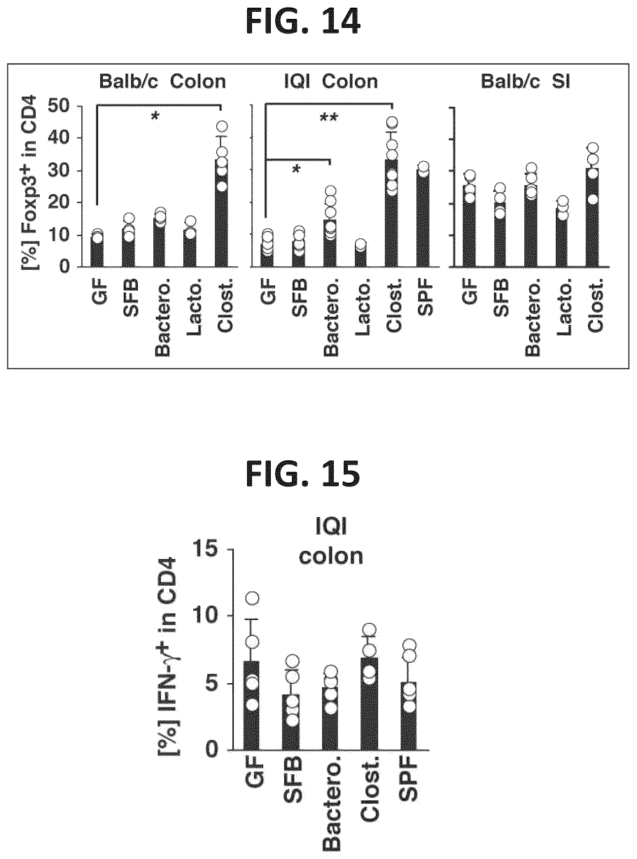

FIG. 13 shows FACS dot-plots obtained when a gate was set on CD4⁺ cells of the Balb/c mice. FIG. 14 shows the ratio of Foxp3⁺ cells in CD4⁺ cells of each mouse.

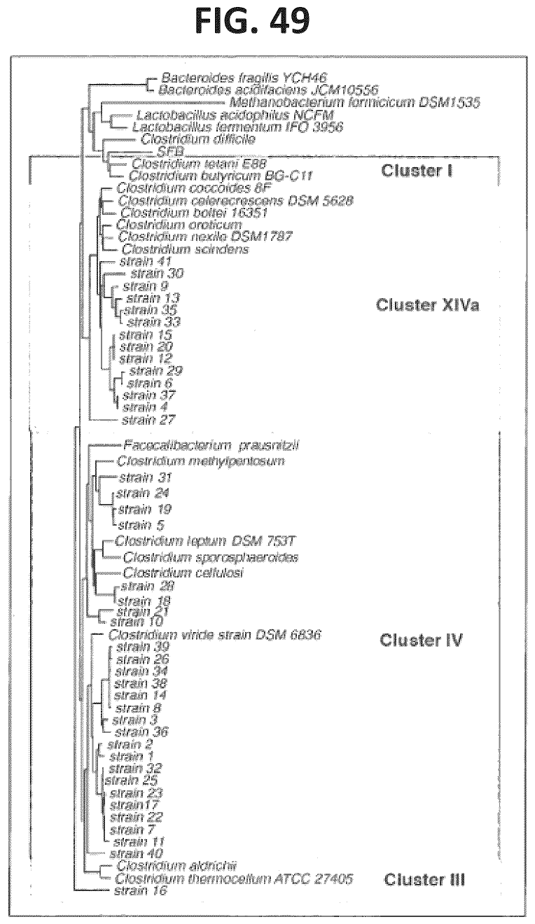

Note that, the bacteria belonging to the genus Clostridium are classified by sequencing of 16S rRNA gene, as follows. Specifically, the 16S rRNA genes of the bacteria were amplified by PCR using 16S rRNA gene-specific primer pairs: 5′-AGAGTTTGATCMTGGCTCAG-3′ (SEQ ID NO: 19) and 5′-ATTACCGCGGCKGCTG-3′ (SEQ ID NO: 20)(see T. Aebischer et al., Vaccination prevents Helicobacter pylori-induced alterations of the gastric flora in mice. FEMS Immunol. Med. Microbiol. 46,221-229(2006)). The 1.5-kb PCR product was then introduced into PCR-Blunt Vector. The inserts were sequenced and aligned using the ClustalW software program. The resulting sequences of 16S rRNA genes derived from strain 1-41 of 46 strains of Clostridium spp. were shown in SEQ ID NO: 21-61. Phylogenetic tree which was constructed by the neighbor-joining method with the resulting sequences of the 41 strains of Clostridium and those of known bacteria obtained from Genbank database using Mega software was shown in FIG. 49.

As is apparent from the results shown in FIGS. 13 and 14, no effect on the number of the Treg cells in the colon was observed in the GF mice in which the segmented filamentous bacteria (SFB) were colonized (refer to FIG. 14). Moreover, mice in which the cocktail of three strains of Lactobacillus was colonized gave similar results (refer to FIG. 14). On the other hand, it was shown that the accumulation of Foxp3⁺ cells in the colonic lamina propria was strongly induced in the mice in which 46 strains of Clostridium spp. were colonized. Importantly, such accumulation was promoted irrespective of the genetic backgrounds of the mice, and led to the increase in number similar to that in the SPF mice although intestinal microbiota of only a single genus were colonized. It was also shown that the colonization of the Clostridium did not change the number of Treg cells in the small intestinal lamina propria (refer to FIG. 14). Note that, when the 16 strains of Bactericides spp. were colonized, the number of Treg cells in the colon was increased significantly. However, the extent of the increase varied depending on the genetic background of the mice in which the bacteria were colonized (refer to FIGS. 13 and 14).

### Example 8

Next, CD4 expression, Foxp3 expression, and Helios expression in LP lymphocytes of the thymuses and the colons of SPF mice, GF mice, Lactobacillus-colonized mice, and Clostridium-colonized mice were analyzed by flow cytometry.

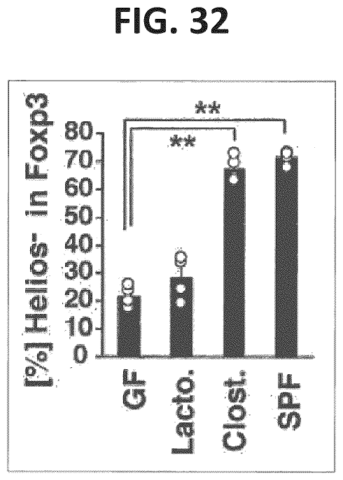

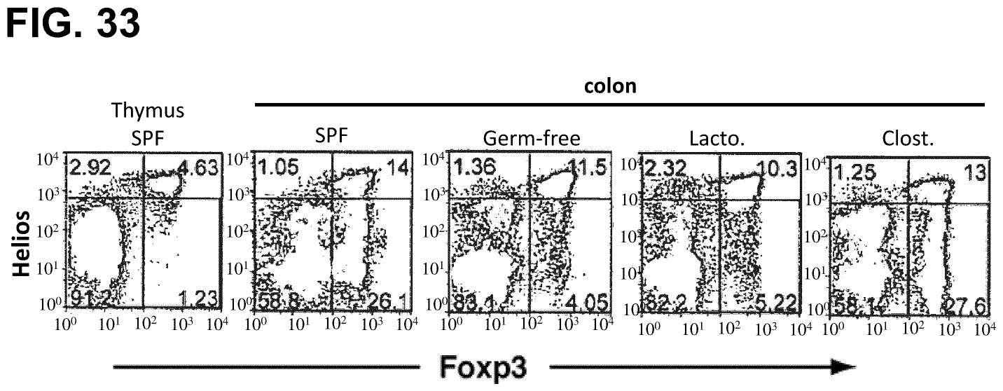

FIGS. 32 and 33 show the obtained results. Note that, in FIGS. 32 and 33, “GF” or “Germ Free” indicates the results of the GF mice, “SPF” indicates the results of the SPF mice, “Lacto.” indicates the results of the Lactobacillus-colonized mice, and “Clost.” indicates the results of the Clostridium-colonized mice. In FIG. 32, the vertical axis represents the ratio of Helios⁻ cells in the Foxp3⁺ cell group ([%] Helios⁻ in Foxp3⁺), and ** indicates that “P<0.001.”

As is apparent from the results shown in FIGS. 32 and 33, most Foxp3⁺ cells found in the SPF mice or the Clostridium-colonized mice did not express Helios. Note that Helios is a transcription factor known to be expressed in thymic-derived natural Treg cells (see A. M. Thornton et al., J Immunol 184, 3433 (Apr. 1, 2010)). Accordingly, it was suggested that most of the Treg cells in the SPF mice and the Clostridium-colonized mice were Treg cells induced in peripheral portions, i.e., so-called iTreg cells.

### Example 9

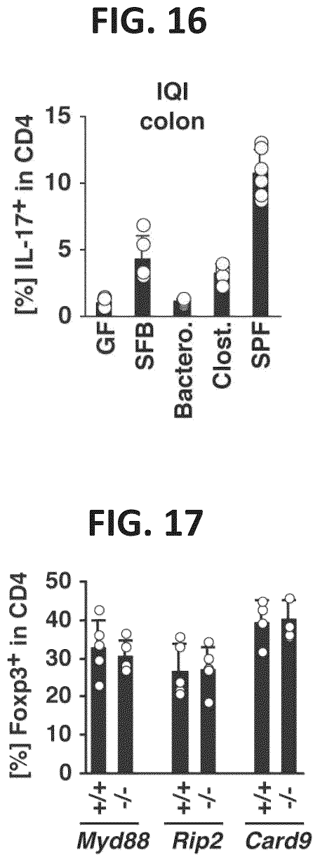

Next, it was investigated whether or not the colonization of the Clostridium or the like had an influence on other T cells. Specifically, SFB, 16 strains of Bacteroides spp. (Bactero.), 46 strains of Clostridium spp. (Clost.), or microbiota collected from mice bred under a conventional environment (SPF) was colonized in GF IQI mice. Three weeks later, lymphocytes in the colonic lamina propria were isolated from these mice, and stimulated with PMA (50 ng/ml) and ionomycin (1 μg/ml) for four hours in the presence of GOLGISTOP® (BD Bioscience). After the stimulation was given, intracellular cytokines were stained by using an anti-IL-17 PE antibody (TC11-18H10) and an anti-IFN-g FITC antibody (BD Bioscience) in accordance with the manual of a CYTOFIX/CYTOPERM® kit (BD Bioscience). Then, the ratio of IFN-γ⁺ cells or IL-17⁺ cells in CD4⁺ leucocytes was analyzed by flow cytometry. FIGS. 15 and 16 show the obtained results. Note that, in FIGS. 15 and 16, each white circle represents the absolute number of CD4⁺ IFN-γ⁺ cells or the absolute number of CD4⁺ IL-17⁺ cells in each individual mouse, and the error bars represent standard deviations (SD). As is apparent from the results shown in FIGS. 15 and 16, the colonization of the Clostridium did not have any influence on Th1 cells (CD4⁺ IFN-γ⁺ cells) in the colon, and caused only a slight increase of Th17 cells (CD4⁺ IL-17⁺ cells). Accordingly, it was suggested that the genus Clostridium was a genus of bacteria which specifically induced Treg cells.

### Example 10

It has been reported that 46 strains of Clostridium spp. exert an influence on the accumulation of CD8⁺ intestinal tract intraepithelial lymphocytes (IELs) in the colon. Accordingly, it is conceivable that Clostridium regulates the immune system in various aspects, and that Clostridium exhibits a marked ability to induce and maintain Treg cells especially in the colon, as described above. In addition, a kind of cytokines, transforming growth factor-β (TGF-β), is known to play an important role in regulation of Treg cell generation.

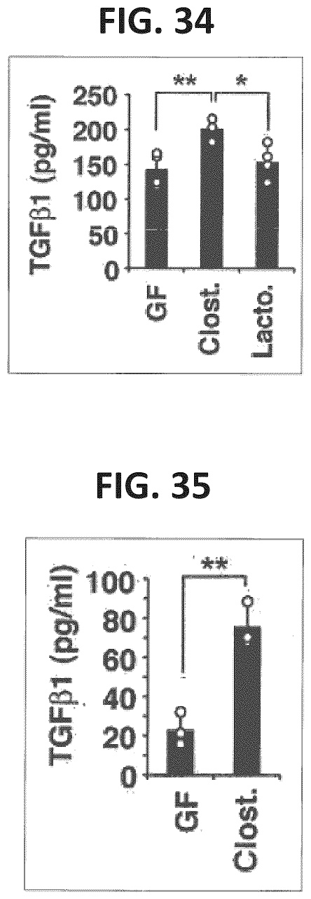

In this respect, it was examined whether or not the colonization of Clostridium provided a colonic environment rich in TGF-β. Specifically, first, the whole colons of GF mice, Clostridium-colonized mice, and Lactobacillus-colonized mice were cultured for 24 hours, and the culture supernatants thereof were measured for the concentration of active TGF-β (TGF-β1) by ELISA (the number of mice analyzed was four per group). FIG. 34 shows the obtained results. Note that, in FIG. 34, “GF” indicates the result of the GF mice, “Clost.” indicates the result of the Clostridium-colonized mice, and “Lacto.” indicates the result of Lactobacillus-colonized mice. Meanwhile, * indicates that “P<0.02,” and ** indicates that “P<0.001.”

As is apparent from the results shown in FIG. 34, the amount of TGF-β produced in the colons of the Clostridium-colonized mice was significantly larger than those of the GF mice and the Lactobacillus-colonized mice.

Next, intestinal epithelial cells (IECs) of GF mice and Clostridium-colonized mice were cultured for 24 hours, and the culture supernatants thereof were measured for the concentration of active TGF-β (TGF-β1) by ELISA (the number of mice analyzed was four per group). FIG. 35 shows the obtained results. Note that, in FIG. 35, “GF” indicates the result of the GF mice, and “Clost.” indicates the result of the Clostridium-colonized mice. Meanwhile, ** indicates that “P<0.001.”

As is apparent from the results shown in FIG. 35, TGF-β was detected in the culture supernatant of the IECs isolated from the Clostridium-colonized mice, whereas no TGF-β was detected in the culture supernatant of the IECs isolated from the GF mice.

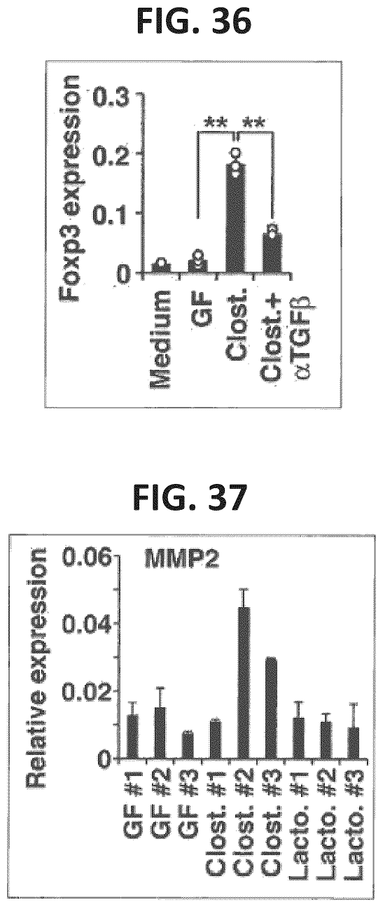

Next, as described above, splenic CD4⁺ T cells were cultured for five days together with a 50% conditioned medium in which IECs isolated from the GF mice or the Clostridium-colonized mice were cultured, and with the anti-CD3 antibody, in the presence or absence of an anti-TGF-β antibody. Then, the T cells were collected, and analyzed for expression of Foxp3 by real-time RT-PCR. FIG. 36 shows the obtained results. Note that, in FIG. 36, “Medium” indicates the result of a medium in which no cells were cultured, “GF” indicates the result of the conditioned medium in which the IECs of the GF mice were cultured, “Clost.” indicates the result of the conditioned medium in which the IECs of the Clostridium-colonized mice were cultured, and “Clost. +αTGFβ” indicates the result of the conditioned medium to which the anti-TGF-β antibody was added and in which the IECs of the Clostridium-colonized mice were cultured. Meanwhile, ** indicates that “P<0.001.”

As is apparent from the results shown in FIG. 36, when the culture supernatant of the IECs derived from the Clostridium-colonized mice was added to the splenic CD4⁺ T cells, the differentiation into Foxp3-expressing cells was accelerated. Meanwhile, the differentiation into the Treg cells was inhibited by the anti-TGF-β antibody.

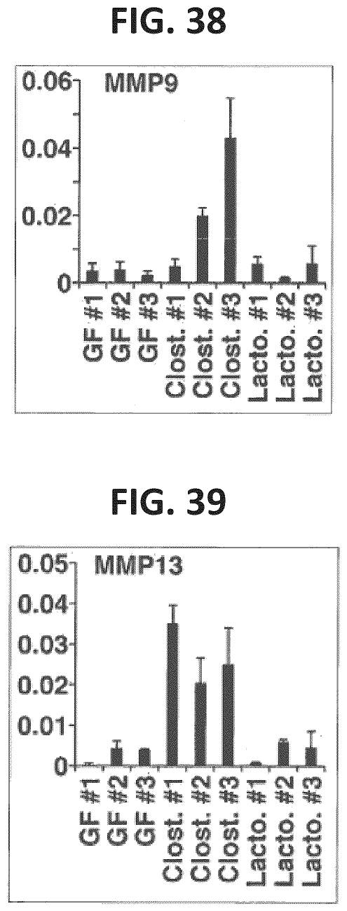

Moreover, the expression of MMP2, MMP9, and MMP13, which are thought to contribute to the activation of latent TGF-β was investigated. The expression of indoleamine 2,3-dioxygenase (IDO), which is thought to be involved in the induction of Treg cells, was also investigated. Specifically, 46 bacterial strains of the genus Clostridium (Clost.), or three bacterial strains of the genus Lactobacillus (Lacto.) were orally administered to C57BL/6 germ-free mice. Three weeks after the administration, IECs were collected, and analyzed for relative mRNA expression levels of MMP2, MMP9, MMP13, and IDO genes by real-time RT-PCR (the number of mice analyzed was three per group). FIGS. 37 to 40 show the obtained results. Note that, in FIGS. 37 to 40, “GF #1 to 3” indicate the results of GF mice, “Clost. #1 to 3” indicate the results of the Clostridium-colonized mice, and “Lacto. #1 to 3” indicate the results of the Lactobacillus-colonized mice.

For the relationship between the activation of latent TGF-β and the above-described MMP, see D'Angelo et al., J. Biol. Chem. 276, 11347-11353, 2001; Heidinger et al., Biol. Chem. 387, 69-78, 2006; Yu et al., Genes Dev. i4, 163-176, 2000. For the relationship between IDO and the induction of Treg cells, see G. Matteoli et al., Gut 59, 595 (May 2010).

As is apparent from the results shown in FIGS. 37 to 39, in agreement with the production of TGF-β described above, transcription products of the genes encoding MMP2, MMP9, and MMP13 were expressed at higher levels in the IECs derived from the Clostridium-colonized mice than those in the GF mice and in the Lactobacillus-colonized mice.

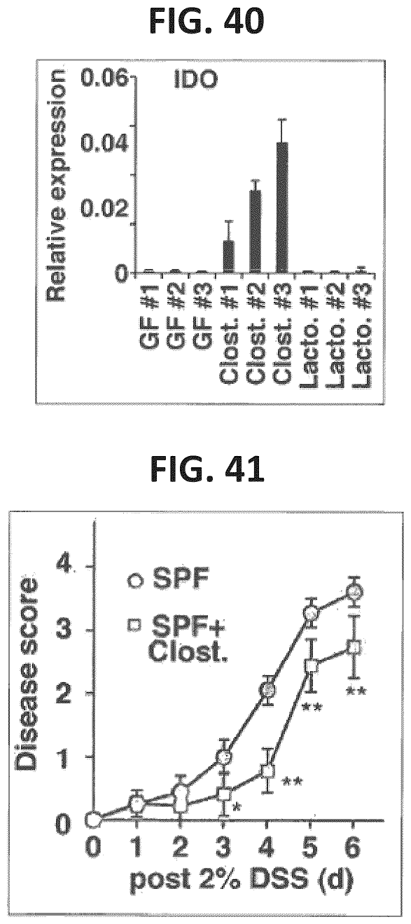

Moreover, as is apparent from the results shown in FIG. 40, IDO was expressed only in the Clostridium-colonized mice.

Accordingly, it was revealed that the Clostridium activated the IECs, and led to the production of TGF-β and other Treg cell-inducing molecules in the colon.

### Example 11

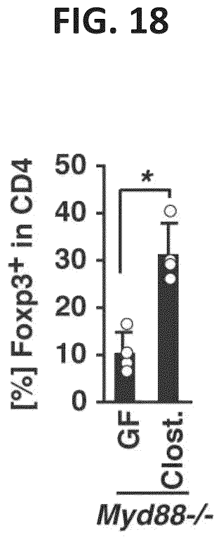

Next, it was investigated whether or not the Treg cell accumulation induced by the colonization of the Clostridium was dependent on signal transmission by pathogen-associated molecular pattern recognition receptors. Specifically, the numbers of Treg cells in the colonic lamina propria of each of SPF mice of Myd88^{-/-} (deficient in Myd88 (signaling adaptor for Toll-like receptor)), Rip2^{-/-} (deficient in Rip2 (NOD receptor adaptor)), and Card9_{-/-} (deficient in Card9 (essential signal transmission factor for Dectin-1 signal transmission)) were examined. In addition, Clostridium spp. were caused to be colonized in the Myd88^{-/-} GF mice, and the change in the number of Treg cells was investigated. FIGS. 17 and 18 show the obtained results. As is apparent from the results shown in FIGS. 17 and 18, the number of Treg cells of each kind of the SPF mice deficient in the associated factors of the pathogen-associated molecular pattern recognition receptors did not change relative to that of wild-type mice of the same litter, which served as a control. In addition, it was found that also when Clostridium spp. were colonized in GF mice deficient in Myd88, the accumulation of Treg cells in the colonic lamina propria was induced. Accordingly, it has been suggested that the mechanism of inducing the accumulation of Treg cells in the colonic lamina propria relies not on activation of recognition pathway for major pathogen-associated molecular patterns as is caused by most of bacterium, but on specific commensal bacterial species.

### Example 12

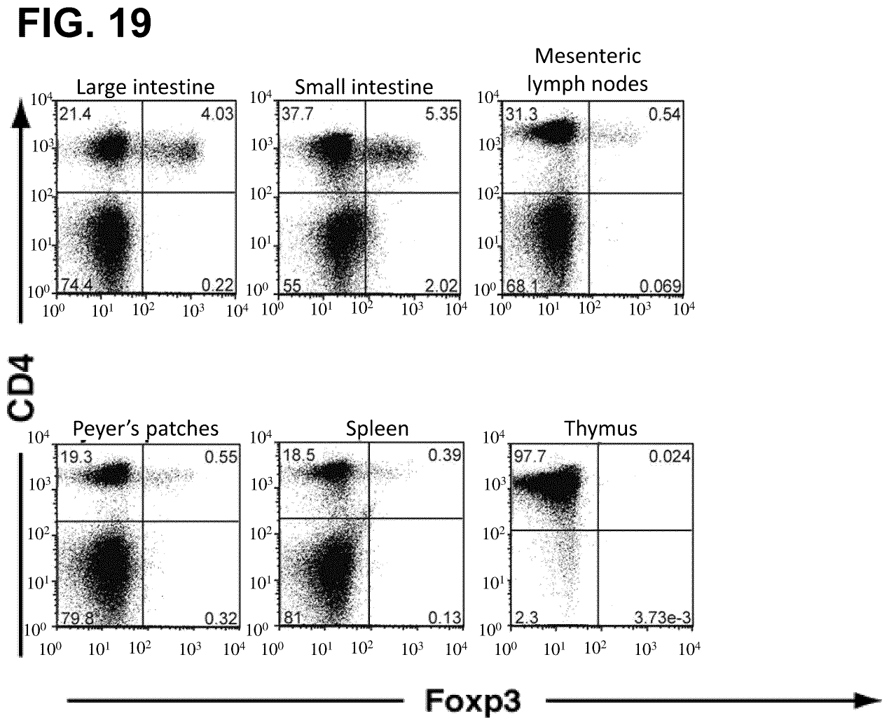

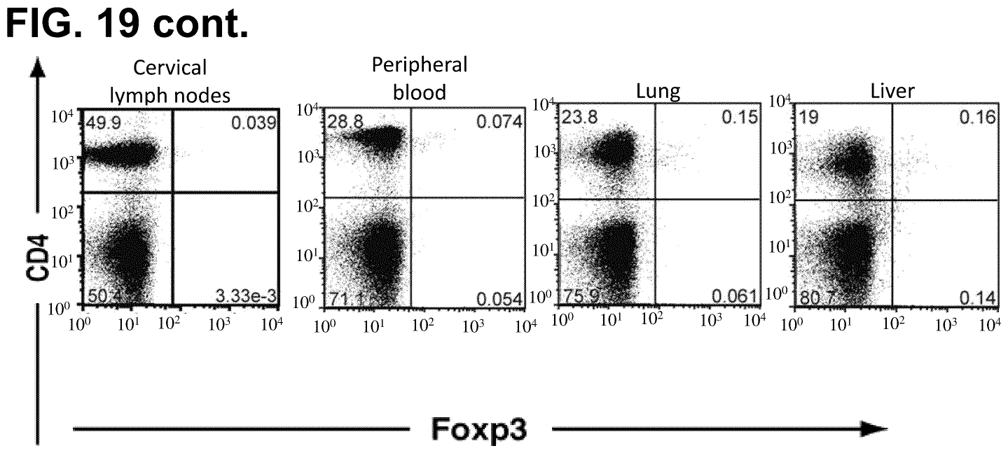

Intestinal tract Foxp3⁺ Treg cells are known to exert some immunosuppressive functions through IL-10 production (refer to Non-Patent Document 9). Meanwhile, animals having CD4⁺ Foxp3⁺ cells from which IL-10 is specifically removed are known to develop inflammatory bowel disease (refer to Non-Patent Document 18). In this respect, first, the expression of IL-10 in lymphocytes of various tissues was examined. Specifically, lymphocytes were isolated from various tissues of SPF Il10^{venus} mice, and the expression of CD4 and the expression of Venus were analyzed by flow cytometry. FIG. 19 shows the obtained results. Note that each numeric value in FIG. 19 represents the ratio of cells within the corresponding one of regions divided into four.

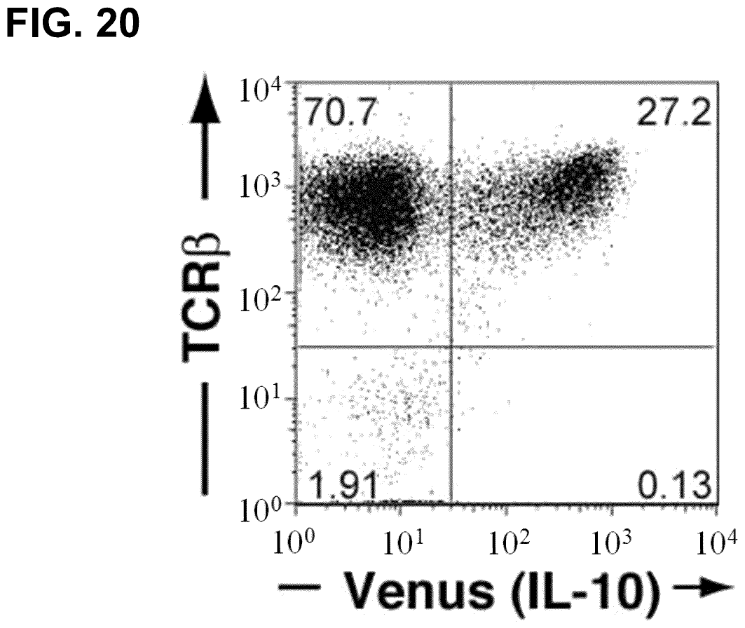

Moreover, lymphocytes in the colonic lamina propria were isolated from Il10^{venus} mice, and the expression of T cell receptor β chain (TCRβ) on the surfaces of the cells was detected by FACS. FIG. 20 shows the obtained results (FACS dot-plots obtained when a gate was set on CD4⁺ cells). Note that each numeric value in FIG. 20 represents the ratio of cells within the corresponding one of regions divided into four.

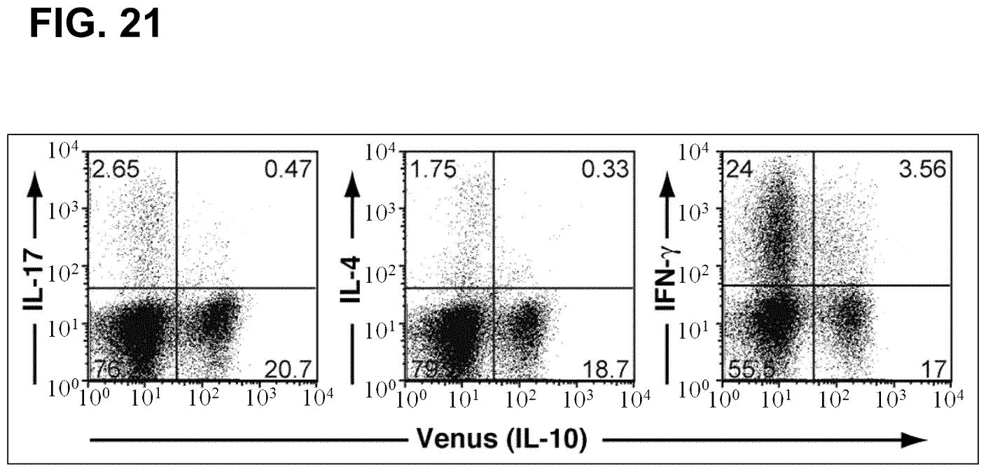

Furthermore, lymphocytes in the colonic lamina propria were isolated from Il10^{venus} mice. The lymphocytes were stimulated with PMA (50 ng/ml) and ionomycin (1 μg/ml) for four hours in the presence of GOLGISTOP® (BD Bioscience). Then, after the stimulation was given, intracellular cytokines were stained by using an anti-IL-17 PE antibody, an anti-IL-4 APC antibody (11B11), and an anti-IFN-g FITC antibody (BD Bioscience) in accordance with the manual of a CYTOFIX/CYTOPERM® kit (BD Bioscience). FIG. 21 shows the obtained results (FACS dot-plots obtained when a gate was set on CD4⁺ cells).

Note that each numeric value in FIG. 21 represents the ratio of cells within the corresponding one of regions divided into four.

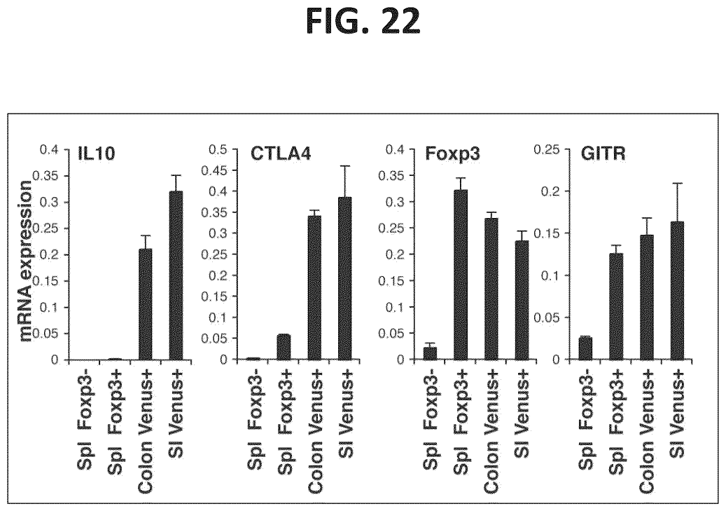

In addition, Foxp3⁺ CD4⁺ cells and Foxp3⁻ CD4⁺ cells were isolated from the spleen (Spl) of Foxp3^{eGFP} reporter mice, and Venus⁺ cells were isolated from the colonic lamina propria and the small intestine (SI) lamina propria of Il10^{venus} mice. Then, the obtained cells were analyzed in terms of the expression of predetermined genes. The gene expression was analyzed by real-time RT-PCR using a Power SYBR Green PCR Master Mix (Applied Biosystems) and an ABI 7300 real time PCR system (Applied Biosystems). Here, the value for each cell was normalized for the amount of GAPDH. FIG. 22 shows the obtained results. Note that in FIG. 22 the error bars represent standard deviations.

As is apparent from the results shown in FIGS. 19 to 22, almost no Venus⁺ cells (IL-10-producing cells) were detected in the cervical lymph nodes (peripheral lymph nodes), thymus, peripheral blood, lung, and liver of mice kept under the SPF conditions. Meanwhile, in the spleen, Peyer's patches, and mesenteric lymph nodes thereof, Venus⁺ cells were slightly detected (refer to FIG. 19). On the other hand, many Venus⁺ cells were found in the lymphocytes in the small intestine lamina propria and colonic lamina propria. In addition, most of the Venus⁺ cells in the intestines were positive for CD4, and also positive for T cell receptor β chain (TCRβ) (refer to FIGS. 19 and 20). Moreover, it was found that the Venus⁺ CD4⁺ T cells expressed Foxp3 and other Treg cell-associated factors such as a cytotoxic T-Lymphocyte antigen (CTLA-4) and a glucocorticoid-induced TNFR-associated protein (GITR) although the Venus⁺ CD4⁺ T cells showed none of the phenotypes of Th2 (IL-4-producing) and Th17 (IL-17-producing) (refer to FIGS. 21 and 22). In addition, it was shown that the expression level of CTLA-4 in the intestinal Venus⁺ cells was higher than that in the splenic GFP₊ Treg cells isolated from the Foxp3^{eGFP} reporter mice (refer to FIG. 22).

### Example 13

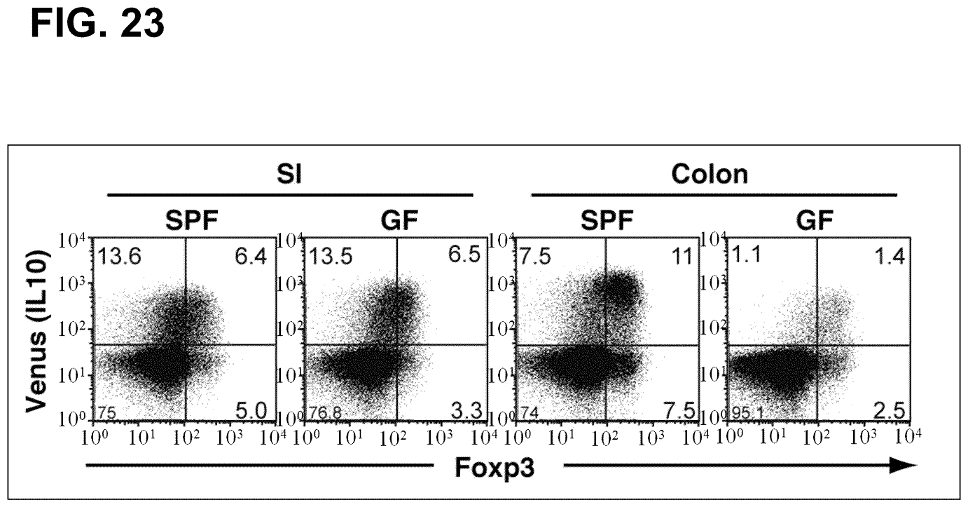

Venus⁺ cells can be classified into at least two subsets, namely, Venus⁺ Foxp3⁺ double positive (DP) Treg cells and Venus⁺ Foxp3⁻ Treg cells on the basis of intracellular Foxp3 expression. Cells of the latter subset correspond to type 1 regulatory T cells (Tr1) (refer to Non-Patent Documents 8 and 9). In this respect, the Venus⁺ cells (IL-10-producing cells) observed in Example 8 were investigated in terms of the expression of Foxp3. Specifically, the expression of CD4, Foxp3, and Venus in the lamina propria of the colon and the lamina propria of the small intestine of Il10^{venus} mice kept under GF or SPF conditions was analyzed by FACS, and the numbers of Venus⁺ cells in the intestinal tract lamina propria were compared between SPF and GF Il10^{Venus} mice. FIG. 23 shows the obtained results (dot-plots obtained when a gate was set on CD4⁺ cells).

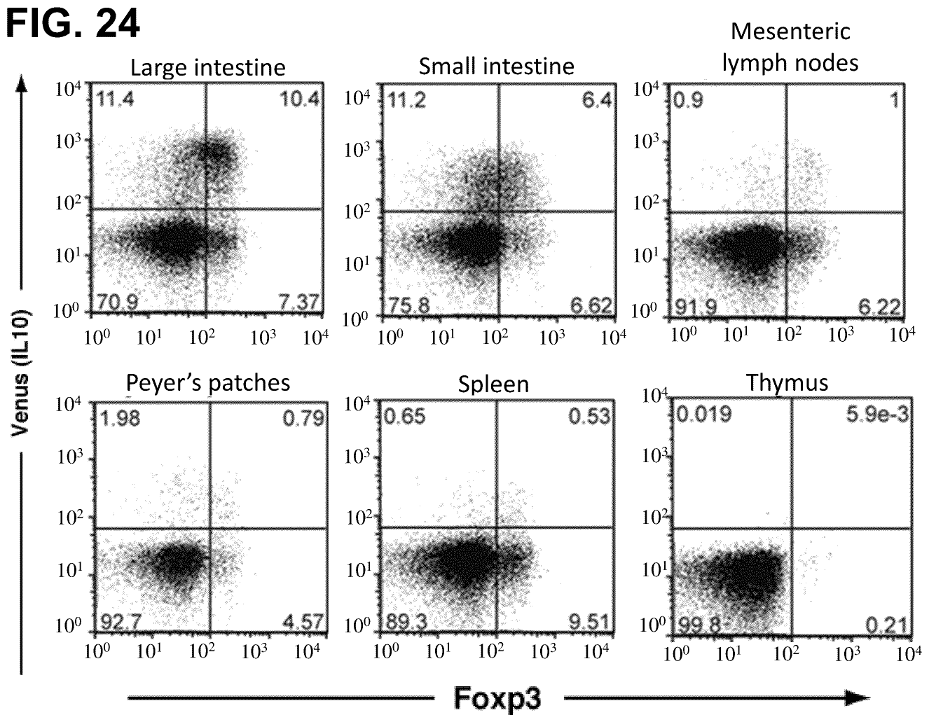

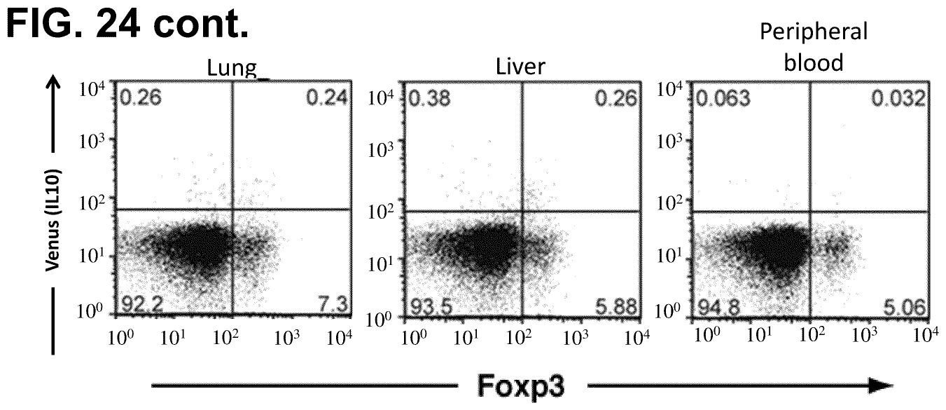

In addition, the intracellular expression of Venus and Foxp3 in CD4 cells in various tissues of SPF Il10^{venus} mice was analyzed by flow cytometry. FIG. 24 shows the obtained results (dot-plots obtained when a gate was set on CD4⁺ cells). Note that each numeric value in FIG. 24 represents the ratio of cells within the corresponding one of regions divided into four.

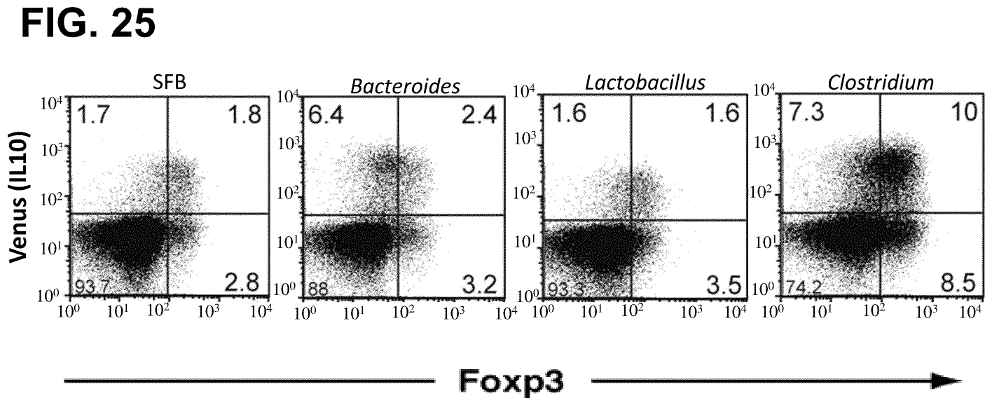

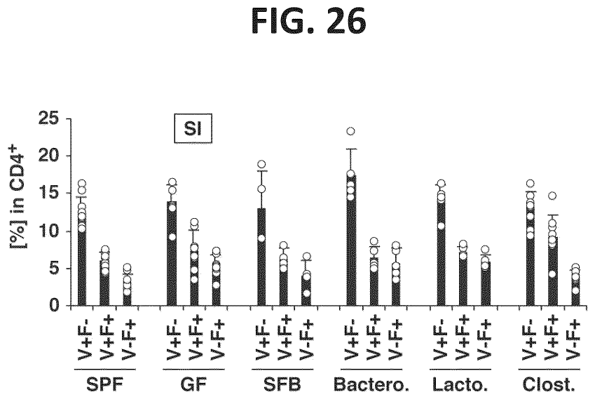

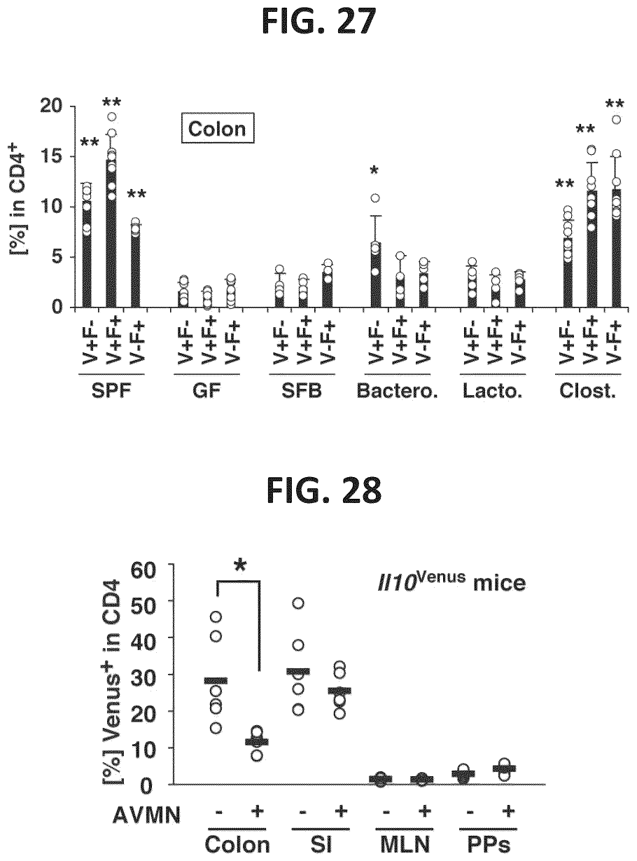

Moreover, in order to investigate whether or not the presence of commensal bacteria had any influence on the expression of IL-10 in regulatory cells in the gastrointestinal tracts, germ-free (GF) Il10^{Venus} mice were prepared. Then, predetermined species of bacteria were caused to be colonized in the obtained GF Il10^{venus} mice. Three weeks after the species of bacteria were colonized, a CD4⁺ cell group (V⁺F⁻, Venus⁺ Foxp3 ⁻cells; V⁺F⁺, Venus⁺ Foxp3⁺ cells; and V⁻F⁺, Venus⁻Foxp3⁺ cells) in which Foxp3 and/or Venus were expressed in the colon and the small intestine was analyzed by flow cytometry. FIG. 25 shows dot-plots obtained when a gate was set on colonic CD4⁺ cells, and FIGS. 26 and 27 show the ratios in the CD4⁺ cell group of each mouse. Note that each numeric value in FIG. 25 represents the ratio of cells within the corresponding one of regions divided into four. Meanwhile, the error bars in FIGS. 26 and 27 represent standard deviations, * indicates that “P<0.02,” and ** indicates that “P<0.001.”

Moreover, in order to check whether or not the presence of commensal bacteria had any influence on the expression of IL-10 in regulatory cells in the gastrointestinal tracts, antibiotics were orally given with water to five or six Il10^{venus} mice per group for 10 weeks. The following antibiotics were used in combination.
ampicillin (A; 500 mg/L Sigma)
vancomycin (V; 500 mg/L NACALAI TESQUE, INC.)
metronidazole (M; 1 g/L NACALAI TESQUE, INC.)
neomycin (N; 1 g/L NACALAI TESQUE, INC.)

Then, CD4 and Foxp3 of lymphocytes in the lamina propria of the colon, the lamina propria of the small intestine (SI), mesenteric lymph nodes (MLN), and Peyer's patches (PPs) were stained with antibodies, and analyzed by FACS. The results were obtained from two or more independent experiments which gave similar results. FIG. 28 shows the obtained results (the ratio of Venus⁺ cells in CD4⁺ cells in each sample). Note that each white circle in FIG. 28 represents an individual sample, each horizontal bar represents an average value, * indicates that “P<0.02,” and “AVMN” represents the kinds of the administered antibiotics by using the first letters of the antibiotics.

As is apparent from the results shown in FIGS. 23 and 24, it was shown that the small intestinal lamina propria was rich in Venus⁺ Foxp3⁻ cells, namely, Tr1-like cells, and that the Venus⁺ Foxp3⁺ DP Treg cells were present at a high frequency in the colon of the SPF mice (refer to FIGS. 23 and 24). In contrast, although sufficient numbers of Foxp3⁺ cells were observed also in other tissues, the expression of Venus was not observed in almost all of the cells (refer to FIG. 24).

In addition, as is apparent from the results shown in FIGS. 23 and 25 to 28, it was shown that all regulatory T cell fractions of Venus⁺ Foxp3⁻, Venus⁺ Foxp3⁺, and Venus⁻ Foxp3⁺ in the colon significantly decreased under the GF conditions (FIGS. 23 and 26 to 27). Moreover, similar decrease in Venus⁺ cells was observed also in the SPF Il10^{Venus} mice treated with the antibiotics (refer to FIG. 28).

Moreover, as is apparent from the results shown in FIGS. 25 to 27, the colonization of Clostridium spp. strongly induced all regulatory T cell fractions of Venus⁺ Foxp3⁻, Venus⁺ Foxp3⁺, and Venus⁻ Foxp3⁺ in the colon, and the degrees of the induction thereof were equal to those in the SPF mice (refer to FIGS. 25 and 27). In addition, it was found that the colonization of the three strains of Lactobacillus or the colonization of SFB had an extremely small influence on the number of Venus⁺ and/or Foxp3⁺ cells in the colon (refer to FIGS. 25 and 27). Moreover, the colonization of 16 strains of Bacteroides spp. also induced Venus⁺ cells, but the influence of the colonization was specific to Venus⁺ Foxp3⁻Tr1-like cells (refer to FIGS. 25 and 27). On the other hand, it was found that none of the bacterial species tested exerted any significant influence on the number of IL-10-producing cells in the small intestinal lamina propria (refer to FIG. 26).

Hence, it was shown that the genus Clostridium colonized in the colon or a physiologically active substance derived from the bacteria provided a signal for inducing the accumulation of IL-10⁺ regulatory T cells in the colonic lamina propria or the expression of IL-10 in T cells. Meanwhile, it was shown that the number of Venus⁺ cells in the small intestine was not significantly influenced by the situation where no commensal bacteria were present or commensal bacteria were decreased (refer to FIGS. 23 and 26 to 28), and that IL-10⁺ regulatory cells (Tr1-like cells) accumulated in the small intestinal lamina propria independently of commensal bacteria.

### Example 14

It was investigated whether or not Venus⁺ cells induced by the genus Clostridium had an immunosuppressive function similar to that of Venus⁺ cells in the colon of SPF mice. Specifically, CD4⁺ CD25⁻ cells (effector T cells, Teff cells) isolated from the spleen were seeded in a flat-bottomed 96-well plate at 2×10⁴/well, and cultured for three days together with 2×10⁴ splenic CD11c⁺ cells (antigen-representing cells) subjected to 30 Gy radiation irradiation treatment, 0.5 μg/ml of an anti-CD3 antibody, and a lot of Treg cells. In addition, for the last six hours, the CD4⁺ CD25⁻ cells were cultured, with [³H]-thymidine (1 μCi/well) was added thereto. Note that, Treg cells used in Example 14 were CD4⁺ GFP⁺ T cells isolated from the spleen of Foxp3^{eGFP} reporter mice, or CD4⁺ Venus⁺ T cells in the colonic lamina propria of GF Il10^{venus} mice in which Clostridium spp. were colonized or SPF Il10^{venus} mice. Then, proliferation of the cells was determined based on the uptake amount of [³H]-thymidine, and represented by a count per minute (cpm) value.

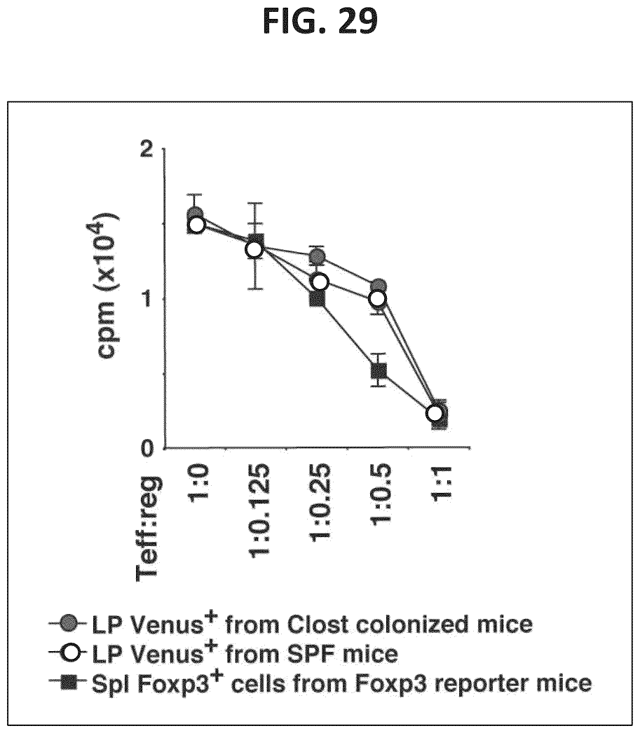

As is apparent from the results shown in FIG. 29, Venus⁺ CD4⁺ cells of the mice in which the genus Clostridium was colonized suppressed in vitro proliferation of CD25⁻ CD4⁺ activated T cells. The suppression activity was slightly inferior to that of GFP⁺ cells isolated from the Foxp3^{eGFP} reporter mice, but equal to that of Venus⁺ cells isolated from the SPF Il10^{Venus} mice. Accordingly, it has been shown that the genus Clostridium induces IL-10-expressing T cells having sufficient immunosuppressive activities, and thereby plays a critical role in maintaining immune homeostasis in the colon.

### Example 15

Next, the influence, on the local immune response, of the colonization of a large number of Clostridium and the resultant proliferation of Treg cells was investigated.

### Dextran Sulfate Sodium (DSS)-Induced Colitis Model

First, the DSS-induced colitis model was prepared as described above, and the influence, on the model mice, of the inoculation of the Clostridium and the proliferation of Treg cells was investigated. Specifically, control mice and Clostridium-inoculated mice were treated with 2% DSS, then observed and measured for six days for the body weight loss, the hardness of stool, and bleeding, and then were evaluated numerically. In addition, on day 6, the colons were collected, dissected, and analyzed histologically by HE staining. FIGS. 41 to 43 show the obtained results. Note that, in FIGS. 41 to 43, “SPF+Clost.” or “SPF+Clost. #1 to 3” indicate the results of C57BL/6 mice inoculated with a fecal suspension of Clostridium-colonized mice, and grown in a conventional environment for six weeks, and “SPF” or “SPF #1 to 3” indicate the results of C57BL/6 mice (control mice) grown in a conventional environment for six weeks without being inoculated with the fecal suspension. In addition, in FIG. 41, the vertical axis “Disease score” represents the disease activity index (DAI) described above, and the horizontal axis “post 2% DSS(d)” represents the days elapsed after the initial administration of 2% DSS to the mice. Moreover, in FIG. 41, * indicates that “P<0.02,” and ** indicates that “P<0.001.” Meanwhile, Treg cells induced by regulatory dendritic cells are known to play a preventive role in a DSS-induced colitis model (see S. Manicassamy et al., Science 329, 849 (Aug. 13, 2010)).

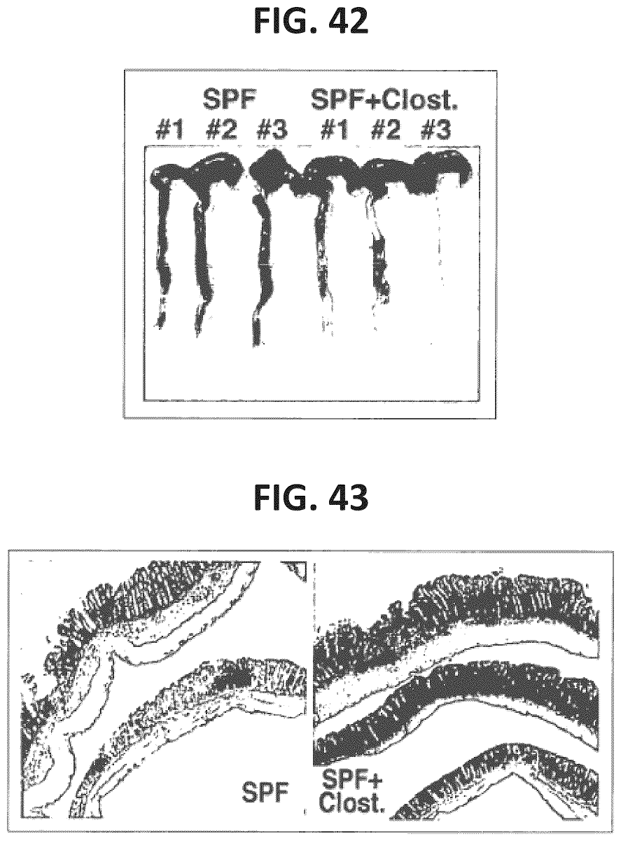

As is apparent from the results shown in FIGS. 41 to 43, the symptoms of the colitis such as body weight loss and rectal bleeding were significantly suppressed in the mice having a large number of Clostridium (hereinafter also referred to as “Clostridium-abundant mice”) in comparison with the control mice (see FIG. 41). All the features typical for colonic inflammation, such as shortening of the colon, edema, and hemorrhage, were observed markedly in the control mice in comparison with the Clostridium-abundant mice (see FIG. 42). Moreover, histological features such as mucosal erosion, edema, cellular infiltration, and crypt loss were less severe in the DSS-treated Clostridium-abundant mice than in the control mice (see FIG. 43).

### Oxazolone-Induced Colitis Model

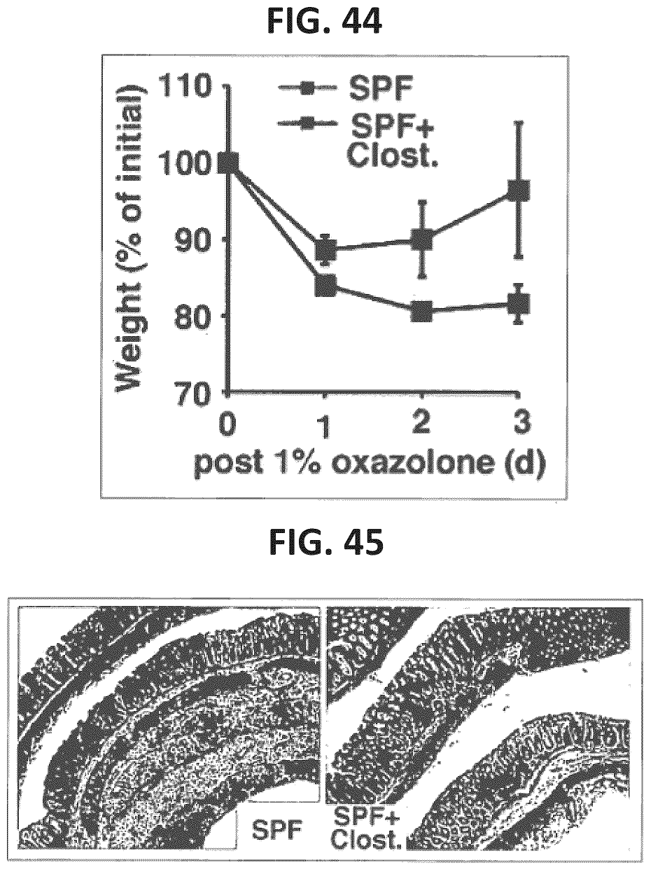

Next, the oxazolone-induced colitis model was prepared as described above, and the influence, on the model mice, of the inoculation of Clostridium and the proliferation of Treg cells was investigated. Specifically, control mice and Clostridium-inoculated mice were sensitized with oxazolone, and subsequently the inside of the rectums thereof were treated with a 1% oxazolone/50% ethanol solution. Then, the body weight loss was observed and measured. In addition, the colons were dissected, and analyzed histologically by HE staining. FIGS. 44 and 45 show the obtained results. Note that, in FIGS. 44 and 45, “SPF+Clost.” indicates the results of C57BL/6 mice (Clostridium-abundant mice) inoculated with a fecal suspension of Clostridium-colonized mice, and grown in a conventional environment for six weeks, and “SPF” indicates the results of C57BL/6 mice (control mice) grown in a conventional environment for six weeks without being inoculated with the fecal suspension. In addition, in FIG. 44, the vertical axis “Weight (% of initial)” represents the body weight after the administration of 1% oxazolone where the body weight before the administration was taken as 100%, and the horizontal axis “post 1% oxazolone (d)” represents the days elapsed after the administration of 1% oxazolone to the mice. Meanwhile, it is known that Th2-type T cells are involved in colitis induced by oxazolone. (see M. Boirivant, I. J. Fuss, A. Chu, W. Strober, J Exp Med 188, 1929 (Nov. 16, 1998)).

As is apparent from the results shown in FIGS. 44 and 45, the colitis proceeded along with persistent body weight loss in the control mice. Meanwhile, the body weight loss of the Clostridium-abundant mice was reduced (see FIG. 44). In addition, it was also revealed that portions having histological diseases such as mucosal erosion, edema, cellular infiltration, and hemorrhage were reduced in the colon of the Clostridium-abundant mice (see FIG. 45).

### Example 16

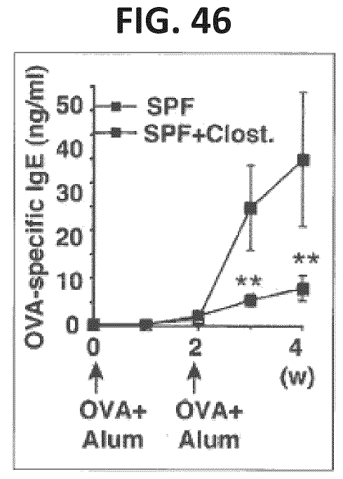

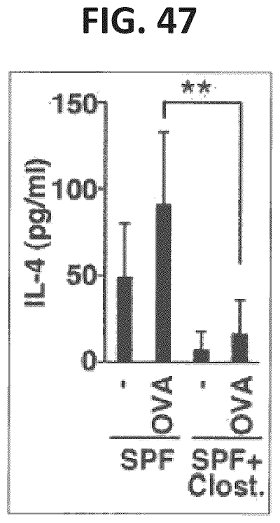

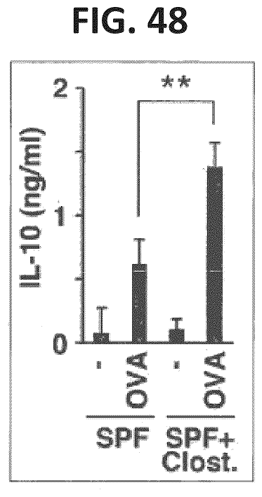

Next, the influence, on the systemic immune response (systemic IgE production), of the colonization of a large number of Clostridium and the resultant proliferation of Treg cells was investigated. Specifically, as described above, control mice and Clostridium-inoculated mice were immunized by administering alum-absorbed ovalbumin (OVA) twice at a 2-week interval. Then, sera were collected from these mice, and the OVA-specific IgE level thereof was investigated by ELISA. In addition, splenic cells were collected from the mice in each group, and IL-4 and IL-10 production by in-vitro OVA restimulation was investigated. FIGS. 46 to 48 show the obtained results. Note that, in FIGS. 46 to 48, “SPF+Clost.” indicates the results of BALB/c SPF mice (Clostridium-abundant mice) inoculated with a fecal suspension of Clostridium-colonized mice, and grown in a conventional environment, “SPF” indicates the results of BALB/c SPF mice (control mice) grown in a conventional environment without being inoculated with the fecal suspension, and ** indicates that “P<0.001.” Meanwhile, in FIG. 46, the vertical axis “OVA-specific IgE (ng/ml)” represents the concentration of OVA-specific IgE in the sera. Moreover, in FIG. 46, the horizontal axis represents the days elapsed after the initial administration of the alum-absorbed ovalbumin to the Clostridium-abundant mice or the control mice (4-week old), and “OVA+Alum” indicates the timing of the administration of the alum-absorbed ovalbumin. In addition, in FIGS. 47 and 48, “OVA” on the horizontal axis indicates the results in the case where the in-vitro OVA restimulation was performed, and “−” indicates the results in the case where no in-vitro OVA restimulation was performed. Moreover, in FIGS. 47 and 48, the vertical axes “IL-4 (pg/ml)” and “IL-10 (pg/ml)” show the IL-4 concentration and the IL-10 concentration in culture supernatants of splenic cells, respectively.

As is apparent from the results shown in FIGS. 46 to 48, the IgE level was significantly lower in the Clostridium-abundant mice than in the control mice (see FIG. 46). Moreover, the IL-4 production by the OVA restimulation was reduced (see FIG. 47) and the IL-10 production thereby was increased (see FIG. 48) in the splenic cells of the Clostridium-abundant mice sensitized with OVA and alum, in comparison with those of the control mice.

Accordingly, in consideration of the results shown in Example 15 in combination, it has been revealed that the induction of Treg cells by Clostridium in the colon plays an important role in local and systemic immune responses.

### Example 17

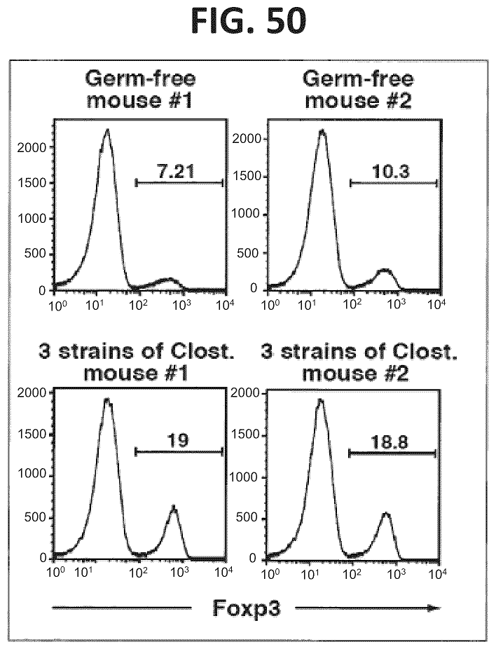

Next, GF Balb/c were colonized with three strains of Clostridium belonging to cluster IV(strains 22, 23 and 32 listed in FIG. 49). Three weeks later, colonic Foxp3⁺ Treg cells were analyzed by FACS. FIG. 50 shows the obtained results. As is apparent from the results shown in FIG. 50, gnotobiotic mice colonized with three strains of Clostridium showed an intermediate pattern of Treg induction between GF mice and mice inoculated with all 46 strains.

### Example 18

Next, it was investigated whether or not a spore-forming (for example, a chloroform resistant) fraction of a fecal sample obtained from humans had the effect of inducing proliferation or accumulation of regulatory T cells similar to the spore-forming fraction of the fecal sample obtained from mice.

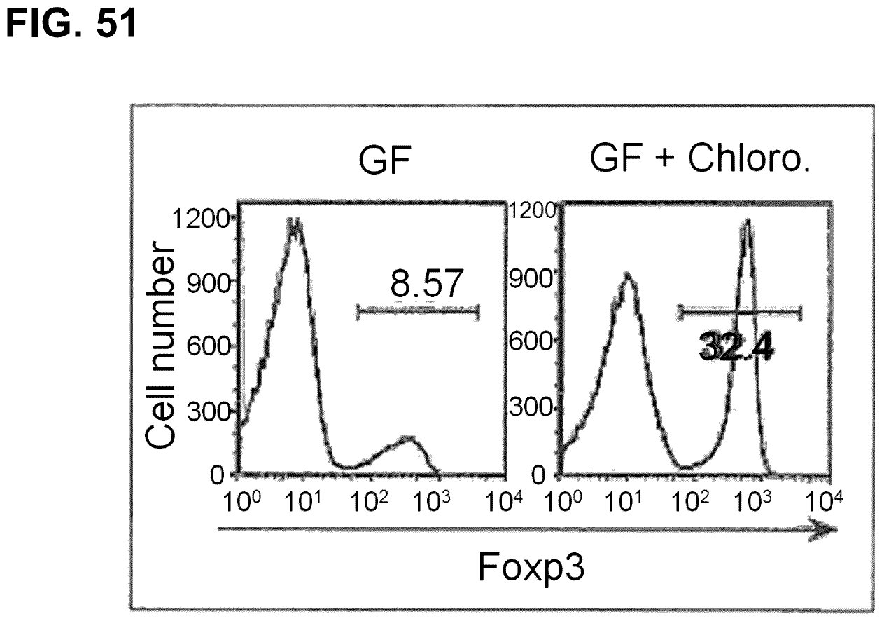

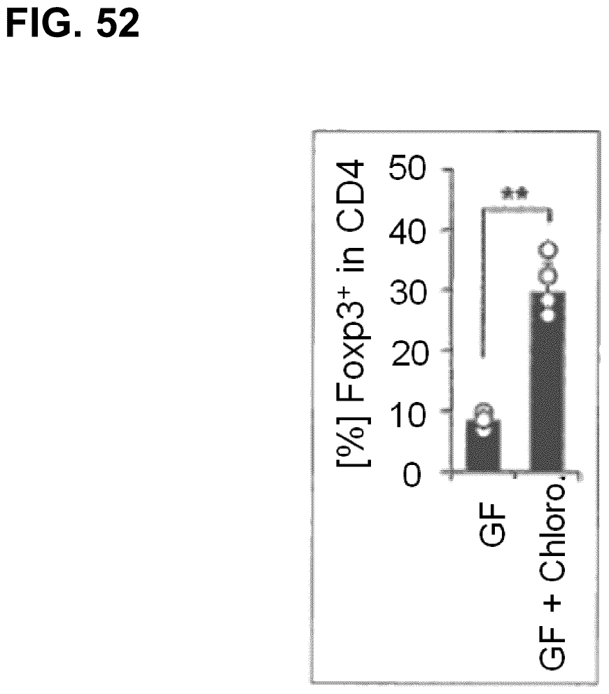

Specifically, human stool from a healthy volunteer (Japanese, male, 29 years old) was suspended with phosphate-buffered saline (PBS), mixed with chloroform (final concentration 3%), and then incubated in a shaking water bath for 60 min. After evaporation of chloroform by bubbling with N₂ gas, the aliquots containing chloroform-resistant (for example, spore-forming) fraction of human intestinal bacteria were orally inoculated into germ-free (GF) mice (IQI, 8 weeks old). The treated mice were kept in a vinyl isolator for 3 weeks. The colon was collected and opened longitudinally, washed to remove fecal content, and shaken in Hanks' balanced salt solution (HBSS) containing 5 mM EDTA for 20 min at 37° C. After removing epithelial cells and fat tissue, the colon was cut into small pieces and incubated with RPMI1640 containing 4% fetal bovine serum, 1 mg/ml collagenase D, 0.5 mg/ml dispase and 40 μg/ml DNase I (all manufactured by Roche Diagnostics) for 1 hour at 37° C. in a shaking water bath. The digested tissue was washed with HBSS containing 5 mM EDTA, resuspended in 5 ml of 40% PERCOLL® (manufactured by GE Healthcare) and overlaid on 2.5 ml of 80% PERCOLL® in a 15-ml Falcon tube. PERCOLL® gradient separation was performed by centrifugation at 780 g for 20 min at 25° C. The interface cells were collected and suspended in staining buffer containing PBS, 2% FBS, 2 mM EDTA and 0.09% NaN₃ and stained for surface CD4 with Phycoerythrin-labeled anti-CD4 Ab (RM4-5, manufactured by BD Biosciences). Intracellular staining of Foxp3 was performed using the Alexa647-labeled anti-Foxp3 Ab (FJK-16s, manufactured by eBioscience) and Foxp3 Staining Buffer Set (manufactured by eBioscience). The percentage of Foxp3 positive cells within the CD4 positive lymphocyte population was analyzed by flow cytometry. FIGS. 51 and 52 show the obtained results.

In figures, representative histograms (FIG. 51) and combined data (FIG. 52) for Foxp3 expression by CD4 positive lymphocytes from GF mice (GF) or GF mice gavaged with chloroform-treated human stool (GF+Chloro.) are shown. In addition, numbers in FIG. 51 indicate the percentages of cells in the gate. Each circle in FIG. 52 represents a separate animal, error bars indicate the SD, and ** indicates that “P<0.001.”

As is apparent from the results shown in FIGS. 51 and 52, it was found that also when the spore-forming(for example, the chloroform resistant) fraction of human intestinal bacteria was colonized in GF mice, the accumulation of Foxp3⁺ regulatory (Treg) cells in the colonic lamina propria of the mice was induced.

Next, it was investigated what species of bacteria grew by gavaging with chloroform-treated human stool.

Specifically, using a QIAamp DNA Stool mini kit (manufactured by QIAGEN), bacterial genomic DNA was isolated from the human stool from a healthy volunteer as described above (human stool) or fecal pellets from GF mice gavaged with chloroform-treated human stool (GF+Chloro.). Quantitative PCR analysis was carried out using a LightCycler 480 (manufactured by Roche). Relative quantity was calculated by the ΔCt method and normalized to the amount of total bacteria, dilution, and weight of the sample. The following primer sets were used:

total bacteria
(SEQ ID NO: 62)
5′-GGTGAATACGTTCCCGG-3′
and
(SEQ ID NO: 63)
5′-TACGGCTACCTTGTTACGACTT-3′
Clostridium cluster XIVa
(Clostridium coccoides subgroup)
(SEQ ID NO: 64)
5′-AAATGACGGTACCTGACTAA-3′
and
(SEQ ID NO: 65)
5′-CTTTGAGTTTCATTCTTGCGAA-3′
Clostridium cluster IV (Clostridium leptum)
(SEQ ID NO: 66)
5′-GCACAAGCAGTGGAGT-3′
and
(SEQ ID NO: 24)
5′-CTTCCTCCGTTTTGTCAA-3′
Bacteroides
(SEQ ID NO: 67)
5′-GAGAGGAAGGTCCCCCAC-3′
and
(SEQ ID NO: 68)
5′-CGCTACTTGGCTGGTTCAG-3′.

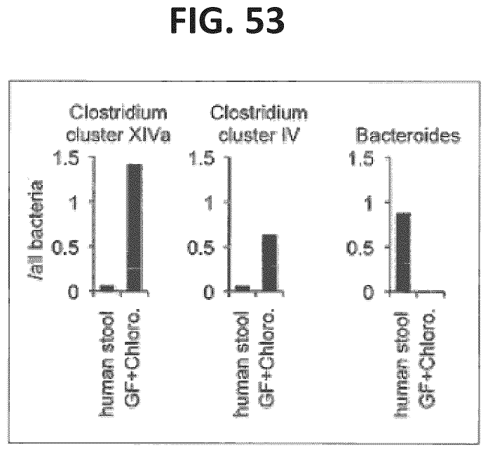

FIG. 53 shows the obtained results.

As is apparent from the results shown in FIG. 53, mice gavaged with chloroform-treated human stool exhibited high amounts of spore-forming bacteria, such as Clostridium clusters XIVa and IV, and a severe decrease of non-spore-forming bacteria, such as Bacteroides, compared with the human stool before chloroform treatment.

### Industrial Applicability

As has been described above, the present invention makes it possible to provide an excellent composition for inducing proliferation or accumulation of regulatory T cells (Treg cells) by utilizing bacteria belonging to the genus Clostridium or a physiologically active substance or the like derived from the bacteria. Since the composition of the present invention has immunosuppressive effects, the composition can be used, for example, to prevent or treat autoimmune diseases or allergic diseases, as well as to suppress immunological rejection in organ transplantation or the like. In addition, healthy individuals can easily and routinely ingest the composition as a food or beverage, such as a health food, to improve their immune functions.

### Sequence Listing

SEQ ID NO:1 to 20, 62 to 69

<223> Artificially synthesized primer sequence

SEQ ID NO:21 to 61

<223> 16S rRNA coding gene sequence of each Clostridium strain

What is claimed is:

1. A pharmaceutical composition comprising a spore-forming fraction of human fecal matter;
wherein the pharmaceutical composition is formulated for oral administration;
wherein the pharmaceutical composition prevents an infectious disease in a human subject; and
wherein the spore-forming fraction of human fecal matter in the pharmaceutical composition induces proliferation and/or accumulation of regulatory T cells.

2. The pharmaceutical composition of claim 1, wherein the infectious disease is a Clostridium difficile infection.

3. The pharmaceutical composition of claim 2, wherein the infectious disease is a recurrence of Clostridium difficile infection.

4. The pharmaceutical composition of claim 3, wherein the pharmaceutical composition is configured to be administered after antibiotic treatment.

5. The pharmaceutical composition of claim 1, wherein the pharmaceutical composition is in the form of a capsule.

6. The pharmaceutical composition of claim 1, wherein the pharmaceutical composition is formulated for delivery to the intestine of the human subject.

7. The pharmaceutical composition of claim 1, wherein the pharmaceutical composition is formulated for delivery to the colon of the human subject.

8. The pharmaceutical composition of claim 5, wherein the capsule comprises a hydrogel.

9. The pharmaceutical composition of claim 8, wherein the hydrogel comprises a cellulose-based polymer.

10. The pharmaceutical composition of claim 5, wherein the capsule comprises a pH-sensitive composition comprising one or more enteric polymers.

11. The pharmaceutical composition of claim 1, wherein the regulatory T cells are Foxp3+ regulatory T cells.

12. The pharmaceutical composition of claim 1, wherein the pharmaceutical composition does not include bacteria belonging to the genus Bacteroides.


SEQUENCE LISTING (69 sequences)

SEQ ID NO: 1 - DNA, 23 bp, synthetic construct
  misc_feature 1..23: note = Artificially synthesized primer sequence
  source 1..23: mol_type = other DNA
ggcaatagttccttcccagagtt

SEQ ID NO: 2 - DNA, 22 bp, synthetic construct
  misc_feature 1..22: note = Artificially synthesized primer sequence
  source 1..22: mol_type = other DNA
gggtcgcatattgtggtacttg

SEQ ID NO: 3 - DNA, 23 bp, synthetic construct
  misc_feature 1..23: note = Artificially synthesized primer sequence
  source 1..23: mol_type = other DNA
ccttttgtagccctgctcactct

SEQ ID NO: 4 - DNA, 21 bp, synthetic construct
  misc_feature 1..21: note = Artificially synthesized primer sequence
  source 1..21: mol_type = other DNA
gggtcacctgtatggcttcag

SEQ ID NO: 5 - DNA, 21 bp, synthetic construct
  misc_feature 1..21: note = Artificially synthesized primer sequence
  source 1..21: mol_type = other DNA
tcagtgcaagatctgcaagca

SEQ ID NO: 6 - DNA, 19 bp, synthetic construct
  misc_feature 1..19: note = Artificially synthesized primer sequence
  source 1..19: mol_type = other DNA
acaccggaagccaaacaca

SEQ ID NO: 7 - DNA, 27 bp, synthetic construct
  misc_feature 1..27: note = Artificially synthesized primer sequence
  source 1..27: mol_type = other DNA
gattttaataagctccaagaccaaggt

SEQ ID NO: 8 - DNA, 27 bp, synthetic construct
  misc_feature 1..27: note = Artificially synthesized primer sequence
  source 1..27: mol_type = other DNA
cttctatgcagttgatgaagatgtcaa

SEQ ID NO: 9 - DNA, 21 bp, synthetic construct
  misc_feature 1..21: note = Artificially synthesized primer sequence
  source 1..21: mol_type = other DNA
cctcgtcccgtagacaaaatg

SEQ ID NO: 10 - DNA, 20 bp, synthetic construct
  misc_feature 1..20: note = Artificially synthesized primer sequence
  source 1..20: mol_type = other DNA
tctccactttgccactgcaa

SEQ ID NO: 11 - DNA, 20 bp, synthetic construct
  misc_feature 1..20: note = Artificially synthesized primer sequence
  source 1..20: mol_type = other DNA
ggacattgtctttgatggca

SEQ ID NO: 12 - DNA, 20 bp, synthetic construct
  misc_feature 1..20: note = Artificially synthesized primer sequence
  source 1..20: mol_type = other DNA
cttgtcacgtggtgtcactg

SEQ ID NO: 13 - DNA, 20 bp, synthetic construct
  misc_feature 1..20: note = Artificially synthesized primer sequence
  source 1..20: mol_type = other DNA
tctctggacgtcaaatgtgg

SEQ ID NO: 14 - DNA, 19 bp, synthetic construct
  misc_feature 1..19: note = Artificially synthesized primer sequence
  source 1..19: mol_type = other DNA
gctgaacagcagagccttc

SEQ ID NO: 15 - DNA, 20 bp, synthetic construct
  misc_feature 1..20: note = Artificially synthesized primer sequence
  source 1..20: mol_type = other DNA
aggtctggatcactccaagg

SEQ ID NO: 16 - DNA, 19 bp, synthetic construct
  misc_feature 1..19: note = Artificially synthesized primer sequence
  source 1..19: mol_type = other DNA
tcgcctggaccataaagaa

SEQ ID NO: 17 - DNA, 20 bp, synthetic construct
  misc_feature 1..20: note = Artificially synthesized primer sequence
  source 1..20: mol_type = other DNA
agaggatgcgtgactttgtg

SEQ ID NO: 18 - DNA, 20 bp, synthetic construct
  misc_feature 1..20: note = Artificially synthesized primer sequence
  source 1..20: mol_type = other DNA
atacagcagaccttctggca

SEQ ID NO: 19 - DNA, 20 bp, synthetic construct
  misc_feature 1..20: note = Artificially synthesized primer sequence
  source 1..20: mol_type = other DNA
agagtttgatcmtggctcag

SEQ ID NO: 20 - DNA, 16 bp, synthetic construct
  misc_feature 1..16: note = Artificially synthesized primer sequence
  source 1..16: mol_type = other DNA
attaccgcggckgctg

SEQ ID NO: 21 - DNA, 1460 bp, Clostridium leptum
  rRNA 1..1460: note = 16S rRNA coding gene sequence of Clostridium strain 1
  source 1..1460: mol_type = genomic DNA
agagtttgatcctggctcaggatgaacgctggcggcgtgcttaacacatgcaagtcgaacgagaaccattggatcgaggattcgtccaagtgaaggtggggaaagtggcggacgggtgagtaacgcgtgagcaatctgccttggagtggggaataacggctggaaacagccgctaataccgcatgatacagctgggaggcatctccctggctgtcaaagatttatcgctctgagatgagctcgcgtctgattagctagttggcggggtaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttgtcagggacgaagcaagtgacggtacctgacacggctactacggtcagcagcgcgtatacgtaggtgccagcgtatccggaattacctgggttaaggcgtgttagccggactgcagtcagatgtgaatcacgggctcaacttgtgctgcattggaactgtagttctgagtactgagagcagacggaattctaggtagcggtgaatgcgtagatataggaggacacagtgcgaggcgtctgctgacagcaactgacgctgaggcgggaagcgtgggggagccaacaggattagatacctggtagttcacgcctggtaaaacgatggatactaggtgtggggggactgaccccctcgtggccgccagttaacaccaataaagtatcccacctgggagtacgatcgcaaggttgaaactcaaaggaattgacggggcccgcacaagcggtggagtatgtggtttaattcgaagcaacgcgaagaaccttaccagggcttgacatcccgaggaccggactagagatagtcttttctcttcggagacctcggtgacaggtggtgcatggttgtcgtcagctcgtgtcgtaagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggtcaacagagggaagcaataccgcgaggtggagcaaatccctaaaagccatcccagttcggatcgcaggctgcaacccgcctgcgtgaagttggaatcgctagtaatcgcggatcagcatgccgcggtgaatacgttcccgggccttgtacacaccgcccgtcacaccatgagagtcgggaacacccgaagtccgtagcctaaccgcaagggggggcgcggccgaaggtgggttcgataattggggtgaagtcgtaacaaggtagccgt

SEQ ID NO: 22 - DNA, 1485 bp, Clostridium leptum
  rRNA 1..1485: note = 16S rRNA coding gene sequence of Clostridium strain 2
  source 1..1485: mol_type = genomic DNA
agagtttgatcctggctcaggatgaacgctggcggcgtgcttaacacatgcaagtcgaacggagcacccctgaaggagttttcggacaacggaagggactgcttagtggcggacgggtgagtaacgcgtgaggaacctgccttggagtggggaataacagctggaaacagctgctaataccgcataatatatctgggccgcatggctctggatatcaaagatttatcgctctgagatggactcgcgtctgattagctagttggcggggtaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttgtcagggacgaagcaagtgacggtacctgacgaataagccacggctaactacgtgccagcagccgcggtaatacgtaggtggcaagcgttatccggatttactgggtgtaaagggcgtgtaggcgggactgcaagtcagatgtgaaaaccacgggctcaacctgtgggcctgcatttgaaactgtagttcttgagtactggagaggcagacggaattctagttgtagcgtgaaatgcgtagatatagaagaacacagttgcggagccggtctgcaactgacgctgagcgcgaaagcgtggggagcaaacaggattagataccctggtagtccacgctgtaaacgatggattactaggtgtggggggactgaccccctccgtgccgcagttaacacaataagtatcccacctggggagtacgatcgcaaggttgaaactcaaaaggaattgacgggggcccgcacaagcggtggagtatgtggtttaaattcgaagcaacgcgaagaaccttaccagggcttgacatcccggtgaccgtcctagagataggattttcccttcggggacactggagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggccacacacgtactacaatggtggtcaacagagggaagcaaagccgcgaggtggagcaaatccctaaaagccatcccagttcggatcgcaggctgcaacccgcctgcgtgaagttggaatcgctagtaatcgcggatcagaatgccgcggtgaatacgttcccgggccttgtacacaccgcccgtcacaccatgagagtcgggaacacccgaagtccgtagcctaaccgcaaggggggcgcggccgaaggtgggttcgataattggggtgaagtcgtaacaaggtagccgt

SEQ ID NO: 23 - DNA, 1491 bp, Clostridium leptum
  rRNA 1..1491: note = 16S rRNA coding gene sequence of Clostridium strain 3
  source 1..1491: mol_type = genomic DNA
agagtttgatcctggctcaggacgaacgctggcggcgtgcttaacacatgcaagtcgaacggagcaccttcgaaagagacttcggtcaatggaaaagaatgcttagtggcggacgggtgagtaacgcgtgaggaacctgcctttcagtgggggacaacagttggaaacgactgctaataccgcataacgtacgggtatcgcatggtatctgtaccaaagatttatcgctgagagatggcctcgcgtctgattagctagttggtagggtaacggcctaccaaggcgacgatcagtagccggactgagaggttggccggccacattgggactgagatacggcccagactcctacgggaggcagcagtggggaatattgggcaatgggcgaaagcctgacccagcaacgccgcgtgaaggaagaaggctttcgggttgtaaacttcttttgacggggaagagcagaagacggtacctgtcgaataagccacggctaactacgtgccagcagccgcggtaatacgtaggtggcaagcgttgtccggatttactgggtgtaaagggcgtgtagccgggctgacaagtcagatgtgaaatccgggggctcaacccccgaactgcatttgaaactgttggtcttgagtatcggagaggcaggcggaattcctagtgtagcggtgaaatgcgtagatattagggggaacaccagtggcgaagcggcctgctggacgacaactgacggtgaggcgcgaaagcgtggggagcaaacaggattagataccctggtagtccacgctgtaaacgatggatactaggtgtgcggggactgacccctgcgtgccgcagctaacgcaataagtatcccacctggggagtacgatcgcaaggttgaaactcaaaggaattgacgggggcccgcacaagcggtggattatgtggtttaattcgatgcaacgcgaagaaccttaccagggcttgacatcctactaacgaagtagagatacattaggtacccttcgggggaagtagagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggctacacacgtaatacaatggcggtcaacagagggatgcaaaaccgcgaggtggagcgaacccctaaaagccgtcccagttcagatcgcagtctgcaacccgactgcgtgaagtcggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgt

SEQ ID NO: 24 - DNA, 1491 bp, Blautia coccoides
  rRNA 1..1491: note = 16S rRNA coding gene sequence of Clostridium strain 4
  source 1..1491: mol_type = genomic DNA
agagtttgatcctggctcaggatgaacgctggcggcgtgcctaacacatgcaagtcgaacgggtgtacggggaggaaggcttcggccggaaaacctgtgcatgagtggcggacgggtgagtaacgcgtgggcaacctggcctgtacagggggataacacttagaaataggtgctaataccgcataacgggggaagccgcatggcttttccctgaaaactccggtggtacaggatgggcccgcgtctgattagccagttggcagggtaacggcctaccaaagcgacgatcagtagccggcctgagagggcggacggccacactgggactgagacacggcccagactcctacgggaggcagcagtgggggatattgcacaatggggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcagcgcaagtctgagtgaaatcccatggcttaaccatggaactgctttggaaactgtgcagctggagtgcaggagagtaagcggaattcctagtgtagcgtgaaatgcgtagattataggaggaacaccagtggcgaaggcggctaactgaactgtaactgacgttgaggctcgaaagcgtggggagcaaacaggattagataccctggtagtccacgccgtaaacgatgattactaggtgttgggggaccaaggtcttcggtgccggcgcaaacgcattaagtaatccacctggggagtacgttcgcaagaatgaaactcaaaggaattgacggggacccgcacaagcggtggagcatgtggtttaattcgaagcaacgcgaagaaccttacctggtcttgacatcccgatgacgagtgagcaaagtcactttcccttcggggcattggagacaggtggtgcatggttgtcgtcagctcgtgtcgtgagatgttgggttaagtcccgcaacgagcgcaacccctatttccagtagccagcaggtagagctgggcactctggagagactgcccgggataaccgggaggaaggcggggatgacgtcaaatcatcatgccccttatgatcagggctacacacgtgctacaatggcgtaaacaaagggaagcgagacggtgacgttgagcaaatcccaaaaataacgtcccagttcggattgtagtctgcaactcgactacatgaagctggaatcgctagtaatcgcgaatcagaatgtcgcggtgaatacgttcccgggtcttgtacacaccgcccgtcacaccatgggagtcggaaatgcccgaagtcagtgacctaaccgaaaggaaggagctgccgaaggtggagccggtaactggggtgaagtcgtaacaaggtagccgt

SEQ ID NO: 25 - DNA, 1467 bp, Clostridium leptum
  rRNA 1..1467: note = 16S rRNA coding gene sequence of Clostridium strain 5
  source 1..1467: mol_type = genomic DNA
agagtttgatcctggctcaggacgaacgctggcggcacgcctaacacatgcaagtcgaacggagtgaagatgctcgcatctgaacttagtggcggacgggtgagtaacacgtgagcaacctgcctttcagagggggattacgtttggaaacgaacgctaataccgcataaaatatcggagtcgcatggcactgatatcaaaggagcaatccgctgaaagatgggctcgcgtccgattaggcagttggcggggtatcggcccaccaaaccgacaatcggtagccggactgagaggttgaacggccacattgggactgagacgcggcccagactcctacgggaggcagcagtgggggatattgcacaatgggggaaaccctgatgcagcgatgccgcgtgaatgaagacggccttcgggttgtaaagttctgtcgcaggggacgaaaatgacggtaccctgcaagaaagctccggctaactacgtgccagcagccgcggtaatacgtagggagcaagcgttgtccggaattactgggtgtaaagggagcgtaggcgggaggataagttgaatgtgaaatctatgggctcaacccatagctgcgttcaaactgttcttcttgagtgaagtagaggcaggcggaattcctagtgtagcggtgaaatgcgtagatattaggaggacaccagtggcgaaggcgggctgctgggctttactgacgctgaggctcgaaagcgtgggtagcaaacaggattagataccctggtagtccacgcggtaaacgatgattactaggtgtgggtggactgaccccatccgtgccggagttaacacaataagtaatccacctggggagtacggccgcaaggttgaaactcaaaggaattgacgggggcccgcacaagcagtggagtatgtggtttaattcgacgcaacgcgaagaaccttaccaggtcttgacatcgagtgacggacatagagatatgtctttccttcgggacacgaagacaggtggtgcatggttgtcgtcagctcgtgtcgtgagatgttgggttaagtcccgcaacgagcgcaacccttaccattagttgctacgcaagagcactctaatgggactgccgttgacaaaacggaggaaggtggggatgacgtcaaatcatcatgccccttatgacctgggcgacacacgtactacaatggcggtcaacagagggaggcaaagccgcgaggcagagcaaacccctaaaagccgtctcagttcggattgcaggctgcaactcgcctgcatgaagtcggaattgctagtaatcgcggatcagcatgccgcggtgaatacgttcccgggccttgtacacaccgcccgtcacaccatgagagccggtaacacccgaagtcaatagtctaaccgcaaggaggacattgccgaaggtgggattggtaattggggtgaagtcgtaacaaggtagccgt

SEQ ID NO: 26 - DNA, 1474 bp, Blautia coccoides
  rRNA 1..1474: note = 16S rRNA coding gene sequence of Clostridium strain 6
  source 1..1474: mol_type = genomic DNA
agagtttgatcatggctcaggatgaacgctggcggcgtgcctaacacatgcaagtcgaacgggtgtacgggaaggaaggcttcggccggaaaacctgtgcatgagtggcggacgggtgagtaacgcgtgggcaacctggcctgtacagggggataacacttagaaataggtgctaataccgcataacgggggaagccgcatggcttttccctgaaaactccggtggtacaggatgggcccgcgtctgattagccagttggcagggtaacggcctaccaaagcgacgatcagtagccggcctgagagggcggacggccacactgggactgagacacggcccagactcctacgggaggcagcagtgggggatattgcacaatggggggaaccctgatgcagcgacgccgcgtgggtgaagaagcgcctcggcgcgtaaagccctgtcagcagggaagaaaatgacggtacctgaagaagaagccccggctaactacgtgccagcagccgcggtaatacgtaggggggcaagcgttatccggatttactgggtgtaaagggggcgcagacggcgatgcaagccaggagtgaaagcccggggcccaaccccgggactgctcttgggaactgcgtggctggagtgcagagggcagcggaattcctggtgaaatgcgtagatatcagaagacacggtgcgaggcggcctgctgactgcactgacgttgagccgaagcgtggggagcaaacaggattagataccgtggtagtcacgccgtaaacgatgattactaggtgtcggggagcagagactgcccggtgccgcagccaacgcattaagtaatccacctggggagtacgttcgcaagaatgaaactcaaaggaattgacggggacccgcacaagcggtggagcatgtggtttaattcgaagcaacgcgaagaaccttacctggtcttgacatcccgatgacgagtgagcaaagtcactttcccttcggggcattggagacaggtggtgcatggttgtcgtcagctcgtgtcgtgagatgttgggttaagtcccgcaacgagcgcaacccctatttccagtagccagcaggtagagctgggcactctggagagactgcccgggataaccgggaggaaggcggggatgacgtcaaatcatcatgccccttatgatcagggctacacacgtgctacaatggcgtaaacaaagggaagcgagacggtgacgttaagcaaatcccaaaaataacgtcccagttcggattgtagtctgcaactcgactacatgaagctggaatcgctagtaatcgcgaatcagaatgtcgcggtgaatacgttcccgggtcttgtacacaccgcccgtcacaccatgggagtcggaaatgcccgaagtcagtgacctaaccgaaaggaaggagctgccgaaggtggagccggtaactggggtgaagtcgtaacaaggtagccgt

SEQ ID NO: 27 - DNA, 1484 bp, Clostridium leptum
  rRNA 1..1484: note = 16S rRNA coding gene sequence of Clostridium strain 7
  source 1..1484: mol_type = genomic DNA
agagtttgatcctggctcaggatgaacgctggcggcgtgcttaacacatgcaagtcgaacgagaatccagtgaaggagttttcggacaacggatctggaggaaagtggcggacgggtgagtaacgcgtgagcaatctgccttggagtggggaataacggttggaaacagccgctaataccgcatgatgcgtctgggaggcatctctctggacgccaaagatttatcgctctgagatgagctcgcgtctgattagcttgttggcggggtaaaggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttctgagggacgaagaaagtgacggtacctcaggaataagccacggctaactacgtgccagcagccgcggtaatacgtaggtggcaagcgttatccggatttattgggtgtaaagggcgtgtaggcgggaaagcaagtcagatgtgaaaactcagggctcaaccctgagcctgcatttgaaactgtttttcttgagtgctggagaggcaatcggaattccgtgtgtagcggtgaaatgcgtagatatacggaggacaccagtggcgagcggattgctggacagtactgacgctgaagcgcgaaagcgtgggagcaaacagatagatacctggtagtcacgcgtaaacgatggatactaggtgtggggggactgaccccctccgtgccgcagctaacgcaataagtatcccacctggggagtacgatcgcaaggttgaaactcaaaggaattgacgggggcccgcacaagcggtggagtatgtggtttaattcgaagcaacgcgaagaaccttaccagggcttgacatcctgctaacgaaccagagatggattaggtgcccttcggggaaagcagagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttacgtcctgggccacacacgtactacaatggcggccaacaaagagaggcaagaccgcgaggtggagcaaatctcaaaaagccgtcccagttcggatcgcaggctgcaacccgcctgcgtgaagttggaatcgctagtaatcgcggatcagcatgccgcggtgaatacgttcccgggccttgtacacaccgcccgtcacaccatgagagtcgggaacacccgaagtccgtagcctaaccgcaaggggggcgcggccgaaggtgggttcgataattggggtgaagtcgtaacaaggtagccgt

SEQ ID NO: 28 - DNA, 1483 bp, Clostridium leptum
  rRNA 1..1483: note = 16S rRNA coding gene sequence of Clostridium strain 8
  source 1..1483: mol_type = genomic DNA
agagtttgatcctggctcaggacgaacgctggcggcgtgcttaacacatgcaagtcgaacggagcacccctgaaggagttttcggacaacggatgggaatgcttagtggcggactggtgagtaacgcgtgaggaacctgccttccagagggggacaacagttggaaacgactgctaataccgcatgatgcgttggagccgcatgactccgacgtcaaagatttatcgctggaagatggcctcgcgtctgattagctagttggtgaggtaacggcccaccaaggcgacgatcagtagccggactgagaggttggccggccacattgggactgagatacggcccagactcctacgggaggcagcagtggggaatattgggcaatggacgcaagtctgacccagcaacgccgcgtgaaggaagaaggctttcgggttgtaaacttcttttaagggggaagagcagaagacggtaccccttgaataagccacggctaactacgtgccagcagccgcggtaatacgtaggtggcaagcgttgtccggatttactgggtgtaaagggcgtgcagccggagagacaagtcagatgtgaaatccacgggctcaacccgtgaactgcatttgaaactgtttcctttgagtgtcggagaggtaatcgggattccttgtgtagcggtgaatgcgtagatatagagaccacagtgccgacgccgaatactgacgatactgacggtgagcgcgaaagcgtggggagcaaacaggattagataccctggtagtccacgctgtaaacgatcgatactaggtgtgcggggactgacccctgcgtgccggagttaacacaataagtatcgcacctggggagtacgatcgcaaggttgaaactcaaaggaattgacgggggcccgcacaagcggtggattatgtggtttaattcgaagcaacgcgaagaaccttaccagggcttgacatcctgctaacgaagtagagatacattaggtgcccttcggggaaagcagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggcggggacgacgtcaaatcatcatgccccttatgtcctgggctacacacgtaatacaatggcggttaacaaagggatgcaaagccgcgaggcagagcgaaccccaaaaagccgtcccagttcggatcgcaggctgcaacccgcctgcgtgaagtcggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgt

SEQ ID NO: 29 - DNA, 1480 bp, Blautia coccoides
  rRNA 1..1480: note = 16S rRNA coding gene sequence of Clostridium strain 9
  source 1..1480: mol_type = genomic DNA
ggagtttgatcctggctcaggatgaacgctggcggcgtgcttaacacatgcaagtcgaacgggctcatattgaaacctagtgatgtatgagttagtggcggacgggtgagtaacgcgtggagaacctgccgtatactgggggataacacttagaaataggtgctaataccgcataagcgcacagcttcgcatgaagcagtgtgaaaaactccggtggtatacgatggatccgcgtctgattagctggttggcggggtaacagcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaatactgaccttacggtcagcagacggtacctgactaagaagccccgggctaactacgtgccagcagccgcggtaatacgtaggggcaagcgttatccggatttactgggtgtaaagggggcgcagacggcgatgcaagccaggagtgaaagccggggcccaaccccgggactgctcttggactgcgtggctggagtgcagagggcagcgaattcctgtgtagcgtgaatgcgtagattcagaggacacgtgcgagcgcctgctgactgcactgacgtgagcccgaagcgtggggagcaaacaggattagatacctggtagtccacgccgtaaacgatgattactaggtgtcggggagcagagactgcccggtgccgcagccaacgcattaagtaatccacctggggagtacgttcgcaagaatgaaactcaaaggaattgacggggacccgcacaagcggtggagcatgtggtttaattcgaagcaacgcgaagaaccttaccaggccttgacatccccctggatggcccgtaacggggtcagcctttcggggcaggggagacaggtggtgcatggttgtcgtcagctcgtgtcgtgagatgttgggttaagtcccgcaacgagcgcaacccctgcccgcagtagccagcattttagatggggactctgcggggactgccggggacaacccggaggaaggcggggatgacgtcaaatcatcatgccccttatggcctgggctacacacgtgctacaatggcgccgacagagggaggcgaagcggcgacgcggagcgaaccccaaaaacggcgtcccagttcggattgtagtctgcaacccgactacatgaagccggaatcgctagtaatcgcggatcagaatgccgcggtgaatacgttcccgggtcttgtacacaccgcccgtcacaccatgggagccgggaatgcccgaagtctgtgaccgaacccgtaaggggaggggcagccgaaggcaggcccggtgactggggtgaagtcgtaacaaggtagccgt

SEQ ID NO: 30 - DNA, 1489 bp, Clostridium leptum
  rRNA 1..1489: note = 16S rRNA coding gene sequence of Clostridium strain 10
  source 1..1489: mol_type = genomic DNA
agagtttgatcctggctcaggatgaacgctggcggcgtgcttaacacatgcaagtcgagcgaagcacttttttagaactcttcggagggaagagagggtgacttagcggcggacgggtgagtaacgcgtgggcaacctgccttacacagggggataacaattagaaatgattgctaataccgcataagaccacggtactgcatggtacagtggtaaaaactgaggtggtgtaagatgggcccgcgtctgattaggtagttggtggggtagaagcctaccaagccgacgatcagtagccgacctgagagggcgaccggccacattgggactgagacacggcccaaactcctacgggaggcagcagtggggaatattgcacaatgggggaaaccctgatgcagcgacgccgcgtgagtgaggaagtatttcggtatgtaaagctctatcagcagggaagaaaatgacggtacctgactaagaagcccccggctaactacgtgccagcagccgcggtaatacgtagggggcaagcgttatccggatttactgggtgtaaagggagcgtagacggacttgcaagtctgatgtgaaaatccggggcccaacccgggactgcattgaaactgtattttttggaggggtccgaggaggcaagtggaatcctgggtagcggtgaaatggcgtagaattcagggaggaacaccagtggcggaaggcgaattactggacgataactgacggtgaggcgcgaagcgtgggagcaaacaagaattagataccctggtagtcacgctgtaacgatcgatactaggtgtgcggggactgacccctgcgtgccggagttaacacaataagtatcgcactggggagtacgatcgcaaggttgaaactcaaaggaattgacgggggcccgcacaagcggtggattatgtggtttaattcgaagcaacgcgaagaaccttaccagggcttgacatcctgctaacgaagtagagatacattaggtgcccttcggggaaagcagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggcggggacgacgtcaaatcatcatgccccttatgtcctgggctacacacgtaatacaatggcggttaacaaagggatgcaaagccgcgaggcagagcgaaccccaaaaagccgtcccagttcggatcgcaggctgcaacccgcctgcgtgaagtcggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgt

SEQ ID NO: 31 - DNA, 1490 bp, Clostridium leptum
  rRNA 1..1490: note = 16S rRNA coding gene sequence of Clostridium strain 11
  source 1..1490: mol_type = genomic DNA
agagtttgatcctggctcaggatgaacgctggcggcgtgcttaacacatgcaagtcgaacgagaatccagtgaaggagttttcggacaacggatctggaggaaagtggcggacgggtgagtaacgcgtgagcaatctgccttggagtggggaataacggttggaaacagccgctaataccgcatgatgcgtctgggaggcatctctctggacgccaaagatttatcgctctgagatgagctcgcgtctgattagcttgttggcggggtaaaggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttctgagggacgaagaaagtgacggtacctcaggaataagccacggctaactacgtgccagcagccgcggtaatacgtaggtggcaagcgttatccggatttattgggtgtaaagggcgtgtaggcgggaaagcaagtcagatgtgaaaactcagggctcaaccctgagcctgcatttgaaactgtttttcttgagtgctggagaggcaatcggaattccgtgttgtagcggtgaaatgcgtagattataccggaggaaccaccagtggcggaaggcggattgctggaacagtaactgacgctgaggcgccgaaagcgtggggagcaaacaggatagataccctggtagtccacgccgtaaacgatggatactaagtgtgggggactgaccccttcgtgcccagctaagcaataagtttcccacctggggagtacgatcgcaggtgaaactcaaaggaattgacgggggcccgcccaagcgggtggagtaggggttaattggagcaacgggaagaaccttaccagggcttgacatcctgtaacgaaccagaagagggattaggtgccttcggggaaagcagagacaggtggtgcatggttgtcgtcagctcgtgtcgtgagatgtgggtaaagtcccgcaacgagcgcaacccttattgttagttgctacgcaagagcactctagcgagactgccgttgacaaaacggaggaaggtggggacgacgtcaaatcatcatgccccttacgtcctgggccacacacgtactacaatggcggccaacaaagagaggcaagaccgcgaggtggagaaaatctcaaaaagccgtcccagttcggatcgcaggctgcaacccgcctgcgtgaagttggaatcgctagtaatcgcggatcagcatgccgcggtgaatacgttcccgggccttgtacacaccgcccgtcacaccatgagagtcgggaacacccgaagtccgtagcctaaccgcaaggggggcgcggccgaaggtgggttcgataattggggtgaagtcgtaacaaggtagccgt

SEQ ID NO: 32 - DNA, 1489 bp, Blautia coccoides
  rRNA 1..1489: note = 16S rRNA coding gene sequence of Clostridium strain 12
  source 1..1489: mol_type = genomic DNA
agagtttgatcatggctcaggatgaacgctggcggcgtgcttaacacatgcaagtcgaacgggctcatattgaaacctagtgatgtatgagttagtggcggacgggtgagtaacgcgtggagaacctgccgtatactgggggataacacttagaaataggtgctaataccgcataagcgcacagcttcgcatgaagcagtgtgaaaaactccggtggtatacgatggatccgcgtctgattagctggttggcggggtaacagcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaatactgaccttacggtcagcagacggtacctgactaagaagccccggctaactacgtgccagcagccgcggtaatacgtagggggcaagcgttatccggatttactgggtgtaaagggagcgtagacggcagcgcaagtctgaagtgaaatcccatggcttaaccatggaactgctttggaaactgtgcagctggagtgcaggagaggtaagcggaattcctagtgtagcggtgaatgcgtagatattagaggacaccagtggcgatgcggcttactggactgtactgacgttgagctcgaaagcgtggggagcaccagaattagaatactgtagtcacgccgtaaccgatgatactaggtgtgggggaccaaggtctcgtgccggcggcaaacgcattaagtaatccacctggggagtacgttcgcaagaatgaaactcaaaggaattgacggggacccgcacaagcggtggagcatgtggtttaattcgaagcaacgcgaagaaccttacctggtcttgacatcccgatgacgagtgagcaaagtcactttcccttcggggcattggagacaggtggtgcatggttgtcgtcagctcgtgtcgtgagatgttgggttaagtcccgcaacgagcgcaacccctatttccagtagccagcaggtagagctgggcactctggagagactgcccgggataaccgggaggaaggcggggatgacgtcaaatcatcatgccccttatgatcagggctacacacgtgctacaatggcgtaaacaaagggaagcgagacggtgacgttaagcaaatcccaaaaataacgtcccagttcggattgtagtctgcaactcgactacatgaagctggaatcgctagtaatcgcgaatcagaatgtcgcggtgaatacgttcccgggtcttgtacacaccgcccgtcacaccatgggagtcggaaatgcccgaagtcagtgacctaaccgaaaggaaggagctgccgaaggtggagccggtaactggggtgaagtcgtaacaaggtagccgt

SEQ ID NO: 33 - DNA, 1456 bp, Blautia coccoides
  rRNA 1..1456: note = 16S rRNA coding gene sequence of Clostridium strain 13
  source 1..1456: mol_type = genomic DNA
agagtttgatcatggctcaggatgaacgctggcggcgtgcttaacacatgcaagtcgaacgaagcacttgagaacgattcttcggatgaggacttttgtgactgagtggcggacgggtgagtaacgcgtgggtaacctgccctatacagggggataacagttagaaatgactgctaataccgcataagcgcactaaaaccgcatggttcggtgtgaaaaactgaggtggtataggatggacccgcgtctgattagcttgttggtggggtaacggctcaccaaggcgacgatcagtagccggcctgagagggcgaccggccacattgggactgagacacggcccaaactcctacgggaggcagcagtgggggatattgcacaatggggggaaccctgatgcagcgacgccgcgtgggtgaagaagcgcctcggcgcgtaaagccctgtcagcagggaagaaaatgacggtacctgaagaagaagccccggctaactacgtgccagcagccgcggtaatacgtaggggcaagcgttattccgggatttactgggtgtaaagggggcgcagacggcgatgcaagccaggagtgaagcccggggcccacccgggactgctcttggactgcgtgctggagtgcagaaggggcagcgatcctgtgtaccgtgaattgcgtagatatcagagacacgttgcgagcgctgctgactgcactgacgtgagcgaagctggagcacagatagatactgtagtcagcgtaacgatgatactagtgtcgggagcagagactgcccgttgcggcagcccaacgcattagtattccacttggggagtacgtttcgcagaatgaacttcaaggaaatgacggggacccgcacaaggcggtggagcatgtggtttaattcgaagcaacgcgaagaaccttaccaggccttgacatcccccctggatggcccgtaacggggtcagcctttcggggcaggggagacaggtggtgcatggttgtcgtcagctcgtgtcgtgagatgttgggttaagtcccgcaacgagcgcaacccctgcccgcagtagccagcattttagatggggactctgcggggactgccggggacaacccggaggaaggcggggatgacgtcaaatcatcatgccccttatggcctgggctacacacgtgctacaatggcgccgacagagggaggcgaagcggcgacgcggagcgaaccccaaaaacggcgtcccagttcggattgtagtctgcaacccgactacatgaagccggaatcgctagtaatcgcggatcagaatgccgcggtgaatacgttcccgggtcttgtacacaccgcccgtcacaccatgggagccgggaatgcccgaagtctgtgaccgaacccgtaaggggaggggcagccgaaggcaggctcggtgactggggtgaagtcgtaacaaggtagccgt

SEQ ID NO: 34 - DNA, 1475 bp, Clostridium leptum
  rRNA 1..1475: note = 16S rRNA coding gene sequence of Clostridium strain 14
  source 1..1475: mol_type = genomic DNA
agagtttgatcctggctcaggacgaacgctggcggcgtgcttaacacatgcaagtcgaacggagcacccctgaaggagttttcggacaacggatgggaatgcttagtggcggactggtgagtaacgcgtgaggaacctgccttccagagggggacaacagttggaaacgactgctaataccgcatgatgcgttggagccgcatgactccgacgtcaaagatttatcgctggaagatggcctcgcgtctgattagctagttggtgaggtaacggcccaccaaggcgacgatcagtagccggactgagaggttggccggccacattgggactgagatacggcccagactcctacgggaggcagcagtggggaatattgggcaatggacgcaagtctgacccagcaacgccgcgtgaaggaagaaggctttcgggttgtaaacttcttttaagggggaagagcagaagacggtaccccttgaataagccacggctaactacgtgccagcagccgcggtaatacgtaggtggcaagcgttgtccggatttactgggtgtaaagggcgtgcagccggagagacaagtcagatgtgaaatccacgggctcaacccgtgaactgcatttgaaactgtttcccttgagtgtcggagaggtaatcggaatttccttgtgtagcggtgaatgcgtagatataaggaaggacacagtggcgagcggattactggacgatactgacgtgagcgcgaaagcgtgggggagcaacagaaattagatactgtagtgcagctgtaacgatcgatactagttgcgggactgaccccttgcgtgcgagttacacaataagtatcgcacctgggagtacgatcgcaaggttggaactcaaaggaattgacggggcccgcacaagcgttggattatgtggtttaattcgaagcaacgcgaagaaccttaccagggcttgacatcctgctaacgaagtagagatacattaggtgcccttcggggaaagtagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggcggggacgacgtcaaatcatcatgccccttatgtcctgggctacacacgtaatacaatggcggttaacaaagggatgcaaagccgcgaggcagagcgaaccccaaaaagccgtcccagttcggatcgcaggctgcaacccgcctgcgtgaagtcggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgt

SEQ ID NO: 35 - DNA, 1480 bp, Blautia coccoides
  rRNA 1..1480: note = 16S rRNA coding gene sequence of Clostridium strain 15
  source 1..1480: mol_type = genomic DNA
agagtttgatcctggctcaggatgaacgctggcggcgtgcttaacacatgcaagtcgaacgggctcatattgaaacctagtgatgtatgagttagtggcggacgggtgagtaacgcgtggagaacctgccgtatactgggggataacacttagaaataggtgctaataccgcataagcgcacagcttcgcatgaagcagtgtgaaaaactccggtggtatacgatggatccgcgtctgattagctggttggcggggtaacagcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaatactgaccttacggtcagcagacggtacctgactaagaagccccggctaactacgtgccagcagccgcggtaatacgtaggggcaagcgttatccggatttactgggtgtaaagggagcgtagacggcagcgcaagtctgaagtgaaatcccatggcttaacccatggaactgctttggaaactgtgcagctggagtgcaggagaggtaagcggaattcctagtgtagcgtgaaatgcgtagattattaggaggacaacagtgcgagcgctactgacgtgaggctcgaagcgtgggagcaaacaggattagatacctggtagtcacgcgtaaacgatgattactagggtgttgggggaccaaggtcttcggtgccggcgcaaacgcattaagtaatccacctggggagtacgttcgcaagaatgaaactcaaaggaattgacggggacccgcacaagcggtggagcatgtggtttaattcgaagcaacgcgaagaaccttacctggtcttgacatcccgatgacgagtgagcaaagtcactttcccttcggggcattggagacaggtggtgcatggttgtcgtcagctcgtgtcgtgagatgttgggttaagtcccgcaacgagcgcaacccctatttccagtagccagcaggtagagctgggcactctggagagactgcccgggataaccgggaggaaggcggggatgacgtcaaatcatcatgccccttatgatcagggctacacacgtgctacaatggcgtaaacaaagggaagcgagacggtgacgttaagcaaatcccaaaaataacgtcccagttcggattgtagtctgcaactcgactacatgaagctggaatcgctagtaatcgcgaatcagaatgtcgcggtgaatacgttcccgggtcttgtacacaccgcccgtcacaccatgggagtcggaaatgcccgaagtcagtgacctaaccgaaaggaaggagctgccgaaggtggagccggtaactggggtgaagtcgtaacaaggtagccgt

SEQ ID NO: 36 - DNA, 1486 bp, Clostridium papyrosolvens
  rRNA 1..1486: note = 16S rRNA coding gene sequence of Clostridium strain 16
  source 1..1486: mol_type = genomic DNA
agagtttgatcctggctcaggataaacgctggcggcgcacataagacatgcaagtcgaacggacttaactcattcttttagattgagagcggttagtggcggactggtgagtaacacgtaagcaacctgcctatcagaggggaataacagtgagaaatcattgctaataccgcatatgctcacagtatcacatgatacagtgaggaaaggagcaatccgctgatagatgggcttgcgcctgattagttagttggtggggtaacggcctaccaagacgacgatcagtagccggactgagaggttgaacggccacattgggactgagatacggcccagactcctacgggaggcagcagtcgggaatattgcgcaatggaggaaactctgacgcagtgacgccgcgtataggaagaaggttttcggattgtaaactattgtcgttagggaagataaaagactgtacctaaggaggaagccccggctaactatgtgccagcagccgcggtaatacatagggggcaagcgttatccggaattattgggtgtaaagggtgcgtagacggaagaacaagttggttgtgaaatccctcggctcaactgaggaactgcaaccaaaactattctccttgagtgtcggagaggaaagtggaattcctagtgtagcggtgaaatgcgtagatattaggaggaacaccagtggcgaaggcgactttctggacgataactgacgttgaggcacgaaagtgtggggagcaaacaggattagataccctggtagtccacactgtaaacgatggatactaggtgtagggtgtattaagcactctgtgccgccgctaacgcattaagtatcccacctggggagtacgaccgcaaggttgaaactcaaaggaattgacgggggcccgcacaagcagtggagtatgtggtttaattcgaagcaacgcgaagaaccttaccagggcttgacatataccggaatatactagagatagtatagtccttcgggactggtatacaggtggtgcatggttgtcgtcagctcgtgtcgtgagatgttgggttaagtcccgcaacgagcgcaacccctatcgttagttgctagcaggtaatgctgagaactctagcgagactgccggtgataaatcggaggaaggtggggatgacgtcaaatcatcatgccctttatgtcctgggctacacacgtactacaatggccgtaacagagggaagcaatatagtgatatggagcaaaaccctaaaagcggtctcagttcggattgaaggctgaaattcgccttcatgaagccggaattgctagtaatggcaggtcagcatactgccgtgaatacgttcccgggccttgtacacaccgcccgtcacaccatgagagttggaaatacccgaagcctgtgagctaactgtaaagaggcagcagtcgaaggtagagccaatgattggggtgaagtcgtaacaaggtagccgt

SEQ ID NO: 37 - DNA, 1493 bp, Clostridium leptum
  rRNA 1..1493: note = 16S rRNA coding gene sequence of Clostridium strain 17
  source 1..1493: mol_type = genomic DNA
agagtttgatcatggctcaggatgaacgctggcggcgtgcttaacacatgcaagtcgaacgagaaccaacggattgaggattcgtccaaatgaagttggggaaagtggcggacgggtgagtaacgcgtgagcaatctgccttggagtggggaataacggttggaaacagccgctaataccgcatgatgcgtctgggaggcatctctctggacgccaaagatttatcgctctgagatgagctcgcgtctgattagctagttggcggggcaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttaagggggacgaacaaatgacggtaccccttgaataagccacggctaactacgtgccagcagccgcggtaatacgtaggtggcaagcgttatccggatttattgggtgtaaagggcgtgtaggcgggaatgcaagtcagatgtgaaaactatgggctcaacccatagcctgcatttgaaactgtatttcttgagtgctggagaggcaatcggaattccgtgtgtagcggtgaaatgcgtagatatacggaggaacaccagtggcgaagcggattgctggacagtaactgacgctgaggcgcgaaagcgtggggagcaaacagggattagataccctggtagtcacgccgtaaacgatggatactaggtgtggggggactgaccccctccgtgccgcagctaacgcaataagtatcccacctggggagtacgatcgcaagggttgaaactcaaaggaattgacgggggcccgcacaagcggtggagtatgtggtttaattcgaagcaacgcgaagaaccttaccagggcttgacatcctgctaacgaaccagagatggatcaggtgcccttcggggaaagcagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttacgtcctgggccacacacgtactacaatggcggccaacaaagagaggcaagaccgcgaggtggagcaaatctcaaaaagccgtcccagttcggatcgcaggctgcaacccgcctgcgtgaagttggaatcgctagtaatcgcggatcagcatgccgcggtgaatacgttcccgggccttgtacacaccgcccgtcacaccatgagagtcgggaacacccgaagtccgtagcctgaccgcaaggggggcgcggccgaaggtgggttcgataattggggtgaagtagtaacaaggtagccgt

SEQ ID NO: 38 - DNA, 1493 bp, Clostridium leptum
  rRNA 1..1493: note = 16S rRNA coding gene sequence of Clostridium strain 18
  source 1..1493: mol_type = genomic DNA
agagtttgatcctggctcaggacgaacgctggcggcgcgcctaacacatgcaagtcgaacggagcttatatttcagaagttttcggatggacgagagataagcttagtggcggacgggtgagtaacacgtgagcaacctgcctttcagagggggataacagttggaaacgactgctaataccgcataacgctgcgatggggcatcccgatgcagccaaaggagcaatccgctgaaagatgggctcgcggccgattagctagttggtggggcaacggcccaccaaggcgacgatcggtagccggactgagaggttgatcggccacattgggactgagacacggcccagactcctacgggaggcagcagtgggggatattgcacaatggaggaaactctgatgcagcgacgccgcgtgagggaagacggtcttcggattgtaaacctctgtctttggggaagaaaatgacggtacccaaagaggaagctccggctaactacgtgccagcagccgcggtaatacgtacggagcgagcgttgtccggaattactgggtgtaaagggagcgtacgcgggcgagaaagttgaatgttaaatctaccggcttaactggtagctgcgttcaaaacttcttgtcttgagtgaagtagaggcaggcggaattcctagtgtagcggtgaaatgcgtagatataggaggacaccagtgggcgaagccgcctgctgggctttaactgacgctgaggctcgaaagcgtggggagcaaaccaggattagataccctggtagtcaacgctgtaaacgatgattactaggtgtgggggggactgaccccctccgtgccgcagttaacacaataagtatccacctggggagtacggccgcaaagtttgaaaactcaaaaggaatgacgggggcccgcacaaagcagtggagtatgtggtttaatttcgaagcaacgcgaagaaccttaccaggtcttgacatcgtgcgcatagcctagagataggtgaagcccttcggggcgcacagacaggtggtgcatggttgtcgtcagctcgtgtcgtgagatgttgggttaagtcccgcaacgagcgcaacccttattattagttgctacgcaagagcactctaatgagactgccgttgacaaaacggaggaaggtggggatgacgtcaaatcatcatgccccttatgacctgggctacacacgtactacaatggcactgaaacagagggaagcgacatcgcgaggtgaagcgaatcccaaaaaagtgtcccagttcggattgcaggctgcaactcgcctgcatgaagtcggaattgctagtaatcgcggatcagcatgccgcggtgaatacgttcccgggccttgtacacaccgcccgtcacaccatgggagtcggtaacacccgaagccagtagcctaaccgcaaggagggcgctgtcgaaggtgggattgatgactggggtgaagtcgtaacaaggtagccgt

SEQ ID NO: 39 - DNA, 1483 bp, Clostridium leptum
  misc_feature 1..1483: note = 16S rRNA coding gene sequence of Clostridium strain 19
  source 1..1483: mol_type = genomic DNA
agagtttgatcctggctcaggacgaacgctggcggcacgcctaacacatgcaagtcgaacggagtgaagatgcttgcatctgaacttagtggcggacgggtgagtaacacgtgagcaacctgcctttcagagggggataacgtttggaaacgaacgctaataccgcataaaatatcggagtcgcatggcactgatatcaaaggagcaatccgctgaaagatgggctcgcgtccgattaggcagttggcggggtaacggcccaccaaaccgacaatcggtagccggactgagaggttgaacggccacattgggactgagacacggcccagactcctacgggaggcagcagtgggggatattgcacaatgggggaaaccctgatgcagcgatgccgcgtgaatgaagacggccttcgggttgtaaagttctgtcgcaggggacgaaaatgacggtaccctgcaagaaagctccggctaactacgtgccagcagccgcggtaatacgtagggagcaagcgttgtccggaattactgggtgtaaagggagcgtaggcgggaggataaagttgaatgtgaaatctatgggctcaacccatagctgcgttcaaaactgttcttcttgagtgaagtagaggcaggcggaattcctagtgtagcggtgaaatgcgtagatattaggaggaacaccagtggcgaaagcggcctgctgggcttttactgacgctgaggctcgaaagcgtgggtagcaaacagaattagattaccctgttattcacggcggtaaacgatgattactaggtttgggttgacctgacccccattcgtgccggaagtaacaccataaagtaatccacctgggggagtacggccgccaggttgaaacttcaaaaggaattgacgggggcccgcacaagcagtggaggtatgtggtttaatttcgacgcaaacgcgaagaaccttaccagggtcttgacatcgagtgacggacatagagatatgtctttcctttcgggacacgaagacaggtggtgcatggttgtcgtcagctcgtgtcgtgagatgttgggttaagtcccgcaacgagcgcaacccttaccattagttgctacgcaagagcactctgatgggactgccgttgacaaaacggaggaaggtggggatgacgtcaaatcatcatgccccttatgacctgggcgacacacgtactacaatggcggtcaacagagggaggcaaagccgcgaggcagagcaaacccctaaaagccgtctcagttcggattgcaggctgcaactcgcctgcatgaagtcggaattgctagtaatcgcggatcagcatgccgcggtgaatacgttcccgggccttgtacacaccgcccgtcacaccatgagagccggtaacacccgaagtcaatagtctaaccgcaaggaggacattgccgaaggtgggattggtaattggggtgaagtcgtaacaaggtagccgt

SEQ ID NO: 40 - DNA, 1511 bp, Blautia coccoides
  rRNA 1..1511: note = 16S rRNA coding gene sequence of Clostridium strain 20
  source 1..1511: mol_type = genomic DNA
agagtttgatcctggctcaggatgaacgctggcggcgtgcttaacacatgcaagtcgaacgggctcatattgaaacctagtgatgtatgagttagtggcggacgggtgagtaacgcgtggagaacctgccgtatactgggggataacacttagaaataggtgctaataccgcataagcgcacagcttcgcatgaaacagtgtgaaaaactccggtggtatacgatggatccgcgtctgattagctggttggcggggtaacagcccaccaaggcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaatactgaccttacggtcagcagacggtacctgactaagaagccccggctaactacgtgccagcagccgcggtaatacgtaggggcaagcgttatccggatttactgggtgtaaagggagcgtagacggcagcgcaagtctgagtgaaatcccatggcttaaccatggaactgctttggaaactgtgcagctggagtgcaggagaggtaaagcggaattcctagtgtagcgggtgaaatgcgtagatataggaggaacaacagtggcggaaggcggctactgggactgtaactgacgttgaggctcgaaagcgtggggagcaaacaggattagataccctggtagtcacgccgtaaacgatgattactaggtgttgggggaccataggtcttcggtgccggcgcaaacgcaattaagtaatccacctgggggagtacgttcgcaagaatgaaactcaaaggaattgacggggacccgcacaaagcggtggagcatgtggtttaattcgaaagcaaacgcgaagaaaccttacctggtcttgacatcccgatgacgagtgagcaaagtcactttcccttcggggcaattggagacaggtggtgcatgggttgtcgtcagctcgtgtcgtgagatgttgggttaagtcccgcaacgagcgcaacccctatttccagtagccagcaggtagagctgggcactctggagagactgcccgggataaccgggaggaaggcggggatgacgtcaaatcatcatgccccttatgatcagggctacacacgtgctacaatggcgtaaacaaagggaagcgagacggtgacgttaagcaaatcccaaaaataacgtcccagttcggattgtagtctgcaactcgattacatgaagctggaatcgctagtaatcgcgaatcagaatgtcgcggtgaatacgttcccgggtcttgtacacaccgcccgtcacaccatgggagtcggaaatgcccgaagtcagtgacctaaccgaaaggaaggagctgccgaaggtggagccggtaactggggtgaagtagataacaaggtagccgt

SEQ ID NO: 41 - DNA, 1495 bp, Clostridium leptum
  rRNA 1..1495: note = 16S rRNA coding gene sequence of Clostridium strain 21
  source 1..1495: mol_type = genomic DNA
agagtttgatcctgcgctcaggacgaacgctggcggcgcgcctaacacatgcaagtcgaacgggactattttgggagaagttttcggatggatctcgggatagtttagtggcggacgggtgagtaacgcgtgggcaacctgccttacacagggggataacaattagaaatgattgctaataccgcataagaccacggtactgcatggtacagtggtaaaaactgaggtggtgtaagatgggcccgcgtctgattaggtagttggtggggtagaagcctaccaagccgacgatcagtagccgacctgagagggcgaccggccacattgggactgagacacggcccaaactcctacgggaggcagcagtggggaatattgcacaatgggggaaaccctgatgcagcgacgccgcgtgagtgaggaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggacttgcaagtctgatgtgaaaatccgggggcccaaccccggaactgcattggaaactgtatatctagagtgtcggagaggcaagtggaatttcctggtgtagcggtgaaatgcgtagatatcagaggaacaccagtggcgaaggcgcttgcctgacgatgactgacgttgaagctcgaaaagcgtgggtagcaaacagaattagataccctggtaagtcaacccggtaaacgatgattactaggttttggttggactgaccccatccgtgccggagtaacaccaataagttatccaacctgggaagtacggccggcaggttgaaactcaaaaggaaatgacgggggcccgcacaagcagttgaagtatgtgggttaattcgacgcaaacgcgaagaaccttaccaggtcttgacatcgagtgacggacatagagatatgtctttccttcgggacacgaagacaggtggtgcatggttgtcgtcagctcgtgtcgtgagatgttgggttaagtcccgcaacgagcgcaacccttaccattagttgctacgcaagagcactctgatgggactgccgttgacaaaacggaggaaggtggggatgacgtcaaatcatcatgccccttatgacctgggcgacacacgtactacaatggcggtcaacagagggaggcaaagccgcgaggcagagcaaacccctaaaagccgtctcagttcggattgcaggctgcaactcgcctgcatgaagtcggaattgctagtaatcgcggatcagcatgccgcggtgaatacgttcccgggccttgtacacaccgcccgtcacaccatgagagccggtaacacccgaagtcaatagtctaaccgcaaggaggacattgccgaaggtgggattggtaattggggtgaagtcgtaacaaggtagccgt

SEQ ID NO: 42 - DNA, 1491 bp, Clostridium leptum
  rRNA 1..1491: note = 16S rRNA coding gene sequence of Clostridium strain 22
  source 1..1491: mol_type = genomic DNA
agagtttgatcctggctcaggatgaacgctggcggcgtgcttaacacatgcaagtcgaacgagaatccagtgaaggagttttcggacaacggatctggaggaaagtggcggacgggtgagtaacgcgtgagcaatctgccttggagtggggaataacggttggaaacagccgctaataccgcatgatgcgtctgggaggcatctctctggacgccaaagatttatcgctctgagatgagctcgcgtctgattagcttgttggcggggtaaaggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttctgagggacgaagaaagtgacggtacctcaggaataagccacggctaactacgtgccagcagccgcggtaatacgtaggtggcaagcgttatccggatttattgggtgtaaagggcgtgtaggcgggaaagcaagtcagatgtgaaaactcagggctcaaccctgagcctgcatttgaaactgtttttcttgagtgctggagaggcaatcggaattccgtgtgtagcggtgaaatgcgtagatatacggaggacaccagtggcgaagcggattgctggacagtaactgacgctgaggcgcgaagcgtggggagcaaacaggattagataccctggtagtccacgccgtaaacgatggatactaggtgtggggggactgaccccctccgtgccgcagctaacgcaataagtatcccacctggggagtacgatcgcaaggttgaaactcaaaggaattgacgggggcccgcacaagcggtggagtatgtggtttaattcgaagcaacgcgaagaaccttaccagggcttgacatcctgctaacgaaccagagatggattaggtgcccttcggggaaagcagagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttacgtcctgggccacacacgtactacaatggcggccaacaaagagaggcaagaccgcgaggtggagcaaatctcaaaaagccgtcccagttcggatcgcaggctgcaacccgcctgcgtgaagttggaatcgctagtaatcgcggatcagcatgccgcggtgaatacgttcccgggccttgtacacaccgcccgtcacaccatgagagtcgggaacacccgaagtccgtagcctaaccgcaaggggggcgcggccgaaggtgggttcgataattggggtgaagtcgtaacaaggtagccgt

SEQ ID NO: 43 - DNA, 1495 bp, Clostridium leptum
  rRNA 1..1495: note = 16S rRNA coding gene sequence of Clostridium strain 23
  source 1..1495: mol_type = genomic DNA
agagtttgatcctgtgcctcaggatgaacgctggcggcgtgcttaacacatgcaagtcgaacgagaaccaacggattgaggattcgtccaaatgaagttggggaaagtggcggacgggtgagtaacgcgtgagcaatctgccttggagtggggaataacggttggaaacagccgctaataccgcatgatgcgtctgggaggcatctctctggacgccaaagatttatcgctctgagatgagctcgcgtctgattagctagttggcggggcaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttaagggggacgaacaaatgacggtaccccttgaataagccacggctaactacgtgccagcagccgcggtaatacgtaggtggcaagcgttatccggatttattgggtgtaaagggcgtgtaggcgggaatgcaagtcagatgtgaaaactatgggctcaacccatagcctgcatttgaaactgtatttcttgagtgctggagaggcaatcggaattccgtgtgtagcggtgaaatgcgtagatatacggaggaacaccagtggcgaaggcggattgctggacagtaactgacgctgaggcgcgaaagcgtggggagcaaacaggattagataccctggtagtccacgccgtaaacgatggatactaagtgtggggggactgaccccctccgtgccgcagctaacgcaataagtatcccacctggggagtacgatcgcaaggttgaaactcaaaggaattgacgggggcccgcacaagcggtggagtatgtggtttaattcgaagcaacgcgaagaaccttaccagggcttgacatcctgctaacgaaccagagatggatcaggtgcccttcggggaaagcagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttacgtcctgggccacacacgtactacaatggcggccaacaaagagaggcaagaccgcgaggtggagcaaatctcaaaaagccgtcccagttcggatcgcaggctgcaacccgcctgcgtgaagttggaatcgctagtaatcgcggatcagcatgccgcggtgaatacgttcccgggccttgtacacaccgcccgtcacaccatgagagtcgggaacacccgaagtccgtagcctgaccgcaaggggggcgcggccgaaggtgggttcgataattggggtgaagtcgtaacaaggtagccgt

SEQ ID NO: 44 - DNA, 1440 bp, Clostridium leptum
  rRNA 1..1440: note = 16S rRNA coding gene sequence of Clostridium strain 24
  source 1..1440: mol_type = genomic DNA
agagtttgatcctggctcaggacgaacgctggcggcacgcctaacacatgcaagtcgaacggagtgaagatgctcgcatctgaacttagtggcggacgggtgagtaacacgtgagcaacctgcctttcagagggggataacgtttggaaacgaacgctaataccgcataaaatatcggagtcgcatggcactgatatcaaaggagtaatccgctgaaagatgggctcgcgtccgattaggcagttggcggggtaacggcccaccaaaccgacaatcggtagccggactgagaggttgaacggccacattgggactgagacacggcccagactcctacgggaggcagcagtgggggatattgcacaatgggggaaaccctgatgcagcgatgccgcgtgaatgaagacggccttcgggttgtaaagttctgtcgcaggggacgaaaatgacggtaccctgcaagaaagctccggctaactacgtgccagcagccgcggtaatacgtagggagcaagcgttgtccggaattactgggtgtaaagggagcgtaggcgggaggataagttgaatgtgaaatctatgggctcaacccatagttgcgttcaaaactgttcttcttgagtgaagtagaggcaggcggaattcctagtgtagcggtgaaatgcgtagatattagaggaacaccagtggcgaagcggcctgctgggcttttactgacgctgagctcgaaagcgtgggtagcaacaggattagataccctggtagtccacgcggtaaacgatgattactagtgtgggtggactgacccatccatgccggagttaacacaatagtaatccacctggggagtacgcgcagtgaactcaaaggattgacggggcccgcacaagcagtgagtatgtggtttattcgacgcacgcgagactacagtcttgacatcgatgacggactagagatatgtctttctcggacacgaagacaggtggtgcatggttgtcgtcagctcgtgtcgtgagatgttgggttaagtcccgcaacgagcgcaacccttaccattagttgttacgcaagagcactctaatgggactgccgttgacaaaacggaggaaggtggggatgacgtcaaatcatcatgccccttatgacctgggcgacacacgtactacaatggcggtcaacagagggaggcaaagccgcgaggcagagcaaacccctaaaagccgtctcagttcggattgcaggctgcaactcgcctgcatgaagtcggaattgctagtaatcgcggatcagcatgccgcggtgaatacgttcccgggccttgtacacaccgcccgtcacaccatgagagccggtaacacccgaagtcaatagtctaaccgcaaggaggacattgccgaaggtgggatggtaattggggtgaagtagtaacaaggtagccgt

SEQ ID NO: 45 - DNA, 1495 bp, Clostridium leptum
  rRNA 1..1495: note = 16S rRNA coding gene sequence of Clostridium strain 25
  source 1..1495: mol_type = genomic DNA
agagtttgatcctggctcaggatgaacgctggcggcgtgcttaacacatgcaagtcgaacgagaaccattggatcgaggattcgtccaagtgaaggtggggaaagtggcggacgggtgagtaacgcgtgagcaatctgccttggagtggggaataacggctggaaacagccgctaataccgcatgatacagctgggaggcatctccctggctgtcaaagatttatcgctctgagatgagctcgcgtctgattagctagttggcggggtaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttgtcagggacgaagcaagtgacggtacctgacgaataagccacggctaactacgtgccagcagccgcggtaatacgtagtggcaagcgttatccggatttattggggtgtaaagggcgtgtaggcgggaatgcaagtcagatgtgaaaactatggggctcaacccatagcctgcatttgaaactgtatttcttgagtgctggagaggcaatcgaattccgtgtgtagcgggtgaaatgcgtagatatacggaggaacaccagtggcgaagcggattgctggacaagtaactgacgctgaggcgcgaaagcgtggggagcaaacaggattagataccctggtagtccacgccgtaaacgatggatactaggtgtggggggactgaccccctccgtgccgcagctaacgcaataagtatcccacctggggagtacgatcgcaaggttgaaactcaaaggaattgacgggggcccgcacaagcggtggagtatgtggtttaattcgacgcaacgcgaagaaccttaccagggcttgacatcctactaacgaaccagagatggattaggtgcccttcggggaaagtagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttacgtcctgggccacacacgtactacaatggcggccaacaaagagaggcaaagccgcgaggtggagcaaatctcaaaaagccgtcccagttcggatcgcaggctgcaacccgcctgcgtgaagttggaatcgctagtaatcgcggatcagcatgccgcggtgaatacgttcccgggccttgtacacaccgcccgtcacaccatgagagtcgggaacacccgaagtccgtagcctaaccgcaaggggggcgcggccgaaggtgggttcgataattggggtgaagtcgtaacaaggtagccgt

SEQ ID NO: 46 - DNA, 1495 bp, Clostridium leptum
  rRNA 1..1495: note = 16S rRNA coding gene sequence of Clostridium strain 26
  source 1..1495: mol_type = genomic DNA
agagtttgatcatggctcaggacgaacgctggcggcgtgcttaacacatgcaagtcgaacggagcacccctgaaggagttttcggacaacggatgggaatgcttagtggcggactggtgagtaacgcgtgaggaacctgccttccagagggggacaacagttggaaacgactgctaataccgcatgatgcgttggagccgcatgactccgacgtcaaagatttatcgctggaagatggcctcgcgtctgattagctagttggtgaggtaacggcccaccaaggcgacgatcagtagccggactgagaggttggccggccacattgggactgagatacggcccagactcctacgggaggcagcagtggggaatattgggcaatggacgcaagtctgacccagcaacgccgcgtgaaggaagaaggctttcgggttgtaaacttcttttaagggggaagagcagaagacggtaccccttgaataagccacggctaactacgtgccagcagccgcggtaatacgtaggtggcaagcgttgtccggatttactgggtgtaaagggcgtgcagccggagagacaagtcagatgtgaaatccacgggctcaacccgtgaactgcatttgaaactgtttcccttgagtgtcggagagggtaatcggaattcctttgtgtagcggtgaaatgcgtagatataagaagaacaccagtggcgaaggcggattactggacgataactgacggtgaggcgcgaaagcgtgggggagcaacagattaaataccctggtagtccacgctgttaacgatcgatactaggtgtgccgggactgaccccctgcgtgcccggagttaaccacaataagtatcgcacctggggagtacgatcgcaaggtgaacttcaaaggaattgacgggggcccgccccaagccgtggattatgtggttaattcgaagcaacgcgaagaacctacccagggcttgacatcctgctaacgaagtagagatacattaggtgccctttcggggaaagcagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggcggggacgacgtcaaatcatcatgccccttatgtcctgggctacacacgtaatacaatggcggttaacaaagggatgcaaagccgcgaggcagagcgaaccccaaaaagccgtcccagttcggatcgcaggctgcaacccgcctgcgtgaagtcggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgt

SEQ ID NO: 47 - DNA, 1509 bp, Clostridium leptum
  rRNA 1..1509: note = 16S rRNA coding gene sequence of Clostridium strain 27
  source 1..1509: mol_type = genomic DNA
agagtttgatcctggctcaggatgaacgctggcggcgtgcttaacacatgcaagtcgaacggagtacccctgaaggagttttcggacaactgatgggactacttagtggcggacgggtgagtaacgcgtgagtaacctgccttggagtggggaataacagctggaaacagctgctaataccgcataatatgtctgtgtcgcatggcactggacatcaaagatttatcgctctgagatggactcgcgtctgattagctagttggcggggtaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttaagggggaagagcagaagacggtaccccttgaataagccacggctaactacgtgccagcagccgcggtaatacgtaggtggcaagcgttgtccggatttactgggtgtaaagggcgtgcagccggagagacaagtcagatgtgaaatccacgggctcaacccgtgaactgcatttgaaactgtttcctggagttcggagggtatggaattcttgttagcggtgaaatgctgtagatatgggagaaccaccagtgcgagggggcttccgggactgtacttgactgtagaggtctcaaagctgggggagcaccgaggaatgagataccgtgatagtcccacgcggtaacggatgattactaggtgttggggggacccaggctctttcggtgccgggcgcaaaccctttaggaattccacctggggaattacgtttggcaagaaaggaacttcaaagaaattgaacgggggacccccccaaccggtggaggcatggtgttttatttcggaggaacgggaagaacctttaccttgttctgaccttccggatgacgaagtgagcaaagtcaacttcccttcggggccatggaggacaggtggtggcatggttggtcgtcagctcgtgtcgtgagatgttgggttaagtcccgcaacgagcgcaacccctatttccagtagccagcaggtagagctgggcactctggagagactgcccgggataaccgggaggaaggcggggatgacgtcaaatcatcatgccccttatgatcagggctacacacgtgctacaatggcgtaaacaaagggaagcgagacggtgacgttaagcaaatcccaaaaataacgtcccagttcggattgtagtctgcaactcgactacatgaagctggaatcgctagtaatcgcgaatcagaatgtcgcggtgaatacgttcccgggtcttgtacacaccgcccgtcacaccatgggagtcggaaatgcccgaagtcagtgacctaaccgaaaggaaggagctgccgaaggtggagccggtaactggggtgaagtcgtaacaaggtagccgt

SEQ ID NO: 48 - DNA, 1583 bp, Clostridium leptum
  rRNA 1..1583: note = 16S rRNA coding gene sequence of Clostridium strain 28
  source 1..1583: mol_type = genomic DNA
agagtttgatcctggctcaggacgaacgctggcggcgcgcctaacacatgcaagtcgaacggagcttatatttcagaagttttcggatggacgagagataagcttagtggcggacgggtgagtaacacgtgagcaacctgcctttcagagggggataacagttggaaacgactgctaataccgcataacgctgcgatggggcatcccgatgcagccaaaggagcaatccgctgaaagatgggctcgcggccgattagctagttggtggggcaacggcccaccaaggcgacgatcggtagccggactgagaggttgatcggccacattgggactgagacacggcccagactcctacgggaggcagcagtgggggatattgcacaatggaggaaactctgatgcagcgacgccgcgtgagggaagacggtcttcggattgtaaacctctgtctttggggaagaaaatgacggtacccaaagaggaagctccggctaactacgtgccagcagccgcggtaatacgtaggggagcgagcgttgtccggaattactgggtgtaaagggagcgtagcgggcgagaaagttgaatgttaaatctaccggcttaactggtagctgcgtccaaaacttcttggtcttgagtgaaagtaagaggccagggcggaaattcttagtgtaagcgggtgaaaatgcgttagatattagggaggaaccaccaggtgggcgaagggcggcttgctgggctttaactggacggctggaggcttggaaaaggcgtggggagagcaaacacagggaattaagtataccctggtatatgtcacacgcttgtaaagagtatgattaacttagggtggtggggggaacttgaccctttcgtgtgcgcgcaggttaacacacatttagagtatatccaacttggggagagtacggccggcaaagtttgaaacttcaaaagggaaattgagaccggggggcccggccaccaagcacagtggagagtatggtgggtttaatttcgagaagcaaccggcggaagagaaactttaccagtccttgacatcggtggcggcataagccctagagattaggtgaagcccttcgggggccccaccagacaggtggtgcatggttgtcgtcagctcgtgtcgtgagatgttgggttaagtccccgcaaacgagcgcaacccttattattagtttgctacgcaagagcactctaatgagactgccgttgacaaaacggaggaaggtggggatgacgtcaaatcatcatgccccttatgacctgggctacacacgtactacaatggcactgaaacagagggaagcgacatcgcgaggtgaagcgaatcccaaaaaagtgtcccagttcggattgcaggctgcaactcgcctgcatgaagtcggaattgctagtaatcgcggatcagcatgccgcggtgaatacgttcccgggccttgtacacaccgcccgtcacaccatgggagtcggtaacacccgaagccagtagcctaaccgcaaggagggcgctgtcgaaggtgggattgatgactggggtgaagtcgtaacaaggtagccgt

SEQ ID NO: 49 - DNA, 1519 bp, Blautia coccoides
  rRNA 1..1519: note = 16S rRNA coding gene sequence of Clostridium strain 29
  source 1..1519: mol_type = genomic DNA
agagtttgatcatggctcaggatgaacgctggcggcgtgcctaacacatgcaagtcgaacgggtgtacgggaaggaaggcttcggccggaaaacctgtgcatgagtggcggacgggtgagtaacgcgtgggcaacctggcctgtacagggggataacacttagaaataggtgctaataccgcataacgggggaagccgcatggcttttccctgaaaactccggtggtacaggatgggcccgcgtctgattagccagttggcagggtaacggcctaccaaagcgacgatcagtagccggcctgagagggcggacggccacactgggactgagacacggcccagactcctacgggaggcagcagtgggggatattgcacaatggggggaaccctgatgcagcgacgccgcgtgggtgaagaagcgcctcggcgcgtaaagccctgtcagcagggaaagaaaatgacggtacctgaagaagaagccccgggctaactacgtgccagcagccggcggtaattacgtaggggggcaggcgttatccggatttactgggtggtaaaggggggcgcaaacggcgatggcaggccaggaatggaaagcccgggggcccaaccccgggacttgctcttgggaaactggcttggctgggagtggcaggaggggcaggcggaaattcctggtggtagcggtggaaaatggctaaaaatcaagaagaaaaaccggtggggaaaggcggcctggtgggactgcgaactgacgttgaaggcccgaaagcgtggggaacaaacaggatagattccctggtagttccacgccgtaaacgatgattactaggtgtcggggagcagagactgcccggtgccgcagccaacgcattaagtaatccacctggggagtacgttcgcaagaatgaaactcaaaggaattgacggggacccgcacaagcggtggagcatgtggtttaattcgaagcaacgcgaagaaccttacctggtcttgacatcccgatgacgagtgagcaaagtcactttcccttcggggcattggagacaggtggtgcatggttgtcgtcagctcgtgtcgtgagatgttgggttaagtcccgcaacgagcgcaacccctatttccagtagccagcaggtagagctgggcactctggagagactgcccgggataaccgggaggaaggcggggatgacgtcaaatcatcatgccccttatgatcagggctacacacgtgctacaatggcgtaaacaaagggaagcgagacggtgacgttaagcaaatcccaaaaataacgtcccagttcggattgtagtctgcaactcgactacatgaagctggaatcgctagtaatcgcgaatcagaatgtcgcggtgaatacgttcccgggtcttgtacacaccgcccgtcacaccatgggagtcggaaatgcccgaagtcagtgacctaaccgaaaggaaggagctgccgaaggtggagccggtaactggggtgaagtcgtaacaaggtagccgt

SEQ ID NO: 50 - DNA, 1497 bp, Blautia coccoides
  rRNA 1..1497: note = 16S rRNA coding gene sequence of Clostridium strain 30
  source 1..1497: mol_type = genomic DNA
agagtttgatcctggctcaggatgaacgctggcggcgtgcctaacacatgcaagtcgaacggggtatataagcggaagtttacggatggaaggttatatacttagtggcggacgggtgagtaacgcgtgggcaacctgccccgtgccgggggataccgcctggaaacaggcgctaataccgcataagcgcatacagccgcatgggtgtatgcggaaagctccggcggcacgggatgggcccgcgcccgattagccagttggcggggtaacggcccaccaaagcgacgatcggtagccggcctgagagggcggacggccacattgggactgagacacggcccaaactcctacgggaggcagcagtggggaatattgcacaatgggggaaaccctgatgcagcaacgccgcgtgggtgaaggagcgtttcggcgcgtaaagccctgtcagcggggaagaagaaagacggtacccgaccaagaagccccggctaactacgtgccagcagccgcggtaatacgtagggggcgagcgttatccggaattactgggtgtaaagggagcgtagacggcgaggtaagcctgaagtggaagcccgcggcccaaccgcggaactgctttgggaactgttttgctggagtatgggaggggtaagcggaattcctggtgtagcggtgaaatgcgtagatatcaggaggaacaccggtggcgaaggcggcttactggaccataactgacgttgaggctcgaaagcgtggggagcgaacaggattagataccctggtagtccacgcgtaaacgatgattaccaggtgtcgggtgtcgaaggacggcccggtgccgcagcgaacgcagtaagtaatccacctggggagtacgttcgcaagaatgaaactcaaaggaattgacggggacccgcacaagcggtggagcatgtggtttaattcgaagcaacgcgaagaaccttacccggccttgacatcccctggacagcatatgtaatgtatgtttccttcgggaccagggagacaggtggtgcatggttgtcgtcagctcgtgtcgtgagatgttgggtcaagtcccgcaacgagcgcaacccctgcccccagtagccagcatttaagatgggcactctggggggactgccggggataacccggaggaaggcggggatgacgtcaaatcatcatgccccttatggccggggctacacacgtgctacaatggcgtaaacagagggaggcgagacagcgatgttaagcgaaccccaaaaataacgtcccagttcggattgcagcctgcaactcggctgcatgaagctggaatcgctagtaatcgcggatcagaatgccgcggtgaatacgttcccgggtcttgtacacaccgcccgtcacaccatgggagtcgggaacgcccgaagccggtgaccgaacccgaaaggggaggagccgtcgaaggcgggcctggtgactggggtgaagtcgtaacaaggtagccgt

SEQ ID NO: 51 - DNA, 1475 bp, Clostridium leptum
  rRNA 1..1475: note = 16S rRNA coding gene sequence of Clostridium strain 31
  source 1..1475: mol_type = genomic DNA
gagtttgatcctggctcaggataaacgctggcggcgcacataagacatgcaagtcgaacgaacttaataccttgcttgcaaggtaagcggttagtggcggactggtgagtaacacgtaagaaatctgcctatcagaggggaataacagtgagaaatcactgctaataccgcatatgccatagttatcgcatgataatagtgggaaagaagcaattcgctgatagatgagcttgcggctgattagctagttggtggggtaacggcctaccaaggcgacgatcagtagccggcctgagagggtgaacggccacattgggactgagacacggcccaaactcctacgggaggcagcagtggggaatattgcacaatgggggaaaccctgatgcagcgacgccgcgtgagtgaagaagtatttcggtatgtaaagctctatcagcagggaagaaaatgacggtacctgactaagaaagccccggctaactacgtgccagcagccgcggtaatacgtagggggcaagcgttatccggatttactggtgtaaagggagcgtagacggcagcgcaagtctgagtgaaatcccatggcttacccatgaaactgctttggaaactgtgcagctggagtgcaggagaggtaagcggaatcctagtgtagcggttgaaatgcgtagattatcagaaggaacaccggtggccgaggcggcctgctgggcttttactgacgctgaggctcgaagcgtgggtagcaaacaggattagataccctggtagtccacgcggtaaacgatgattactaggtgtgggtggactgaccccatccgtgccggagttaacacaataagtaatccacctggggagtacggccgcaaggttgaaactcaaaggaattgacgggggcccgcacaagcagtggagtatgtggtttaattcgacgcaacgcgaagaaccttaccaggtcttgacatcgagtgacggacatagagatatgtctttccttcgggacacgaagacaggtggtgcatggttgtcgtcagctcgtgtcgtgagatgttgggttaagtcccgcaacgagcgcaacccttaccattagttgctacgcaagagcactctaatgggactgccgttgacaaaacggaggaaggtggggatgacgtcaaatcatcatgccccttatgacctgggcgacacacgtactacaatggcggtcaacagagggaggcaaagccgcgaggcagagcaaacccctaaaagccgtctcagttcggattgcaggctgcaactcgcctgcatgaagtcggaattgctagtaatcgcggatcagcatgccgcggtgaatacgttcccgggccttgtacacaccgcccgtcacaccatgagagccggtaacacccgaagtcaatagtctaaccgcaaggaggacattgccgaaggtgggattggtaattggggtgaagtcgtaacaaggtagccgt

SEQ ID NO: 52 - DNA, 1491 bp, Clostridium leptum
  rRNA 1..1491: note = 16S rRNA coding gene sequence of Clostridium strain 32
  source 1..1491: mol_type = genomic DNA
agagtttgatcctggctcaggatgaacgctggcggcgtgcttaacacatgcaagtcgaacgagaatcagtggattgaggattcgtccaaatgaaactgaggaaagtggcggacgggtgagtaacgcgtgagcaatctgccttggagtggggaataacggctggaaacagccgctaataccgcatgatacagttgggaggcatctctctgactgtcaaagatttatcgctctgagatgagctcgcgtctgattagctagttggcggggtaacggcccaccaaggcgacgatcagtagccggactgagaggttggccggccacattgggactgagacacggcccagactcctacgggaggcagcagtggggaatattgggcaatgggcgcaagcctgacccagcaacgccgcgtgaaggaagaaggctttcgggttgtaaacttcttttctgggggacgaacaaatgacggtaccccaggaataagccacggctaactacgtgccagcagccgcggtaatacgtaggtggcaagcgttatccggatttattgggtgtaaagggcgtgtaggcgggaatgcaagtcagatgtgaaaactatgggctcaacccatagcctgcatttgaaactgtatttcttgagtgctggagaggcaatcggaattccgtgtgtagcggtgaaatgcgtagatatacggaggaacaccagtggcgaagcggattgctggacagtaactgacgctgaggcgcgaaagcgtggggagcaaacaggattagataccctggtagtccacgccgtaacgatggatactagtgtggggggactgaccccctccgtgccgcagctaacgcaataagtatccccacctggggagtacgatcgcaaggttgaaactcaaaggaattgacgggggcccgcacaagcggtggagtatgtggtttaattcgaagcaacgcgaagaaccttaccagggcttgacatcctgctaacgaaccagagatggattaggtgcccttcggggaaagcagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggtggggacgacgtcaaatcatcatgccccttacgtcctgggccacacacgtactacaatggcggttaacaaagagaggcaagaccgcgaggtggagcaaatctcaaaaagccgtcccagttcggatcgcaggctgcaacccgcctgcgtgaagttggaatcgctagtaatcgcggatcagcatgccgcggtgaatacgttcccgggccttgtacacaccgcccgtcacaccatgagagtcgggaacacccgaagtccgtagcctaaccgcaaggggggcgcggccgaaggtgggttcgataattggggtgaagtcgtaacaaggtagccgt

SEQ ID NO: 53 - DNA, 1495 bp, Blautia coccoides
  rRNA 1..1495: note = 16S rRNA coding gene sequence of Clostridium strain 33
  source 1..1495: mol_type = genomic DNA
agagtttgatcctggctcaggatgaacgctggcggcgtgcctaacacatgcaagtcgaacgggtgtacggggaggaaggcttcggccggaaaacctgtgcatgagtggcggacgggtgagtaacgcgtgggcaacctggcctgtacagggggataacacttagaaataggtgctaataccgcataacgggggaagccgcatggcttttccctgaaaactccggtggtacaggatgggcccgcgtctgattagccagttggcagggtaacggcctaccaaagcgacgatcagtagccggcctgagagggcggacggccacactgggactgagacacggcccagactcctacgggaggcagcagtgggggatattgcacaatggggggaaccctgatgcagcgacgccgcgtgggtgaagaagcgcctcggcgcgtaaagccctgtcagcagggaagaaaatgacggtacctgaagaagaagccccggctaactacgtgccagcagccgcggtaatacgtagggggcaagcgttatccggatttactgggtgtaaagggggcgcagacggcgatgcaagccaggagtgaaagcccggggcccaaccccgggactgctcttggaactgcgtggctggagtgcaggaggggcaggcggaattcctggtgtagcggtgaaatgcgtagatatcagaggaacaccggtggcgaaagcggcctgctggactgcaactgacgttgaggcccgaaagcggtgggagcaaacaggattagataccctggtagtccacgccgtaaacgatgattactaggtgtcggggagcagagactgcccggtgccgcagcccaacgcattaagtatccacctggggagtacgttcgcaagaatgaaactcaaaggaattgacggggacccgcacaagcggtggagcatgtggtttaattcgaagcaacgcgaagaaccttaccaggccttgacatccccctggatggcccgtaacggggccagccctttttgggcaggggagacaggtggtgcatggttgtcgtcagctcgtgtcgtgagatgttgggttaagtcccgcaacgagcgcaacccctgcccgcagtagccagcattttagatggggactctgcggggactgccggggacaacccggaggaaggcggggatgacgtcaaatcatcatgccccttatggcctgggctacacacgtgctacaatggcgccgacagagggaggagaagcggcgacgcggagcgaaccccaaaaacggcgtcccagttcggattgtagtctgcaacccgactacatgaagccggaatcgctagtaatcgcggatcagaatgccgcggtgaatacgttcccgggtcttgtacacaccgcccgtcacaccatgggagccgggaatgcccgaagtctgtgaccgaacccgtaaggggaggggcagccgaaggcaggcccggtgactggggtgaagtcgtaacaaggtagccgt

SEQ ID NO: 54 - DNA, 1493 bp, Clostridium leptum
  rRNA 1..1493: note = 16S rRNA coding gene sequence of Clostridium strain 34
  source 1..1493: mol_type = genomic DNA
agagtttgatcctggctcaggacgaacgctggcggcgtgcttaacacatgcaagtcgaacggagcacccctgaaggagttttcggacaacggatgggaatgcttagtgtcggactggtgagtaacgcgtgaggaacctgccttccagagggggacaacagttggaaacgactgctaataccgcatgatgcgttggagccgcatgactccgacgtcaaagatttatcgctggaagatggcctcgcgtctgatttgttagttggtgaggtaacggcccaccaaggcgacgatcagtagccggactgagaggttggccggccacattgggactgagatacggcccagactcctacgggaggcagcagtggggaatattgggcaatggacgcaagtctgacccagcaacgccgcgtgaaggaagaaggctttcgggttgtaaacttcttttaagggggaagagcagaagacggtaccccttgaataagccacggctaactacgtgccagcagccgcggtaatacgtaggtggcaagcgttgtccggatttactgggtgtaaagggcgtgcagccggagagacaagtcagatgtgaaatccacgggctcaacccgtgaactgcatttgaaactgtttcccttgagtgtcggagaggtaatcggaattccttgtgtagcggtgaaatgcgtagatataaggaagaacaccagtggcgaaggcggattactggacgataactgacggtgaggcgcgaaagcgtggggagcaaacaggattagataccctggtagtccacgctgtaaacgatcgatactaggtgtgcggggactgaccccctgcgtgccggagttaacacaataagtatcgcacctggggagtacgatcgcaaggttgaaactcaaaggaattgacgggggcccgcacaagcggtggattatgtggtttaattcgaagcaacgcgaagaaccttaccagggcttgacatcctgctaacgaagtagagatacattaggtgcccttcggggaaagcagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggcggggacgacgtcaaatcatcatgccccttatgtcctgggctacacacgtaatacaatggcggttaacaaagggatgcaaagccgcgaggcagagcgaaccccaaaaagccgtcccagttcggatcgcaggctgcaacccgcctgcgtgaagtcggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgt

SEQ ID NO: 55 - DNA, 1498 bp, Blautia coccoides
  rRNA 1..1498: note = 16S rRNA coding gene sequence of Clostridium strain 35
  source 1..1498: mol_type = genomic DNA
agagtttgatcctggctcaggatgaacgctggcggcgtgcctaacacatgcaagtcgaacgggtgtacagaagggaagattacggtcggaaggtctgtgcatgagtggcggacgggtgagtaacgcgtgggcaacctggcctgtacagggggataacacttagaaataggtgctaataccgcataacgggggaagccgcatggcttttccctgaaaactccggtggtacaggatgggcccgcgtctgattattttttttgtcagggtaacggcctaccaaagcgacgatcagtagccggcctgagagggcggacggccacactgggactgagacacggcccagactcctacgggaggcagcagtgggggatattgcacaatggggggaaccctgatgcagcgacgccgcgtgggtgaagaagcgcctcggcgcgtaaagccctgtcagcagggaagaaaatgacggtacctgaagaagaagccccggctaactacgtgccagcagccgcggtaatacgtaaggggcaagcgttatccggatttactgggtgtaaagggggcgcagacggcgatgcaagccaggagtgaaagcccggggcccaaccccgggactgctcttgggaactgcggtggctggagtgcaggaggggcaggccggaattcctggtgtagcggtgaaatgcgtagatatcaggaggaacaccggtggcgaaggcggcctgctggactgcaactgacgttgaggcccgaaagcgtggggagcaaacaggattagataccctggtagtcacgccgtaaacgatgattactaggtgtcggggagcagagactgcccggtgccgcagccaacgcattaagtaatccacctggggagtacgttcgcaagaatgaaactcaaaggaattgacggggacccgcacaagcggtggagcatgtggtttaattcgaagcaacgcgaagaaccttaccaggccttgacatccccctggatggcccgtaacggggtcagcctttcggggcaggggagacaggtggtgcatggttgtcgtcagctcgtgtcgtgagatgttgggttaagtcccgcaacgagcgcaacccctgcccgcagtagccagcattttagatggggactctgcggggactgccggggacaacccggaggaaggcggggatgacgtcaaatcatcatgccccttatggcctgggctacacacgtgctacaatggcgccgacagagggaggcgaagcggcgacgcggagcgaaccccaaaaacggcgtcccagttcggattgtagtctgcaacccgactacatgaagccggaatcgctagtaatcgcggatcagaatgccgcggtgaatacgttcccgggtcttgtacacaccgcccgtcacaccatgggagccgggaatgcccgaagtctgtgaccgaacccgtaaggggaggggcagccgaaggcaggcccggtgactggggtgaagtcgtaacaaggtagccgt

SEQ ID NO: 56 - DNA, 1491 bp, Clostridium leptum
  rRNA 1..1491: note = 16S rRNA coding gene sequence of Clostridium strain 36
  source 1..1491: mol_type = genomic DNA
agagtttgatcatggctcaggacgaacgctggcggcaagcttaacacatgcaagtcgaacggagcgcctatgaaggagatttcggtcaacggaataggctgcttagtggctgacgggtgagtaacgcgtgaggaacctgcctttcagagggggacaacagttggaaacgactgctaataccgcataacacataggtgtcgcatggcatttatgtcaaagatttatcgctgaaagatggcctcgcgtctgattagctagttggtgaggtaacggctcaccaaggcgacgatcagtagccggactgagaggttagccggccacattgggactgagatacggcccagactcctacgggaggcagcagtggggaatattgggcaatggacgcaagtctgacccagcaacgccgcgtgaaggaagaaggctttcgggttgtaaacttcttttaagagggaagagcagaagacggtacctcttgaataagccacggctaactacgtgccagcagccgcggtaatacgtagtggcaagcgttgtccggatttactgggtgtaaagggcgtgtagccgggctgacagtcagatgtgaaattccggggctcaaccccggacctgcatttgaaactgttggtcttgagtatcggagaggcaggcggaattcctagtgtagcggtgaaatgcgtagatattaggaggaacaccagtggcgaaggcggcctgctggacgacaactgacggtgaggcgcgaaagcgtggggagcaaacaggattagataccctggtagtccacgctgtaaacgatggatactaggtgtgcggggactgaccccctgcgtgccgcagttaacacaataagtatcccacctggggagtacgatcgcaaggttgaaactcaaaggaattgacgggggcccgcacaagcggtggattatgtggtttaattcgatgcaacgcgaagaaccttaccagggcttgacatcctgctaacgaggtagagatacgtcaggtgcccttcggggaaagcagagacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccccttatgtcctgggctacacacgtaatacaatggcggtaaacagagggatgcaatactgcgaagtggagcgaacccctaaaagccgtcccagttcagattgcagtctgcaactcgactgcatgaagtcggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgt

SEQ ID NO: 57 - DNA, 1493 bp, Blautia coccoides
  rRNA 1..1493: note = 16S rRNA coding gene sequence of Clostridium strain 37
  source 1..1493: mol_type = genomic DNA
agagtttgatcctggctcaggatgaacgctggcggcgtgcctaacacatgcaagtcgaacgggtgtacggggaggaaggcttcggccggaaaacctgtgcatgagtggcggacgggtgagtaacgcgtgggcaacctggcctgtacagggggataacacttagaaataggtgctaataccgcataacgggggaagccgcatggcttttccctgaaaactccggtggtacaggatgggcccgcgtctgattagccagttggcagggtaacggcctaccaaagcgacgatcagtagccggcctgagagggcggacggccacactgggactgagacacggcccagactcctacgggaggcagcagtgggggatattgcacaatggggggaaaccctgatgcagcgacgccgcgtgagtgaagaagtatttcggtatgtaaagctctatcagcagggaagaaaatgacggtacctgactaagaagccccggctaactacgtgccagcagccgcggtaatacgtagggggcaagcgttatccggatttactgggtgtaaagggagcgtagacggcagcgcaagtctgaagtgaaatcccatggcttaaccatggaactgctttggaaactgtgcagctggagtgcaggagaggtaagcggaattcctagtgtagcggtgaaatgcgtagatattaggaggaacaccagtggcgaaggcggcttactggactgtactgacgttgaggctcgaaagcgtggggagcaaacaggattagataccctggtagtccacgccgtaaacgatgattactaggtgttgggggaccaaggtcttcggtgccggcgcaaacgcattaagtaatccacctggggagtacgttcgcaagaatgaaactcaaaggaattgacggggacccgcacaagcggtggagcatgtggtttaattcgaagcaacgcgaagaaccttacctggtcttgacatcccgatgacgagtgagcaaagtcactttcccttcggggcattggagacaggtggtgcatggttgtcgtcagctcgtgtcgtgagatgttgggttaagtcccgcaacgagcgcaacccctatttccagtagccagcaggtagagctgggcactctggagagactgcccgggataaccgggaggaaggcggggatgacgtcaaatcatcatgccccttatgatcagggctacacacgtgctacaatggcgtaaacaaagggaagcgagacggtgacgttgagcaaatcccaaaaataacgtcccagttcggattgtagtctgcaactcgactacatgaagctggaatcgctagtaatcgcgaatcagaatgtcgcggtgaatacgttcccgggtcttgtacacaccgcccgtcacaccatgggagtcggaaatgcccgaagtcagtgacctaaccgaaaggaaggagctgccgaaggtggagccggtaactggggtgaagtcgtaacaaggtagccgt

SEQ ID NO: 58 - DNA, 1493 bp, Clostridium leptum
  rRNA 1..1493: note = 16S rRNA coding gene sequence of Clostridium strain 38
  source 1..1493: mol_type = genomic DNA
aaagtttgatcctggctcaggacgaacgctggcggcgtgcttaacacatgcaagtcgaacggagcacccctgaaggagttttcggacaacggatgggaatgcttagtggcggactggtgagtaacgcgtgaggaacctgccttccagagggggacaacagttggaaacgactgctaataccgcatgatgcgttggagccgcatgactccgacgtcaaagatttatcgctggaagatggcctcgcgtctgattagctagttggtgaggtaacggcccaccaaggcgacgatcagtagccggactgagaggttggccggccacattgggactgagatacggcccagactcctacgggaggcagcagtggggaatattgggcaatggacgcaagtctgacccagcaacgccgcgtgaaggaagaaggctttcgggttgtaaacttcttttaagggggaagagcagaagacggtaccccttgaataagccacggctaactacgtgccagcagccgcggtaatacgtagtggcaagcgttgtccggatttactgggtgtaaagggcgtgcagccggagagacaagtcagatgtgaaatccacgggctcaacccgtgaactgcatttgaaactgtttcccttgagtgtcggagaggtaatcggaattccttgtgtagcggtgaaatgcgtagatataaggaagaacaccagtggcgaaggcggattactggacgataaactgacggtgaggcgcgaaagcgtggggagcaaacaggattagataccctggtagtccacgctgtaaacgatcgatactaggtgtgcggggactgaccccctgcgtgccggagttaacacaataagtatcgcacctggggagtacgatcgcaaggttgaaactcaaaggaattgacgggggcccgcacaagcggtggattatgtggtttaattcgaagcaacgcgaagaaccttaccagggcttgacatcctgctaacgaagtagagatacattaggtgcccttcggggaaagtagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggcggggacgacgtcaaatcatcatgccccttatgtcctgggctacacacgtaatacaatggcggttaacaaagggatgcaaagccgcgaggcagagcgaaccccaaaaagccgtcccagttcggatcgcaggctgcaacccgcctgcgtgaagtcggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgt

SEQ ID NO: 59 - DNA, 1511 bp, Clostridium leptum
  rRNA 1..1511: note = 16S rRNA coding gene sequence of Clostridium strain 39
  source 1..1511: mol_type = genomic DNA
agagtttgatcctggctcaggacgaacgctggcggcgtgcttaacacatgcaagtcgaacggagcacccctgaaggagttttcggacaacggatgggaatgcttagtggcggactggtgagtaacgcgtgaggaacctgccttccagagggggacaacagttggaaacgactgctaataccgcatgatgcgttggagccgcatgactccgacgtcaaagatttatcgctggaagatggcctcgcgtctgattagctagttggtgaggtaacggcccaccaaggcgacgatcagtagccggactgagaggttggccggccacattgggactgagatacggcccagactcctacgggaggcagcagtggggaatattgggcaatggacgcaagtctgacccagcaacgccgcgtgaaggaagaaggctttcgggttgtaaacttcttttaagggggaagagcagaagacggtaccccttgaataagccacggctaactacgtgccagcagccgcggtaatacgtaggtggcaagcgttgtccggatttactgggtgtaaagggcgtgcagccggagagacaagtcagatgtgaaatccacgggctcaacccgtgaactgcatttgaaactgtttcccttgagtgtcggagaggtaatcggaattccttgtgtagcggtgaaatgcgtagatataaggaagacaccagtggcgaagcggattactggacgataactgacggtgaggcgcgaaagcgtggggagcaaacaggattagatacctgggtagtcaacgctgtaaacgatcgatactaggtggtgcgggggacttgaccccctgccgttgccggagttaacaccaataaagtattcggcaccctggggagtacgatcgcaaaggttgaaaactcaaaagaaatggacggggggccccgccccaagcgggtgggattatgttggtttatttcgaaagcaacgcgaagaaccctaacagggcttgacatcctgctaacgaagtagagatacattaggtgcccttcggggaaagtagagacaggtggtgcatggttgtcgtcagctcgtgtcgtgagatgttgggttaagtcccgcaacgagcgcaacccctattgttagttgctacgcaagagcactctagcgagactgccgttgacaaaacggaggaaggcggggacgacgtcaaatcatcatgccccttatgtcctgggctacacacgtaatacaatggcggttaacaaagggatgcaaagccgcgaggcagagcgaaccccaaaaagccgtcccagttcggatcgcaggctgcaacccgcctgcgtgaagtcggaatcgctagtaatcgcggatcagcatgccgcggtgaatacgttcccgggccttgtacacaccgcccgtcacaccatgagagtcgggaacacccgaagtccgtagcctaaccgcaaggagggcgcggccgaaggtgggttcgataattggggtgaagtcgtaacaaggtagccgt

SEQ ID NO: 60 - DNA, 1499 bp, Clostridium leptum
  rRNA 1..1499: note = 16S rRNA coding gene sequence of Clostridium strain 40
  source 1..1499: mol_type = genomic DNA
agagtttgatcctggctcaggataaacgctggcggcatgcctaacacatgcaagtcgaacggagcgccttggaaggagacttcggtcaacggaagaggaggcttagtggcggacgggtgagtaacgcgtgaggaacctgcctcagagagggggataacacaccgaaaggtgtgctaataccgcataacatatgagaggggcatccctttcatatcaaagatttattgctttgagatggcctcgcgtccaattagctagttggtgaggtaacggcccaccaaggcgacgattggtagccggactgagaggttgaacggccacattgggactgagacacggcccagactcctacgggaggcagcagtggggaatattgcacaatggggggaaccctgatgcagcaatgccgcgtgaaggatgaaggttttcggattgtaaacttcttttgtacgggacgaagaaagtgacggtaccgtaagaataagccacggctaactacgtgccagcagccgcggtaatacgtaggtggcaagcgttatccggatttactgggtgtaaagggcgagtaggcgggattgcaagtcagatgtgaaaactatgggctcaaccgatagagtgcatttgaaactgcagttcttgagtgatggagaggcaggcggaattcccggtgtagcggtggaatgcgtagatatcgggagggaacaccagtggcgaaggcggcctgctggacattaactgacgctgatgcgcgaaagcgtggggagcaaacaggattagataccctggtagtcacgctgtaaacgatgattactaggtgtggggggtactgacccccttcccgtgccggagttaacacaataagtaatccacctggggagtacggccgcaaggttgaaactcaaaggaattgacgggggcccgcacaagcagtggagtatgtggttttaattcgaagcaacgcgaagaaccttaccagggcttgacatggggatgaccgctttagagatagagctttctcttcggagacatcccacacaggtggtgcatggttgtcgtcagctcgtgtcgtgagatgttgggttaagtcccgcaacgagcgcaacccttattgttagttgctacgcaagagcactctagcgagactgccgttgacaaaacggaggaaggtggggacgacgtcaaatcatcatgccctttatgtcctgggctacacacgtactacaatggcggacatacagagggaagcaagacagcgatgtggagcaaatccctaaaagccgtctcagttcagattgcaggctgcaacccgcctgcatgaagtcggaattgctagtaatcgcggatcagcatgccgcggtgaatacgttcccgggccttgtacacaccgcccgtcacaccatgagagtcggaaacacccgaagcctgtagcccaaccgcaaggggggcgcagtcgaaggtgggtctgataattggggtgaagtcgtaacaaaggtagccgt

SEQ ID NO: 61 - DNA, 1512 bp, Blautia coccoides
  rRNA 1..1512: note = 16S rRNA coding gene sequence of Clostridium strain 41
  source 1..1512: mol_type = genomic DNA
agagtttgatcctggctcaggatgaacgctggcggcgtgcttaacacatgcaagtcgaacggagatatcattttcgaagcgattagtttactaagagcggagatgttgctatcttagtggcggacgggtgagtaacgcgtgggtaacctgccttgcactgggggataacacttagaaataggtgctaataccgcataacagtaggagacgcatgtcttttacttgaaaactccggtggtgtaagatggacccgcgtctgattagcttgttggcggggtaacggcccaccaaggcaacgatcagtagccggcctgagagggtgaacggccacattgggactgagacacggcccaaactcctacgggaggcagcagtggggaatattggacaatggggggaaccctgatccagcgacgccgcgtgagtgaagaagtatttcggtatgtaaagctctatcagcagggaagaaagaaatgacggtacctgactaagaagccccggctaactacgtgccagcagccgcggtaatacgtagggggcaagcgttatccggatttactgggtgtaaagggagcgtagacggcgatgcaagtctgaagtgaaaggcgggggcccaacccccggactgctttggaaactgtatggctggagtgcaggagaggtaagtggaattcctagtgtagcggtgaaatgcgtagatattaggaggaacaccagtggcgaaagcggcttactggactgtaactgacgttgaggctcgaaagcgtggggagcaaacaagattagatacctggtagtcacgccgtaaacgatgatcaccggtttcggtgggttatggacccatcggttgcgcagcaaacgcagtagtgatccacctggggagtaacgttcgcaagaatgaaacttcaaaggaaatgacgggggacccggcacaagcggtggaggcatgtgtttaattcgaagcaacgcgaagaaccttacccaagtcttgacatcccgtgacgagtgagtaacgtcactttcccttcggggcagcggagacaggtggtgcatggttgtcgtcagctcgtgtcgtgagatgttgggttaagtcccgcaacgagcgcaacccctatccttagtagccagcgagttaggtcgggcactctagggagactgccggggacaacccggaggaaggtggggatgacgtcaaatcatcatgccccttatgatttgggctacacacgtgctacaatggcgtaaacaaagggaagcgagcctgtgaaggtaagcgaatcccagaaataacgtctcagttcggattgtagtctgcaactcgactacatgaagctggaatcgctagtaatcgcggatcagaatgccgcggtgaatacgttcccgggtcttgtacacaccgcccgtcacaccatgggagtcggaaatgcccgaagtctgtgacccaacctgagaaggagggagcagccgaaggcaggtcggatgactggggtgaagtcgtaacaaggtagccgt

SEQ ID NO: 62 - DNA, 17 bp, synthetic construct
  misc_feature 1..17: note = Artificially synthesized primer sequence
  source 1..17: mol_type = other DNA
ggtgaatacgttcccgg

SEQ ID NO: 63 - DNA, 22 bp, synthetic construct
  misc_feature 1..22: note = Artificially synthesized primer sequence
  source 1..22: mol_type = other DNA
tacggctaccttgttacgactt

SEQ ID NO: 64 - DNA, 20 bp, synthetic construct
  misc_feature 1..20: note = Artificially synthesized primer sequence
  source 1..20: mol_type = other DNA
aaatgacggtacctgactaa

SEQ ID NO: 65 - DNA, 22 bp, synthetic construct
  misc_feature 1..22: note = Artificially synthesized primer sequence
  source 1..22: mol_type = other DNA
ctttgagtttcattcttgcgaa

SEQ ID NO: 66 - DNA, 16 bp, synthetic construct
  misc_feature 1..16: note = Artificially synthesized primer sequence
  source 1..16: mol_type = other DNA
gcacaagcagtggagt

SEQ ID NO: 67 - DNA, 18 bp, synthetic construct
  misc_feature 1..18: note = Artificially synthesized primer sequence
  source 1..18: mol_type = other DNA
cttcctccgttttgtcaa

SEQ ID NO: 68 - DNA, 18 bp, synthetic construct
  misc_feature 1..18: note = Artificially synthesized primer sequence
  source 1..18: mol_type = other DNA
gagaggaaggtcccccac

SEQ ID NO: 69 - DNA, 19 bp, synthetic construct
  misc_feature 1..19: note = Artificially synthesized primer sequence
  source 1..19: mol_type = other DNA
cgctacttggctggttcag